(19) United States
(12) Patent Application Publication
(10) Pub. No.: US 2007/0166816 A1
(43) Pub. Date: Jul. 19, 2007

(54) Assay device that analyzes the absorption, metabolism, permeability and/or toxicity of a candidate compound

(75) Inventors: Stewart Campbell, Framingham, MA (US); Enoch Kim, Boston, MA (US); Gregory L. Kirk, Winchester, MA (US); Emanuele Ostuni, Watertown, MA (US); Olivier Schueller, Arlington, MA (US); Rocco Casagrande, Newton, MA (US); Evelyn Wang, Haddonfield, NJ (US); Paul Sweetnam, Marblehead, MA (US)

Correspondence Address: KENYON & KENYON LLP, 1500 K STREET N.W., SUITE 700, WASHINGTON, DC, 20005, US

(73) Assignee: Surface Logix, Inc., Brighton, MA (US)

(21) Appl. No.: 11/637,912

(22) Filed: Dec. 13, 2006

Related U.S. Application Data
(62) Division of application No. 10/387,949, filed on Mar. 12, 2003, now abandoned.
(60) Provisional application No. 60/363,735, filed on Mar. 12, 2002.
(60) Provisional application No. 60/374,800, filed on Apr. 24, 2002.

(51) Int. Cl. C12M 1/34

(52) U.S. Cl. 435/288.4; 435/288.5

(57) ABSTRACT

This invention provides device for co-culturing at least two different cell types in a two-dimensional configuration, methods of patterning at least two different cell types in a two-dimensional co-culture configuration, and uses of these devices and methods for analyzing an effect of candidate compound on such cellular cocultures. Also provided is a transmigration and extravasation device. Assay devices for analyzing the absorption, permeability, metabolism and/or toxicity of a candidate compound by a cell are provided. A microfluidic network, which is adaptable for integration with a device for coculturing is provided.

This application claims the benefit of U.S. Provisional Application No. 60/363,735, filed Mar. 12, 2002 and U.S. Provisional Application No. 60/374,800, filed Apr. 24, 2002. The entire disclosures of the prior applications are hereby expressly incorporated by reference in their entirety.

### FIELD OF THE INVENTION

The invention relates to methods and devices for analyzing the adsorptive, metabolic and toxic characteristics of experimental compounds drugs, drug candidates, food-shifts and toxins on cells. The present invention generally relates to high-throughput, flexibly formatted cell based assays. The devices used in these cell-based assays include multi-well platforms that can be used in automated and integrated systems, and include methods for rapidly identifying chemicals having biological activity in liquid samples, and in particular use automated screening of low volume samples to identify new medicines, agrochemicals, or cosmetics.

### BACKGROUND OF THE INVENTION

In order to find candidate drug compounds that either inhibit or stimulate a particular enzyme cascade, it is necessary to develop multi-well high-throughput assays. Once identified, candidate drugs or modulators are usually evaluated for bioavailability and toxicological effects. See Lu, Basic Toxicology, Fundamentals, Target Organs, and Risk Assessment, Hemisphere Publishing Corp., Washington (1985); U.S. Pat. No. 5,196,313 to Culbreth (issued Mar. 23, 1993) and U.S. Pat. No. 5,567,592 to Benet (issued Oct. 22, 1996). Traditionally, early stages of drug discovery and development have concentrated on optimizing binding and potency of experimental compounds. Typically, animal studies are performed on late stage pre-clinical drug candidates to characterize pharmacokinetics (PK), pharmacodynamics (PD) and physiological toxicity. However, animal studies are costly, time-consuming and are limited, by throughput, to characterize no more than a few compounds. Furthermore, several drugs have shown unanticipated or unpredicted side effects only after reaching clinical trials or wide-scale release to the public. The pharmaceutical industry has the ultimate goal of replacing animal studies with in vitro tests that are validated, predictive models for human toxicity and drug dynamics. More recently, the industry has set a medium-term goal of creating high-throughput, in vitro tests that annotate candidate compounds with adsorption, metabolism and toxic (hereinafter referred to as “ADMET”) predictive parameters.

The toxicology of a candidate modulator can be established by determining in vitro toxicity towards a cell line, such as a mammalian, including human, cell lines. Candidate modulators can be treated with, for example, tissue extracts, such as preparations of liver (such as microsomal preparations) to determine increased or decreased toxicological properties of the chemical after being metabolized by a whole organism. The results of these types of studies are often predictive of toxicological properties of chemicals in animals, such as mammals, including humans. Current methods designed to model drug absorption in vivo involve growing a confluent layer of cells on a porous matrix that allows the test compound to permeate through the cell layer and matrix to a bottom well. This system requires the formation of tight junctions between the cells of the monolayer so that no test compound permeates the monolayer without passing through a cell. In the past, these systems have suffered from “leaky” junctions that allow the test compound to by pass flowing through the cells and instead pass directly through the membrane. None of these techniques are amenable to high-through put screening as they are time consuming, expensive, require numerous amounts of cells and test solutions and, as discussed above, are prone to error. The need for information regarding drug candidate/cellular absorption and metabolism has created a need for sensitive assays enough to concurrently test, in a cost-effective manner, vast arrays of compounds for interactions with the cells involved in that particular drug's metabolism.

Developing an in vitro system that can accurately predict in vivo effects is technically challenging for several reasons. Primarily, the complexity and interplay of biological processes that must be simulated to predict the ADMET properties of a compound far exceed the capabilities of currently available tools and methods. For example, when a patient takes a drug, it must first pass through the gastrointestinal tract and penetrate into the bloodstream. The drug must then survive oxidative modifications in the liver and get to the desired site (e.g., target organ or primary tumor) in a sufficient therapeutic concentration. Even if these biological functions could be faithfully reproduced in vitro, a difficulty remains in getting the capacity and format of the assay to facilitate testing and analysis of thousands of compounds.

Another challenge is functional miniaturization: the integration of micro-assay elements in a format compatible with standard fluid handling tools and plate handling robots. Therefore, these multi-well high-throughput assays must be able to monitor activation or inhibition of the enzyme cascade inside living or whole cells. Ideally, the assays should be versatile enough to not only measure the enzyme cascade activity inside any living or whole cell, no matter what its origin might be, including cancer cells, tumor cells, immune cells, brain cells, cells of the endocrine system, cells or cell lines from different organ systems, biopsy samples etc., but should also be able to detect and measure the permeability of the cell to the candidate compound, as well as the metabolic activity of the cell on the candidate drug compound. Developing such versatile assays would represent a substantial advance in the field of drug screening. Methodologies are therefore desired that will allow for the more rapid acquisition of information about drug candidate interactions with enzymes that may potentially metabolize the candidate drug, earlier in the drug discovery process than presently feasible. This will thus allow for the earlier elimination of unsuitable compounds and chemical series from further development efforts, and also give the investigator insight as to the nature of metabolites with potential biological activity derived from the candidate drug.

By way of example, liver hepatocytes express a family of enzymes called cytochromes. One subfamily of cytochromes is known as cytochrome P450. The cytochrome P450 enzyme (CYP450) family comprises oxidase enzymes involved in the xenobiotic metabolism of hydrophobic drugs, carcinogens, and other potentially toxic compounds and metabolites circulating in blood. Efficient metabolism of a candidate drug by a CYP450 enzyme may lead to poor pharmacokinetic properties, while drug candidates that act as potent inhibitors of a CYP450 enzyme can cause undesirable drug-drug interactions when administered with another drug that interacts with the same CYP450. See, e.g., Peck, C. C. et al, Understanding Consequences of Concurrent Therapies, 269 JAMA 1550-52 (1993). Accordingly, early, reliable indication that a candidate drug interacts with (i.e., is absorbed by, metabolized by, or toxic to) hepatocytes expressing CYP450 may greatly shorten the discovery cycle of pharmaceutical research and development, and thus may reduce the time required to market a candidate drug. Consequently, such earlier-available, reliable pharmacokinetic information may result in greatly reduced drug development costs and/or increased profits from earlier market entrance. Furthermore, such earlier-available, reliable pharmacokinetic information may allow a candidate drug to reach the public sooner, at lower costs than otherwise feasible. Accordingly, extensive pharmacokinetic studies of drug interactions in humans have recently become an integral part of the pharmaceutical drug development and safety assessment process. See, e.g., Parkinson, A., 24 Toxicological Pathology 45-57 (1996).

Previous methods and assay devices have used layers of cells or monolayers of cells to test the effects of compounds on such cells for permeability, biological activity, toxicity and teratogenicity. For example, U.S. Pat. No. 5,602,028 to Minchinton issued Feb. 11, 1997 describes an assay device consisting of a plurality of layers including a chamber for submerging cells in a medium while supporting and binding the cells to a semi-permeable membrane, wherein such device may be used for measuring the rate of penetration by an agent through a cell culture.

Gabriels, Jr., U.S. Pat. No. 5,175,092, issued Dec. 29, 1992 describes a method for producing cells (e.g., keratinocytes, epithelial or endothelial cells) in vitro, which are grown in a monolayer or a differentiated tissue on a collagen coated polymeric microporous cell growth substrate coated with cell growth supporting material, which may be used to determine the toxic effects of a substance on the tissue by exposing the tissue to the substance and evaluating cellular response to the substance.

U.S. Pat. No. 6,046,056 to Parce et al. issued Apr. 4, 2000 describes microfluidic devices for performing high-throughput screening assay for an effect of a test compound on a flowing biochemical system.

PCT International Application No. WO 97/16717 of Eli Lilly and Co, published May 9, 1997 describes an automated permeability analysis device.

U.S. Pat. No. 6,103,199 to Bjornson et al. issued Aug. 15, 2000 describes an apparatus for conducting a microfluidic process.

PCT International Application No. WO 99/47922 to Massachusetts Institute of Technology, published Sep. 23, 1999 and U.S. Pat. No. 6,197,575 to Griffith et al. issued Mar. 6, 2001, describe an apparatus comprising a matrix which includes one or more channels to support the viability of cells, cells within the channels of the matrix, and means for detecting changes in the cells or in compounds exposed to the cells.

PCT International Application No. WO 99/28437 to Tam et al. published Jun. 10, 1999 and U.S. Pat. No. 6,022,733 to Tam et al. issued Feb. 8, 2000, describe a system and a method for assessing simulated biological dissolution of a pharmaceutical formulation and absorption of a pharmaceutically active compound therefrom.

U.S. Patent Publication No. US 2002/001541 of Holden et al., published Jan. 3, 2002, describes a device to evaluate the quality of a combinatorial library of compounds.

U.S. Pat. No. 5,160,490 to Naughton et al. issued Nov. 3, 1992 describes a three-dimensional cell culture system to culture cells and tissues in vitro, wherein parenchymal cells are inoculated and grown on living stromal matrix, the stromal matrix comprising stromal cells growing on a three-dimensional matrix.

Conventional co-culture techniques for combinations of cells have seeded one cell type on a substrate and seeded a second cell type on top of the first cell type. Kuri-Harcuch et al., U.S. Pat. No. 4,914,032, issued Apr. 3, 1990 describes a process for the long-term surviving culture of hepatocytes cocultured with multiplication inhibited fibroblasts or fibroblast products. Kuri-Harcuch et al., U.S. Pat. No. 5,030,105, issued Jul. 9, 1991 further describes a method of assessing test agents by treating the above-described long-term surviving cultured hepatocytes cocultured with fibroblasts or fibroblast products with test agents and assessing said hepatocytes for injury.

U.S. Pat. No. 5,518,915 to Naughton et al. issued May 21, 1996 describes a three-dimensional mucosal cell culture comprising mucosal epithelial cells cultured on living stromal tissue prepared in vitro, attached to and enveloping a framework composed of a biocompatible, non-living material formed into a three-dimensional structure having interstitial spaces bridges by the stromal cells.

Dunn et al., U.S. Pat. No. 5,602,026, issued Feb. 11, 1997, describes a method for maintaining hepatocytes in culture comprising contacting the hepatocytes with a support comprising two layers; the hepatocytes may be sandwiched between two supports, one of which comprises sterilized collagen. The hepatocytes may also be immobilized in collagen beads. Such a compositions of hepatocytes may be used as an extracorporeal perfusion system, as described by Dunn et al., U.S. Pat. No. 5,942,436, issued Aug. 24, 1999.

Automated in vitro cell culture systems, such as that described by Shuler et al. U.S. Pat. No. 5,612,188, issued Mar. 18, 1997, may be used to evaluate cells contacted with culture medium to which a substance to be evaluated has been added for physiological and metabolic changes resulting from the presence of the substance. As discussed below, the cells to be evaluated may be transfected with a human gene.

Specific cell cultures have also been used to assess bioavailability and potential drug-drug interactions of pharmacological agents. For example, Watkins et al., U.S. Pat. No. 5,856,189, issued Jan. 15, 1999 describes a cell line (Caco-2 cells) with enhanced expression of a member of the CYP3A subfamily which is made by plating the cells on an extracellular matrix, treating the plated cells with an analog of vitamin D₃, and isolating the treated cells that exhibit enhanced expression of the CYP3A subfamily. Such cells may be seeded onto a support matrix for use in a system to screen for the bioavailability of compounds.

Cells may also be modified by genetic engineering or transduction to express genetic material of interest. For example, Mulligan et al., U.S. Pat. No. 5,521,076, issued May 28, 1996, describes transduced mammalian hepatocytes having genetic material stably incorporated therein and capable of expression of the protein or polypeptide encoded by the genetic material, which comprises a retroviral vector lacking a selectable marker, e.g., α-SGC and MFG vectors.

Reporter genes may be introduced into cells so that such candidate drugs may be assessed by treating these cells with the drugs. For example, Singer et al., U.S. Pat. No. 5,556,754, issued Sep. 17, 1996, describes methods for assessing the therapeutic potential of a candidate drug for treating autoimmune diseases or transplantation rejection by assessing the ability of the drug to suppress MHC Class I molecules using cells having a reporter gene operably linked to a MHC Class I regulatory sequence.

Virally-immortalized mammalian cells, e.g., hepatocytes, may be used to evaluate the toxicity of a compound in vitro, as described by Jauregui et al, U.S. Pat. No. 5,869,243, issued Feb. 9, 1999 and Jauregui et al, U.S. Pat. No. 6,107,043, issued Aug. 22, 2000, by contacting such hepatocytes with a compound and measuring the viability of the hepatocyte.

Recombinantly modified cells may be used in assays for generating and analyzing stimulus-response output, e.g., transcriptional responsiveness of a living cell to a drug candidate, as described by Rine et al., U.S. Pat. No. 6,326,140 B1, issued Dec. 4, 2001.

Harris et al., U.S. Pat. No. 5,660,986, issued Aug. 26, 1997 describes non-tumorigenic stable, human bronchial and liver epithelial cell lines capable of expressing exogenous human cytochrome P450 genes which have been inserted into said cell lines, which may be used in methods of identifying or testing agents for mutagenicity, cytotoxicity or carcinogenicity by culturing said cells with a test agent and determining its effect on the cell line.

Human pluripotent stem cells have been directly differentiated without formation of embryoid bodies, in a monolayer culture on a suitable solid surface for use in drug screening, as described by U.S. Patent Application Publication No. US 2002/0019046 A1, published Feb. 14, 2002.

More recently, micropatterning techniques have been used to co-cultivate cells. Bhatia et al., PCT International Application No. WO 98/51785, published Nov. 19, 1998 and U.S. Pat. No. 6,133,030, issued Oct. 17, 2000, describes methods for producing co-cultures of at least two cell types in a micropattern configuration, each of which is expressly incorporated herein by reference in its entirety, specifically techniques and materials for coculturing of at least two cell types in a micropattern configuration, and methods of photolithographic patterning to produce a micropattern for such coculture. Briefly, a micropatterned co-culture is produced providing a substrate coated with a cell-binding protein which defines a micropattern on the substrate; contacting the cell-binding protein with cells of a first cell type suspended in a first cell medium under conditions such that the cells bind to the cell-binding protein, thereby producing a micropatterned cell-coated substrate; contacting the micropatterned cell-coated substrate with cells of a second cell type suspended in a second cell medium under conditions such that the cells of the second type bind to the substrate, thereby producing the micropatterned co-culture; one of the cell media is a selective medium that lacks serum and attachment factors and/or includes a non-adhesive factor to inhibit attachment and one of the cell media is an attachment medium that contains an effective amount of serum and/or at least one attachment factor. Such co-cultures may be used to modulate (e.g., upregulate or downregulate) a metabolic or synthetic function of either the first or second cell type, as described in Bhatia et al., U.S. Pat. No. 6,221,663 B1, issued Apr. 24, 2001, which is expressly incorporated herein by reference in its entirety, specifically techniques for modulating the metabolic or synthetic activity of cells cocultured in a micropattern configuration.

### SUMMARY OF THE INVENTION

The current invention is based on the permeability across a monolayer of cells as in conventional hepatocyte or Caco2 or MDCK absorption systems, but with major differences: 1) the present invention does not rely on the formation of tight junctions between cells, which have contributed to the difficulty and reproducibility of conventional absorption tests; 2) test compound absorption, permeability, metabolism and toxicity is determined in flow conditions at physiologically relevant levels; and 3) the format of the test assays and devices is amenable to high-throughput screening formats.

The metabolism/toxicology system of the presentation is based on the observation that hepatocytes in appropriate heterotypic culture retain their phenotypes and functionality for several months. The invention utilizes an in vitro metabolism system using co-culture of hepatocytes and supporting cells types, such as fibroblasts. Further, the present invention contemplates the use of a cell library representing the major human cell types for use in a high-throughput toxicology assay. The cells in this library may be transfected in situ to introduce a reporter system for rapid read out of toxicity.

The absorption and metabolism/toxicology systems, are integrated using appropriate microfluidics and detection schemes. This integrated ADMET system is designed to accurately model in vivo absorption, oxidative metabolic process in the liver, and toxicological effects on multiple cell types. Furthermore, this system can characterize the effects of unknown chemical and toxin agents on the body and evaluate the dangers of prolonged low-level exposure to compounds encountered on the battlefield.

The present invention provides for an assay device that analyzes the absorption, permeability and/or metabolism of a candidate compound by a cell, said device having one or more test chambers which comprises a test compound delivery device, one or more patterning membranes having one or more test cells immobilized therein, and an analyte removal device.

In another aspect of the invention, the assay device has one test chamber which comprises a test compound delivery device, one or more patterning membranes having one or more test cells immobilized therein, and an analyte removal device.

In another aspect of the invention, the assay device has a plurality of test chambers which comprises a test compound delivery device, one or more patterning membranes having one or more test cells immobilized therein, and an analyte removal device, which are arraigned such that each test chamber sits in the well of standard 96-, 384-, or 1536-well microtiter plate.

The present invention provides for an assay device that analyzes the absorption, permeability and/or metabolism of a candidate compound by a cell, having one or more test chambers which comprise a test compound delivery device, a first patterning membrane having one or more Caco-2 test cells immobilized therein, a second patterning membrane downstream of the first patterning membrane, having one or more hepatocyte test cells immobilized therein and an analyte removal device.

The present invention provides for an assay device that analyzes the absorption, permeability and/or metabolism of a candidate compound by a cell, having one or more test chambers which comprise a test compound delivery device, a first patterning membrane having one or more hepatocyte test cells immobilized therein, a second patterning membrane downstream of the first patterning membrane, having one or more test compound target tissue test cells immobilized therein and an analyte removal device.

The present invention provides for an assay device that analyzes the absorption, permeability and/or metabolism of a candidate compound by a cell, having one or more test chambers which comprise a test compound delivery device, a first patterning membrane having one or more Caco-2 test cells immobilized therein, a second patterning membrane downstream of the first patterning membrane, having one or more hepatocyte test cells immobilized therein; a third patterning membrane downstream of the second patterning membrane, having one or more test compound target tissue test cells immobilized therein and an analyte removal device.

In another aspect of the invention the test chamber further comprises a filter membrane is positioned downstream of the patterning membrane.

In additional embodiments the assay devices are hepatocyte based assays, which are well suited for the identification of engineered biological agents and emerging pathogens that target the liver. These assay devices, or biosensors, provide a complete in vitro system that predicts the reaction of humans to environmental factors.

The present invention further provides a unique integrated assay device that allows for differentiation of an agent's mechanism of absorption as well as its effects on hepatotoxicity and metabolism.

In one example embodiment, this invention provides a device for co-culturing at least two different cell types in a two-dimensional configuration comprising a cell culture support surface; and a microfluidic system having a removable patterning membrane disposed on the cell culture support surface and a plurality of channels for flowing cells to surfaces exposed within the channels, wherein the channels are in conformal contact with the cell culture support surface and are parallel relative to each other and spaced apart relative to each other.

In one example embodiment, this invention provides a device for co-culturing at least two different cell types in a two-dimensional configuration comprising a cell culture support; and at least one removable membrane disposed on the cell culture support, wherein the membrane forms a stencil pattern on the cell culture support.

In another example embodiment, this invention provides a method of patterning at least two different cell types in a two-dimensional co-culture configuration comprising: a) providing a device having a cell culture support surface; and a microfluidic system having a removable patterning membrane disposed on the cell culture support surface and a plurality of channels for flowing cells to surfaces exposed within the channels, wherein the channels are in conformal contact with the cell culture support surface and are parallel relative to each other and spaced apart relative to each other; b) flowing cells of one tissue type through one set of alternating channels to form multiple rows of contiguous cells of a first tissue type within the channels, wherein the rows are parallel relative to each other and spaced apart relative to each other; c) removing the removable microfluidic patterning membrane from the cell culture support to form alternating rows of bare cell culture support contiguous with and parallel relative to the rows of contiguous cells of step (b); and d) flowing cells of a second tissue type through a second set of alternating channels to the alternating rows of bare cell culture support of step (c), to form rows of contiguous cells of the second tissue type contiguous with the rows of contiguous cells of the first tissue type on the cell culture support.

In another example embodiment, this invention provides a method of patterning at least two different cell types in a two-dimensional co-culture configuration comprising: a) providing a device having a cell culture support; and at least one removable membrane disposed on the cell culture support, wherein the membrane forms a stencil pattern on the cell culture support; b) applying cells of one tissue type to open areas formed by the stencil pattern, wherein the open areas are spaced apart relative to each other; c) removing the at least one removable membrane from the cell culture support to form bare areas of cell culture support; and d) applying cells of a second tissue type to the bare areas cell culture support.

In a further example embodiment, this invention provides a method of patterning at least two different cell types in a two-dimensional co-culture configuration comprising: a) providing a non-coated cell growth substrate, wherein the substrate has a plurality of patterned electrodes embedded within said substrate and a plurality of electroactive cytophobic self-assembled monolayers (SAMs) patterned onto the cell substrate; b) applying cells of a first tissue type to the non-SAM coated cell growth substrate; c) desorbing the plurality of electroactive cytophobic SAMs from the cell substrate to form cell adhesive regions in the pattern of the removed SAMs; d) activating at least one electrode to form at least one activated region of the cell growth substrate; e) applying cells of a second cell type to the at least one activated region of step (d) to form a pattern the cells of the second cell type in at least one activated region, thereby patterning at least two different cell types in a two-dimensional co-culture configuration.

In one example embodiment, this invention provides a device comprising: at least three layers, said layers being a first layer, a top layer and a middle layer, wherein the first layer is a lower layer having fluid inlet receptacles and fluid outlet receptacles, said receptacles being connected by a microfluidic system, wherein the top layer has a cell culture well and an opening to said fluid inlet receptacle and fluid outlet receptacles and wherein the middle layer is configured to receive cells on its top surface, said layer being porous and separating the cell culture well from the microfluidic system.

In another example embodiment, this invention provides a device comprising: a housing defining at least one chamber therein; a membrane disposed in the at least one chamber and defining a plurality of micro-orifices, the membrane being configured such that each of the plurality of micro-orifices is adapted to receive a single cell therein, and such that the at least one chamber includes a first region on one side of the membrane, and a second region on another side of the membrane; a delivery device in fluid communication with the first region of the at least one chamber, the delivery device being adapted to deliver a fluid to the first region; and a removal device in fluid communication with the second region of the at least one chamber, the removal device being adapted to remove a fluid from the second region.

In a further example embodiment, this invention provides a device comprising: a housing defining at least one chamber therein; a plurality of membranes, each of the membranes defining a plurality of micro-orifices and being configured such that each of the plurality of micro-orifices is adapted to receive a single cell therein, the membranes being disposed in the at least one chamber such that the at least one chamber includes a first region on one side of the membranes, and a second region on another side of the membranes; a delivery device in fluid communication with the first region of the at least one chamber, the delivery device being adapted to deliver a fluid to the first region; and a removal device in fluid communication with the second region of the at least one chamber, the removal device being adapted to remove a fluid from the second region.

In one example embodiment, this invention provides a device comprising: a housing defining at least one chamber therein; a means for controlling fluid flow disposed in the at least one chamber and defining a plurality of micro-orifices, the means for controlling fluid flow being configured such that each of the plurality of micro-orifices is adapted to receive a single cell therein, and such that the at least one chamber includes a first region on one side of the means for controlling fluid flow, and a second region on another side of the means for controlling fluid flow; a fluid delivery means in fluid communication with the first region of the at least one chamber, the fluid delivery means being adapted to deliver a fluid to the first region; a fluid removal means in fluid communication with the second region of the at least one chamber, the fluid removal means being adapted to remove a fluid from the second-region.

In a further example embodiment, this invention provides a microfluidic network, said network being adaptable for integration with a device for coculturing on a cell culture support surface of the device, said network comprising: a plurality of channels, the channels being adapted to deliver at least one agent to the cell culture support, and a removal device, the removal device being adapted to remove at least one analyte from the cell culture support.

In yet another example embodiment, this invention provides a method of analyzing an effect of candidate compound on a cellular coculture, said method comprising: a) coculturing at least two different cell types in a two-dimensional coculture device; b) contacting at least one cell type with a therapeutically effective dose of at least one test compound for a therapeutically effective time period; c) removing at least one analyte of the coculture; and d) performing an assay on the at least one analyte.

### DESCRIPTION OF THE DRAWINGS

FIG. 1A a illustrates a single test chamber. The arrows indicate the flow of the test compound (starburst) through the test compound delivery device into the test chamber. The test compound may: (1) kill or decrease the viability of the test cell; (2) be metabolized or chemically altered by the test cell; (3) pass through the test cell unchanged, or be; (4) unreleasably absorbed by the test cell (not shown). The magnified view to the right is a close up view of the seal formed between the test cell and the patterning membrane which prevents paracellular flow of the test compound into the analyte. Once the analyte collects downstream of the patterning membrane the analyte removal device transfers the analyte for examination. FIG. 1B shows a plurality of test chambers. Such an array may have the same size and pitch of a standard 96-, 384- or 1536-well microtiter dish.

FIG. 2 shows multiple patterning membrane configurations of the test chamber. The illustration shows a device having one (A), two (C), or three (B) patterning membranes. FIG. 2D shows a test chamber with a filter membrane downstream of a patterning membrane.

FIG. 3 illustrates a trans configuration of a single test chamber (101) for vertical flowing of compounds for screening assays which comprises a test compound delivery device (102), one or more patterning membranes (103) having one or more test cells (104) immobilized therein, and an analyte removal device (105) which removes fluid that has passed through the test cell in the collection chamber (106). A filter membrane (107) may be positioned downstream of the patterning membrane(s) and upstream of the analyte removal device. Preferably, the filter membrane is of a porous nature having micropores (108) that are small enough to block passage of the test cell through it.

FIG. 4 illustrates a cis configuration of a single test chamber for horizontal flowing of compounds for screening assays.

FIG. 5 schematically depicts microcontact printing and membrane patterning techniques for arraying single cells over a large area.

FIG. 6 illustrates the assembly of the porous sheet and the elastomeric membrane configured as an insert for a well of a plate, e.g., 24-well, 96-well or greater number of wells.

FIG. 7 illustrates the format and modularity of the absorption assay device.

FIGS. 8A-8B: FIG. 8A schematically represents the co-culture of hepatocytes and fibroblasts. FIG. 8B is a fluorescence image representing endothelial cells surrounded by fibroblasts to demonstrate the feasibility of the co-culture device and assay.

FIG. 9 illustrates the integration of the metabolism and absorption assays into one assay device. Not shown are secondary outlet channels for sampling fractions from each individual assay well. Substances are transferred between wells by gravitational flow and diffusion.

FIG. 10 depicts a Hepatocyte Biosensor for toxins and viruses.

FIG. 11 depicts one embodiment of an absorption microassay.

FIG. 12 depicts one embodiment of an metabolism microassay.

FIGS. 13A-13C: FIG. 13A illustrates a continuous and contiguous coculture of two different cell types. FIG. 13 B illustrates coculture of two different cell types in which the cells are separated in individual islands. FIG. 13C shows a matrix of variable height which may be used to surround/cover cultured cells to determine the motility of the cells, as well as ability of the cells to burrow through the matrix.

FIG. 14 depicts coculture of two cell types, wherein the two cell types are separated by a channel that may be opened via a valve at any time during the coculture to expose the first cell type to the metabolic products or secretions of the second cell type. Alternatively, the channel may have a filter disposed therewithin to capture a substance, e.g., a drug.

FIG. 15 shows various valves which may be integrated into channels of a coculture device. (1) Valves may include magnets that attach to metal beads to close the channel. (2) Pressure may be applied mechanically or by gravitation to close valves having structures that fit into each other to form a seal. (3) A valve may include a combination of magnet and metal beads and structures that fit into each other to form a seal.

FIG. 16 illustrates flexible formats for bioassay devices. Formats may be used to study motility and spreading of cells, co-culture of cells, cell differentiation, chemotaxis, cellular invasion, e.g., into a matrix, and adhesion/rolling of cells in one device.

FIG. 17 depicts Cell Mosiac™ Assays for motility. Cells are deposited in a plurality of microwells, wherein each microwell has a patterned mask to permit growth within the pattern formed thereby, and cells spreading may be monitored once the mask is peeled off.

FIGS. 18A-18B shows a CMA or co-culture device in top view (FIG. 18A) and side view (FIG. 18B). Electrode 1 is in contact with the gold. Electrode 2 is in solution. Cell type 1 is plated on the glass. The potential applied to damage the EG SAM des not affect those cells because they are adhered to insulated areas (glass patches).

FIG. 19 illustrates a co-culture patterning surface which has built-in electrodes, therefore there is no need for stenciling membranes.

FIG. 20 shows a co-culture patterning surface with electrodes, which permits plating multiple and different cell populations. Electrodes are isolated by thin glass strips (1 micron or less) to which cell will not attach for lack of space; the electrodes are activated in sequence.

FIG. 21 illustrates EG+HDT SAMs approach. In order to ensure that cell type 1 population will not be detached after application of the potential, a longer chain than HTD can be used (like C24). The longer chain may be more stable with respect to the applied potential.

FIGS. 22A-22B depict culturing cells on EG SAM. T47 D cells were cultured on glass in areas separated by gold-coated areas presenting an ethylene glycol terminated SAM. The cells were cultured for 24 hours (images in the left columns) before applying a bias of 600-1300 mV. After one day in culture the cells began to migrate out of the glass surface onto the SAM surface; the images in the right columns were taken three days after applying the voltage. The migration out of the pattern is not caused by natural degradation of the SAM, because it has been shown that these cells can be maintained in a pattern separated by EG groups for more than one week

FIGS. 23A-23D show a transmigration (extravasation) device. The device is shown in chip layers (FIG. 23A), as assembled (FIG. 23B), and the bottom layer (FIG. 23C). The top layer (109) has an outlet well (112), an inlet well (113) and a cell culture well (115). The middle layer (110) is a porous membrane. The bottom layer (111) has an outlet receptacle (112 A) and an inlet receptacle (113A) linked by a linear and planar microchannel network (114). FIG. 23D shows a cross-sectional view of an alternate transmigration/extravasation device. The top and bottom layers are made from PDMS and a membrane is disposed between the two layers; the membrane may be any thin sheet having pores of appropriate sizes. The top layer has a microchannel network on its surface; the network may be integrated as part of the device or may be a separate device which is placed thereon.

FIG. 24 depicts two cell patterning techniques. In FIG. 24 A, the “CMA” corresponds to the use of stencil membranes and the “Echem” corresponds to electrochemical patterning. The graph of FIG. 24B shows the results obtained by measuring the cell “island” size over time. After the cells were patterned, and the constraints removed (either the membrane or the SAM was released in the electrochemcial patterning method), the cells grew and spread across the support.

FIG. 25 depicts an experiment where endothelial cells are patterned islands and allowed to grow to confluence (FIG. 25A). After electrochemical stimulation, cancer cells (the small dots) were seeded onto the areas surrounding the endothelial islands (FIG. 25B). The cells were stained so VE-Cadherin, which is present in cell-cell junctions is seen as lighter grey lines between the cells.

FIG. 26 is a close up view of the cells in FIG. 25. As seen in the picture labeled “co-culture,” the cancer cells (the small light colored cells) are invading the endothelial cells. The endothelial cell island has decreased in size and the intensity of the staining of VE-Cadherin has decreased, thus indicating that the cancer cells have invaded the cell-cell junctions.

FIG. 27 shows results of an experiment where control cells (HUVEC epithelial cells) were patterned and allowed to grow to confluence (FIG. 27A). Three different cell types were co-cultured with the HUVEC cells: MCF-10A (normal breast epithelium)(FIG. 27B); MCF-7 (noninvasive breast cancer line (FIG. 27C); MDA-MB-231 (invasive breast cancer line)(FIG. 27D). The figures show that over time, invasive cells lead to dissolution of cell-cell junctions, as indicated by the fact that the original cell boundary of the island has disappeared and by the fact that the cancer cells have invaded into the cell-cell junctions. These figures also demonstrate that the size of the island decreases in proportion to the invasiveness of the cells.

FIG. 28 includes three pictures of control slides. The nucleus in seen as a light grey round portion within the cells. The cadherins are seen as the light grey lines between the cells.

FIG. 29 are two slides taking from an experiment where HUvEC cells were co-cultured with MCF10a (non-cancer breast epithelial cells).

FIG. 30 shows the results of co-culturing HUVEC cells with MDA-MB-231 (invasive breast cancer). The cancer cells are seen to invade the HUVEC cells (invading and disrupting the cell-cell junction) and destroying the originally patterned island conformation.

FIG. 31 shows the results of co-culturing HUVEC cells with MCF10a (non-cancer breast epithelial cells). This shows that non invasive cells do not effect the integrity of the endothelial cell islands and cell-cell junctions.

FIG. 32 depicts co-culturing of HUVEC cells with MCF-10a cells and with MDA-MB-231 cells. This figure shows that the non-invasive cells do not disrupt the island boundary where as the invasive cancer cells do.

FIG. 33 depicts co-culturing, but in this example, cancer cells were first patterned into islands and then normal epithelial cells were plated around the cancer cell islands. The cells were monitored for invasion of cancer cells into the epithelial cells. As demonstrated in the figure, invasive cells entered into the epithelial cells whereas non-invasive cells did not.

FIG. 34 is a close up view of FIG. 33.

FIG. 35 shows the results of an experiment where HUVEC endothelial cells were plated. An electrochemical stimulus was applied to remove the SAMs and then either MCF10a or MDA-MB-231 were patterned around the HUVEC cells. After two hours, the invasive cancer cell line has invaded the HUVEC cells and disrupted the island shape and entered into the cell-cell junctions.

FIGS. 36-38 depict results of three experiments where three different compounds were assayed for their ability to affect cell motility and/or cell invasion using the methods and devices of the present invention. Cell motility was measured by measuring cell movement of cancer cells that were not surrounded by normal endothelial cells. Cell invasion was measured by measuring cell movement where the cancer cells were surrounded by normal cells. In FIG. 36, the test compound seems to be more effective on cell motility than cell invasion. In FIG. 37, the test compound seems to be more effective on cell invasion than cell motility. In FIG. 38, the test compound effects cell invasion and has hardly any effect on cell motility. These figures demonstrate the importance of measuring both cell motility and invasion and that drugs may not effect each movement in the same way.

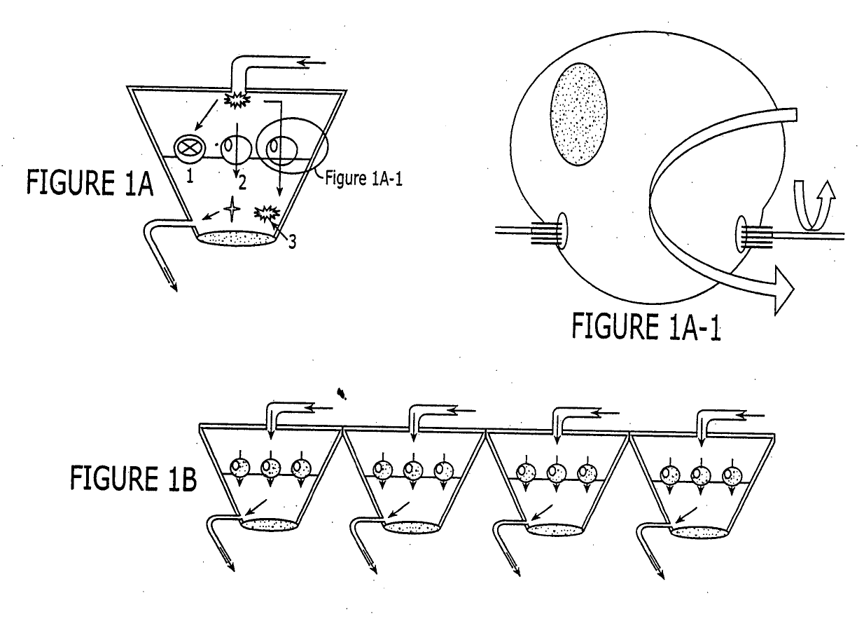

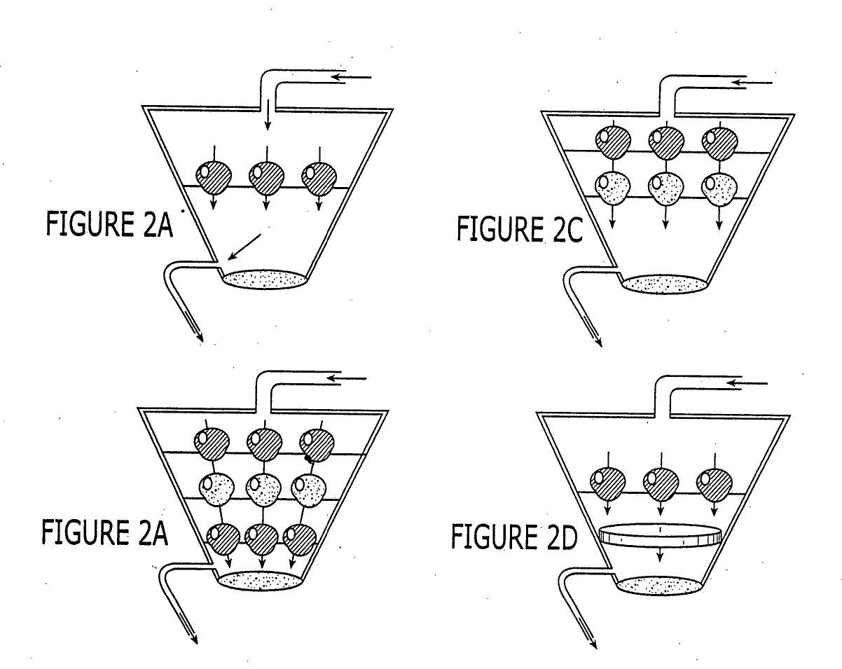

### DETAILED DESCRIPTION

The invention provides for a set of high-throughput, flexibly formatted, cell-based assays for drug absorption, permeability, metabolism, excretion and toxicity studies that are highly biologically relevant and precise. The inventors have further determined that by linking fluid paths between these various cell-based formats, one creates a system that nearly mimics the fate of a compound as it passes through an organism. The present invention provides high-throughput in vitro system that models essential parts of the processes of absorption, metabolism and excretion. Furthermore, this system enables the simultaneous determination of the toxic effects of compounds and their metabolites on several different cell types. The present invention presents advanced cellular assays that reproduce these biological processes in mixed cell culture systems with nearly biological environments and integrates them in a format compatible with high-throughput screening. This invention provides a major step toward developing predictive in vitro models for human response to therapy, including adverse effects to drugs, organ-specific toxicity, accumulation of drug metabolites, and PK/PD characterization. The invention can simulate in vivo systems such as, but not limited to, immune and inflammatory response, endocrine functions, and central nervous system (CNS).

The present invention will increase productivity by increasing success rates when pre-screened compounds reach the animal-study phase and will contribute to drug safety by providing an additional set of data on drug safety and kinetics. In addition to applications in drug testing, the present invention provides technology that (including assays and devices) enable phenotypic cloning in mammalian systems, assists in cancer cell characterization for optimal chemotherapy, and facilitate the identification of the ligands for orphan receptors. The invention further provides a basic platform as a biosensor in the generation of in vitro systems that can determine the effects of and predict the body's reaction to previously uncharacterized drugs, chemical hazards, and toxins, including those that may be encountered on the battlefield.

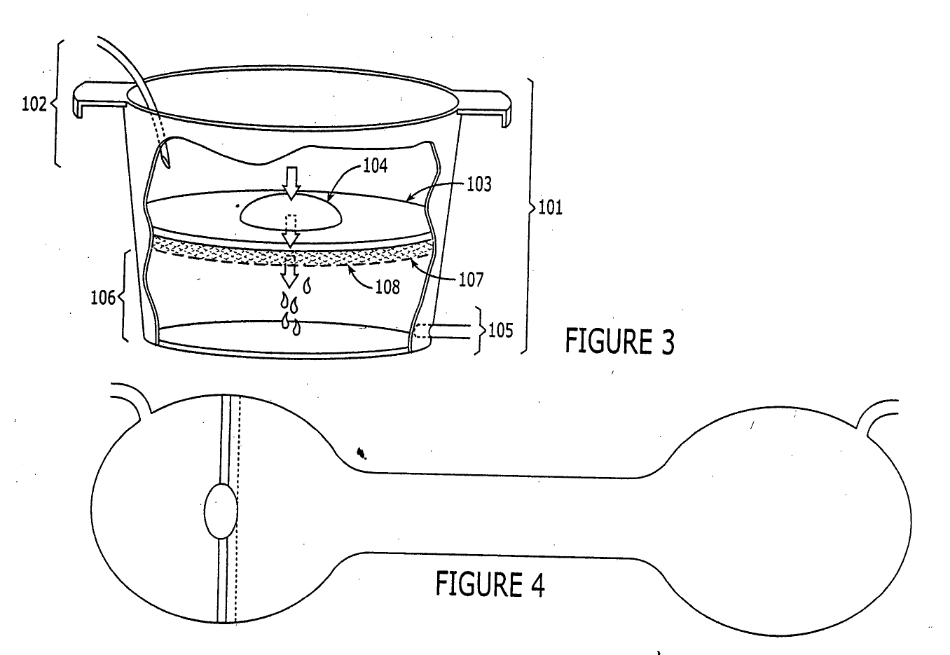

Specifically, the present invention provides assay devices that analyze the absorption, permeability and/or metabolism of a candidate compound by a cell, and methods of use thereof. The invention has one or more test chambers (101) which include a test compound delivery device (102), one or more patterning membranes (103) having one or more test cells (104) immobilized thereon, and an analyte removal device (105). The flow of the test compound through the test chamber may be in horizontal, i.e., the test chamber is in a cis-configuration (FIG. 4), or the flow may be vertical, i.e., the test chamber is in a trans-configuration (FIG. 3).

“Analyte” as referred to herein is liquid and/or media that has passed directly through the test cell or secreted by the test cell. Analyte may or may not contain test compound and/or metabolites thereof, as well as other nucleic acids, polypeptides, and molecules that may serve as markers of test cell viability and functionality.

A test compound delivery device delivers a test compound to a patterning membrane (104) having one or more test cells immobilized therein. A test compound will traverse the patterning membrane only if it passes directly through a test cell. The compound may interact with a test cell in any combination of four ways: 1) a test compound may kill or decrease the viability of the test cell; 2) a test compound is absorbed by a test cell; 3) a test compound passes through a test cell unaltered; or 4) a test compound is metabolized by a test cell and released in a chemically altered state. The invention provides an analyte removal device (105) that removes fluid that has passed through a test cell collected in a collection chamber (106). This fluid may then be examined for the presence of a test compound or any metabolites derived from it.

A “test compound” as defined herein refers to a chemical, nucleic acid, polypeptide, amino acid or other compound which is applied to a test cell immobilized on a patterning membrane(s). The object of the invention is to rapidly determine the extent to which and the rate at which a test compound is absorbed, is permeable, and/or is metabolized by a test cell. The invention further allows the isolation and subsequent examination of an analyte, comprising either a test compound having flowed-through a cell and/or test compound metabolites. Examples of test compounds include, but are not limited to, drug candidates, such as derived from arrays of small molecules generated through general combinatorial chemistry, as well as any other substances thought to have potential biological activity. A test compound is applied to a cell culture membrane and/or patterning membrane by a test compound delivery device (102) such that the test compound is absorbed, flowed through and/or is metabolized by the test cell. A test compound may be labeled such that it and/or its metabolites are easily detected in subsequent analysis. For example, test compounds may be synthesized using radioactive isotopes fluorescent tags.

A “test compound delivery device” (102) as defined herein refers to devices, apparatuses, mechanisms or tools that are capable of delivering test compounds to a patterning membrane(s). Examples of test compound delivery devices include but are not limited to pipettes or robotic devices well known in the art such as Tecan, PlateMate, or Robbins. Preferably, a test compound delivery device is a microfluidic device that delivers a solublized test compound to a test chamber, and specifically to contact immobolized cells on a patterning membrane.

A microfluidic device as described herein refers to a surface into which micro channels are fabricated as those disclosed by U.S. Pat. No. 6,048,498, which is hereby incorporated by reference in its entirety. Preferably, the microfluidic device is made of any material such as glass, co-polymer or polymer, most preferably urethanes, rubber, molded plastic polymethylmethacrylate (PMMA), polycarbonate, polytetrafluoroethylene (TEFLON™), polyvinylchloride (PVC), polydimethylsiloxane (PDMS), polysulfone, and the like. Such materials are preferred for their ease of manufacture, low cost and disposability, as well as their general inertness to most extreme reaction conditions. Such devices are readily manufactured from fabricated masters, using well known molding techniques, such as injection molding, embossing or stamping, or by polymerizing a polymeric precursor material within the mold. Soft lithography techniques known in the art are preferably used. See Love, et al., MRS Bulletin, pp. 523-527 (July 2001) “Fabrication of Three-Dimensional Microfluidic Systems by Soft Lithography,” Delamarche et al,: Journal of American Chemical Society, Vol. 120, pp. 500-508 (1998), Delamarche et al,: Science, Vol. 276, pp. 779-781 (May 1997), Quake et al., Science, Vol. 290, pp. 1536-1540 (Nov. 24, 2000), U.S. Pat. No. 6,090,251, all of which are hereby incorporated by reference. A microfluidic device may be fabricated by other known techniques, e.g., photolithography, wet chemical etching, laser ablation, air abrasion techniques, injection molding, or embossing. When a microfluidic device is mated to a test chamber, channels flow a test compound containing liquid by either capillary action, positive pressure or vacuum force. The diameter of the channels of a microfluidic device should be large enough to prevent clogging of the channel. Further, channels may be coated with various agents to prevent nonspecific absorption of a test compound or its metabolites.

A “patterning membrane having one or more test cells immobilized therein” as defined herein refers to any preferably substantially flat surface having micro-through-holes in which one or more test cells are immobilized and/or arrayed in a uniform pattern. Preferably, one test cell is immobilized in each micro-through-hole. The size of each micro-through-hole depends on the size of the test cell to be employed. Preferably the diameter of each micro-through-hole is smaller than that of the test cell so that the test cell rests in but cannot slide through the each micro-through-hole. The size of the each micro-through-hole should be from about 10 to about 50 microns. Preferably, a patterning membrane is made of a material such as glass, co-polymer or polymer, most preferably urethanes, rubber, molded plastic, polymethylmethacrylate (PMMA), polycarbonate, polytetrafluoroethylene (TEFLON™), polyvinylchloride (PVC), polydimethylsiloxane (PDMS), polysulfone, and the like. Such membranes with each micro-through-holes are readily manufactured from fabricated masters, using well known molding techniques, such as injection molding, embossing or stamping, or by polymerizing the polymeric precursor material within a mold. Standard soft lithography techniques are preferably used to fabricate a substrate. See Love, et al., MRS BULLETIN, pp. 523-527 (July 2001) “Fabrication of Three-Dimensional Microfluidic Systems by Soft Lithography,” Delamarche et al,: JOURNAL OF AMERICAN CHEMICAL SOCIETY, Vol. 120, pp. 500-508 (1998), Delamarche et al,: SCIENCE, Vol. 276, pp. 779-781 (May 1997), Quake et al., SCIENCE, Vol. 290, pp. 1536-1540 (Nov. 24, 2000), U.S. Pat. No. 6,090,251, all of which are hereby incorporated by reference. Such membrane materials are preferred for their ease of manufacture, low cost and disposability, as well as their general inertness to most extreme reaction conditions.

In order to prevent test compound movement through membrane micro-through-holes without passing directly through a test cell, i.e., paracellular flow, a pattering membrane may have treated surfaces, such as, derivatized or coated surfaces, to enhance the test cell's ability to form a test compound impermeable seal between the membrane and the test cell. In one embodiment, the membrane may be coated with junction forming proteins to stimulate the formation of seals having adherens junctions and/or tight junctions. In a further embodiment a patterning membrane may be coated with extracellular matrix and/or basal lamina components such as RGD-containing peptides, laminins, collagens, fibronectins and the like to stimulate integin binding and the formation of hemidesmosomes and focal contacts between the test cell and the patterning membrane. In yet another embodiment, an agent may be introduced that targets test cell-patterning membrane interfaces and that can be polymerized under conditions not damaging to the test cell to create a solid to which the cells adhere and form a seal that does not allow test compounds to pass through the patterning membrane unless it passes through the cells.

A “test cell” as defined herein refers to one or more cells immobilized in the pattering membrane. A single cell is positioned in each patterning membrane micro-through-hole such that test compounds are not able to traverse the patterning membrane without moving through the test cell itself. The amount of movement through and/or metabolism of the test compound by the test cell over time determines the extent to which and the rate at which, a test compound is absorbed and/or is metabolized by the test cell. In order to prevent test compound movement through the membrane micro-through-holes without passing directly through the test cell, the pattering membrane may be treated to enhance the test cell's ability to form a seal between the membrane and the test cell. As stated above, the membrane may be coated with junction forming proteins to stimulated the formation of adherens junctions and tight junctions. Additionally, the test cells may be genetically modified to overexpress ectopic junction forming proteins including but not limited to cadherins and claudin-1, ZO-1, and occludin to induce the formation of adherens junctions and/or tight junctions, respectively, between themselves and the patterning membrane. In a further example, where the patterning membrane may be coated with extracellular matrix and/or basal lamina components such as RGD-containing peptides, laminins, collagens, and fibronectins, the test cells may be genetically modified to overexpress ectopic proteins such as integrins to further reinforce the formation of hemidesmosomes and focal contacts between the test cell and the patterning membrane.

A test cell may be derived from any cell lineage derived from a test compound target tissue. A target tissue is one with which a particular test compound, i.e., putative drug, is thought to come into physiological contact in vivo. Physiological contact refers to whether a cell type is thought to absorb, metabolize, or be permeable to a test compound in vivo.

One embodiment of the invention utilizes Caco-2 cells, which are derived from a colonic tumor cell line. Caco-2 test cells spontaneously exhibit enterocyte-like characteristics when cultured. Given the difficulties in maintaining long-lasting cultures of enterocytes and the fact that Caco-2 cells have low paracellular permeability, Caco-2 test cells provide an excellent model suitable for carrying out analysis of absorption, metabolism and toxicity of test compounds on the gut lining. Artursson et al., Advanced Drug Delivery Reviews, 46 (2001) 27-43, herein incorporated by reference in its entirety. Preferably, the Caco-2 test cells form tight junctions with the patterning membrane, because tight junctions restrict the movement of drugs between cells (paracellular movement) of the gut lining in vivo. Accordingly, it is important that test compounds do not move into the analyte between the test cells and the patterning membrane as to obfuscate examination of an analyte. To achieve this, Caco-2 test cells may be genetically modified to overexpress ectopic junction forming proteins including but not limited to cadherins and claudin-1, ZO-1, and occludin to induce the formation of adherens junctions and/or tight junctions, respectively, between themselves and the patterning membrane. The membrane may also be coated with these junction forming proteins to stimulate the formation of adherens junctions and tight junctions. In this embodiment, the investigator will be able to determine if a test compound passes through an enterocyte i.e., the gut lining, and if so at what rate. Further, the invention provides the means to determine to what extent that test compound is metabolized by the cells of the gut lining, and the toxicity of the test compound on the enterocyte.

Another embodiment of this aspect of the invention enables one to rapidly screen test compounds thought to potentiate chemotherapeutics targeted to multi drug resistant tumors. Caco-2 test cells express high levels of P-glycoprotein (P-gp), which plays an important role in determining drug disposition and contributing to multi drug resistance (MDR). It has been shown that a good correlation exists between the in vitro test compound flux ratio across a monolayer and in vivo P-gp. Adachi, et. al., Pharm Res 2001 December;18(12):1660-8. Collectively, determining the in vitro test compound flux across the P-gp-expressing Caco-2 cells test cell into the collection device may be used to predict in vivo P-gp function. Additionally, the extent of ATP-hydrolysis in the test cell may also be a useful parameter for in vivo prediction, particularly when screening for test compounds that induce ATP-depletion. For example, certain drugs have the ability to inhibit P-glycoprotein and sensitize MDR cells to chemotherapeutics, which appears to be a result of ATP depletion. Because many mechanisms of drug resistance are energy dependent, a successful strategy for treating MDR cancer could be based on selective energy depletion in MDR cells. Batrakova et al., Br J Cancer 2001 December;85(12):1987-97. Therefore, screening for energy-depleting effects of test compounds on Caco-2 test cells, provides an excellent tool in searching for drugs meant to fight cancer by increasing chemotherapeutic sensitivity. Following exposure of the Caco-2 test cell with various test compounds, the test cells and analyte can be examined for ATP and ADP levels by assays known in the art such as the chemoluminescence luciferin-luciferase assay.

In another embodiment of this aspect of the invention, the test cells are hepatocytes. Traditionally, it has been difficult to maintain hepatocytes in monoculture. Co-cultures of hepatocytes, with another cell type have been recognized to prolong cell survival rates, maintain phenotype, and induce albumin secretion in hepatocytes. Such co-cultures have been limited by the inability to manipulate or control the interaction of the two cell types in the culture. Generally, to prepare conventional co-cultures, cells of one type are seeded onto a substrate and allowed to attach; cells of a second type then are seeded on top of or next to the cells of the first type. See Bhatia, S. N., et al. U.S. Pat. No. 6,221,663 herein incorporated by reference. In such co-cultures, parameters such as cell number are controllable, but the spatial orientation of cells within the co-culture is not controlled (Element, B., et al. “Long-Term Co-Culture of Adult Human Hepatocytes with Rat Liver Epithelial Cells: Modulation of Albumin Secretion and Accumulation of Extracellular Material” Hepatology 4(3): 373-380 (1984). An embodiment of the invention provides control of the spatial orientation through immobilization of one or more hepatocyte test cells on a patterning membrane, preferably one hepatocyte per each micro-through-hole. Following hepatocyte test cell immobilization, fibroblasts are seeded around each test cell. Most preferably, each micro-through-hole has a single test cell and about 3 to about 4 fibroblasts seeded around each hepatocyte test cell, forming a hepatocyte patch. Preferably, a hepatocyte patch is about 75 to about 150, most preferably about 85 to about 125 microns in diameter and spaced about 100 to about 500, most preferably about 250 to about 350 microns apart. Such an arrangement maintains hepatocyte cell viability for 2 or more months. The spatial orientation is preferably accomplished by soft lithography techniques to achieve desired arrays of cells. In order to facilitate this fibroblast arrangement, the area around the surface of the patterning membrane around the micro-through-hole may be treated to facilitate fibroblast adhesion. Such treatments may include but are not limited to coating with poly-L-lysine, laminin, and fibronectin. Additionally, the invention provides immobilizing one hepatocyte per micro-through-hole yet having multiple hepatocytes expressing various isoforms of cytochrome P450 on a given patterning membrane within the test chamber. Preferably the isoforms are CYP 3A4, 2B6 and 2C9. Such an arrangement yields a test chamber whose test cell population more accurately reflects the cytochrome P450 expression relevant to drug metabolism of the actual liver.

Using hepatocytes as test cells, the invention enables one to determine whether a test compound passes through a hepatocyte; and, if so, at what rate. Further the invention enables one to determine the extent that the test compound is metabolized by the cells of the liver, and if cytochrome P450 is involved. In addition, the toxicity of the test compound on the hepatocyte can be determined by observing the health and viability of the cells exposed to the test compound. Further, since the present invention, by using a novel co-culture ratio of hepatocytes and fibroblasts, allows the hepatocytes to remain variable for a long time period, long term effects on drug doses can be studied. In the past, since hepatocytes could not be maintained in culture for long periods of time; high doses or single doses could only be studied. Unfortunately, many of the nefarious effects of drugs and other chemical therapies are only realized after long term administration. Examining the analyte after it has passed through the hepatocyte further allows the monitoring of hepatocyte metabolism and viability by assaying such markers as albumin secretion, urea secretion cytochrome P450 activity and inducibility, glutathione-5-transferase expression and activity.

Other exemplary embodiments of this aspect of the invention include, but are not limited to, a test cell derived from a particular tumor cell line, if the test compounds are putative anticancer agents specific for that or other tumor cells. Additionally, central nervous system derived cells may be used as test cells if the test compounds are to be tested for blood-brain-barrier permeability. To reiterate, the test cell may be derived from any cell lineage for which a particular test compound, i.e. putative drug, is thought to come into physiological contact in vivo. Physiological contact refers to whether a cell type is thought to absorb, metabolize, or be permeable to a test compound in vivo.

An “analyte removal device” as define herein refers to devices, apparatuses, mechanisms or tools that are capable of removing analyte. Examples of analyte removal device include but are not limited to pipettes or robotic devices well known in the art such as Tecan, PlateMate, or Robbins. Preferably, the analyte removal device is a microfluidic device that removes the solublized test compound and/or its metabolites to the patterning membrane. A microfluidic device as described herein refers to a surface into which channels are fabricated as those disclosed by U.S. Pat. No. 6,048,498, which is hereby incorporated by reference in its entirety. Preferably, the microfluidic device is made of any material such as glass, co-polymer or polymer, most preferably urethanes, rubber, molded plastic polymethylmethacrylate (PMMA), polycarbonate, polytetrafluoroethylene (TEFLON™), polyvinylchloride (PVC), polydimethylsiloxane (PDMS), polysulfone, and the like. Such devices are readily manufactured from fabricated masters, using well known molding techniques, such as injection molding, embossing or stamping, or by polymerizing a polymeric precursor material within the mold. Soft lithography techniques known in the art may also be used. See Love, et al., 2001; Delamarche et al, 1998; Delamarche et al, 1997; Quake et al., 2000. Such materials are preferred for their ease of manufacture, low cost and disposability, as well as their general inertness to most extreme reaction conditions. The microfluidic device is fabricated by known techniques, e.g., photolithography, wet chemical etching, laser ablation, air abrasion techniques, injection molding, or embossing. When the analyte removal microfluidic device is mated to the test chamber, channels flow solublized test compound and/or its metabolites by either capillary action, positive pressure or vacuum force out of the test chamber. The diameter of the channels of the microfluidic device should be large enough to prevent clogging of the channel.

Upon removal of analyte by the analyte removal device, its contents may be examined to ascertain the presence of the test compound and/or metabolites thereof by any of the common techniques known in the art. For example, the analyte may be examined using standard chromatographic techniques including ion-exchange, size-exclusion, affinity, gel, high pressure-liquid chromatography, thin-layer chromatography, sequential extractions, counter-current chromatography, hydrophobic interaction chromatography, hydrophilic interaction chromatography and/or other chromatography techniques, as well as scintillation counters, Mass-spectroscopy NMR or IR analysis, bioluminescence, UV absorption analysis and all other techniques useful for identifying and characterizing polypeptides, nucleic acids and small molecules and/or their metabolites.

In another aspect of the invention, the device has more than one pattern membrane to simulate multiple physiological contacts between the test compound and cells in vivo. For example, a drug may be efficiently taken up through the gut lining but then metabolized in the liver, such that the drug is inactivated before it reaches its target tissue. Alternatively, it is of great importance to know, for example, how and if the cells of the gut lining chemically alter the drug resulting in metabolites and what, if any, are the potentially toxic effects of these metabolites to other downstream cells e.g. hepatocytes.

One embodiment of the invention provides a first pattering membrane having Caco-2 test cells, preferably one Caco-2 cell per micro-through-hole, which spontaneously exhibits enterocyte-like characteristics when cultured. This embodiment further provides a second downstream pattering membrane having one or more hepatocyte test cells, preferably one hepatocyte per each micro-through-hole with fibroblasts seeded around each test hepatocyte cell. Most preferably, each micro-through-hole has a single test cell and about 3 to about 4 fibroblasts seeded around each hepatocyte test cell. This embodiment simulates the biological path taken by many drugs through the body and allows the investigator to determine to what extent a test compound that has passed through Caco-2 cell is processed by the cells of the liver. In this embodiment, the presence of the test compound and/or its metabolites in the analyte may provide pharmacokinetic information with respect to drug clearance and potential effect on liver cells. The hepatocyte test cell and resulting analyte can also be assayed for liver cell function by measuring albumin secretion, urea secretion cytochrome P450 activity and inducibility, glutathione-5-transferase expression and activity, ZO-1 expression, and/or gap-junction detection. In a further example, the invention provides adding more than one test compound to ascertain whether or not there exits the potential that one test compound that acts as a potent inhibitor of a CYP450 enzyme leads to undesirable drug-drug interactions when administered with another test compound or another drug that interacts with the same CYP450.

Another embodiment of the invention provides a first pattering membrane having one or more hepatocyte test cells, preferably one hepatocyte per each micro-through-hole with fibroblasts seeded around each test hepatocyte cell. Most preferably, each micro-through-hole has a single test cell and about 3 to about 4 fibroblasts are seeded around each hepatocyte test cell. This embodiment further provides for a second pattering membrane having a test cell, preferably one cell per micro-through-hole, which is derived from the putative test compound target tissue. This embodiment enables one to determine to what extent a test compound is modified by the test cell hepatocyte in the first patterning membrane and to what extent the test compound and/or its metabolites are absorbed, further metabolized or toxic to the target tissue test cell. This assay further models a drug's physiological contacts in vivo because following uptake in the gut, a drug must survive oxidative modifications in the liver before it get to the desired site (e.g., target organ or primary tumor). As such the invention provides a device for assaying the affect of a liver metabolized drug on its target tissue.

In yet another embodiment of this aspect of the invention, the device has three pattern membranes to simulate multiple physiological contacts between the test compound and cells in vivo. In this embodiment of the invention, there is provided a first pattering membrane having Caco-2 test cells, preferably one Caco-2 cell per micro-through-hole, which spontaneously exhibits enterocyte-like characteristics when cultured. This embodiment further provides a second downstream pattering membrane having one or more hepatocyte test cells, preferably one hepatocyte per each micro-through-hole with fibroblasts seeded around each test hepatocyte cell. Most preferably, each micro-through-hole has a single test cell and about 3 to about 4 fibroblasts seeded around each hepatocyte test cell. This embodiment further provides for a third downstream pattering membrane having a test cell, preferably one cell per micro-through-hole, which is derived from the putative test compound target tissue. This assay further models a drug's physiological contacts in vivo because it models the test compound uptake in the gut, liver oxidative modification, and effect on the desired site cell type (e.g., target organ or primary tumor). As such the invention provides a device for assaying the affect of a liver metabolized drug on its target tissue.

In another embodiment of the invention, there is provided a first pattering membrane having one or more CNS cells, preferably one CNS per each micro-through-hole, to simulate a blood-brain-barrier. This embodiment further provides a second downstream pattering membrane with test cells derived from a CNS target cell type that may potentially be sensitive to metabolites of the test compound.

In another aspect of the invention a filter membrane (107) is positioned downstream of the patterning membrane(s) and upstream of the analyte removal device. In one embodiment of this aspect of the invention, the filter membrane blocks the inadvertent uptake of the test cell by the analyte removal device. This embodiment provides a filter membrane made of any material such as glass, co-polymer or polymer, most preferably urethanes, rubber, molded plastic polymethylmethacrylate (PMMA), polycarbonate, polytetrafluoroethylene (TEFLON™), polyvinylchloride (PVC), polydimethylsiloxane (PDMS), polysulfone, and the like. Preferably, the filter membrane is of a porous nature having micropores (108) that are small enough to block passage of the test cell through it. Preferably, the micropores are less than about 5, most preferably less than about 2 microns in diameter.

In another embodiment of this aspect of the invention, the filter membrane is capable of allowing the passage of select molecules. The filter membrane may be selected such that it only allows the passage of molecules of a certain size in a manner similar to size-exclusion chromatography. This is useful if the test cell naturally secretes molecules of various sizes that obfuscate the examination of the analyte. Molecules above a particular size are thus screened from the analyte before it is removed by the analyte removal device.

The invention may have one or more test chambers which include a test compound delivery device, one or more patterning membranes having one or more test cells immobilized therein, and an analyte removal device. Preferably, the test chamber is on a scale such that it can be fitted into the well of a standard 96-well, 384-well, or 1536-well microtiter dish. It is also preferable that the test chambers be attached to one another either as strips or grids to be rapidly inserted and removed from a microtiter plate. The small size of the test chambers allow the investigator to assay a large number of test compounds concurrently utilizing only a few microliters from often limited sources of solublized test compounds.

The present invention provides high throughput, precise, flexibly formatted, cell-based assay devices and methods for drug absorption, metabolism and toxicity that are highly biologically relevant, e.g., to predict the human body's interaction with test compounds so as to enable development and testing of therapeutic drugs, as well as to identify the presence of biological agents including toxins and pathogens. The assays discussion below are illustrative of the assays of the present invention, and are not intended to limited the scope of the invention.

For a drug to be available after oral administration, it must first pass through the lining of the gastrointestinal tract and penetrate into the blood stream. Several transport mechanisms across the gastrointestinal tract have been identified. The most prevalent route of drug absorption is passive transcellular diffusion: unassisted drug traffic through the membranes of the cell. In active transport, compounds are pumped into and out of the cells by specialized proteins called P-glycoproteins (PGP). Compounds often also diffuse through the junctions between cells—paracellular transport. Successful in vitro assays must be able to differentiate among these three modes of absorption.

The most widely used conventional in vitro method to model absorption of a drug through the intestinal lining uses confluent layers of Caco-2 cells. (See, e.g., Bhatia et al.). This system has several advantages in that it models the absorption of many compounds in the body and it is not as costly as animal studies. However, Caco-2 monolayers have several shortcomings that prevent their use early in the drug screening process. For example, the large footprint of a Caco-2 system (typically a 24-well plate, with approximately 1.2 cm diameter) entails a throughput and expense that are not compatible with screening tens of thousands of candidate compounds. Furthermore, the cells of the monolayer require approximately 3 weeks of constant culturing to achieve confluence and differentiation suitable for analysis (a costly and time-consuming process). In some cases the Caco-2 monolayers do not form properly and drugs can pass through gaps where cells fail to contact and thereby provide false absorption data. It has also been shown that conventional absorbance assays are poor predictors of oral availability for many compounds. (Stewart, B., et al. “Comparison of Intestinal Permeabilities Determined in Multiple in vitro and in situ Models: Relationship to Absorption in Humans.” Pharmaceutical Research, 1995 May; 12(5):693-9; Artursson, P. et al. “Selective Paracellular Permeability in Two Models of Intestinal Absorption: Cultured Monolayers of Human Epithelial Cells and Rat Intestinal Segments.” Pharmaceutical Research. 1993 August; 10(8):1123-9).

The present invention enables the early-stage screening of compound absorption by providing an absorption assay based on the highly precise arraying of single Caco-2 cells. The arraying of single cells eliminates the failure due to incomplete contact between cells and time and expense of fostering a monolayer. This assay is in micro-well array formats that are compatible with conventional methods used for the screening of thousands of compounds, thus enabling the use of this assay in the early stages of drug development.

The area and strength of attachment of the individually arrayed cells can be modulated in the assay system of the present invention to achieve a model that accurately replicates the permeability of the gut. Another feature of this assay system is that the use of single cells allows the assay to model exclusively passive transcellular diffusion (unassisted drug traffic through the junctions between cells and active transport), the most prevalent route of drug absorption. Arrays of single cells are required to obtain sufficient signal-to-noise ratios to detect the absorbed compounds. This system eliminates paracellular transport which can complicate analysis. The elimination of paracellular transport in the provided assay makes it possible to account for transcellular transport more easily than in standard assays.

In the assay system provided by the present invention, provides arraying of single Caco-2 cells, viable cells are arrayed over large areas under culture conditions that allow the differentiation of these cells when cultured in isolation. It is important to note that even in conventional Caco-2 assays, the cells must be highly differentiated and polarized to model absorption properly. The use of microcontact printing optimizes the conditions for single-cell patterning and differentiation of Caco-2 cells. Single-cell patterning is implemented with elastomeric membranes because these structures are ideally suited to the engineering of integrated devices for absorption measurements. In one embodiment of the invention, the device in which the cells are arrayed demonstrates the feasibility of single cell-based absorption and determination of absorption kinetics. In another embodiment, a high throughput version of this assay device is provided.

Currently no methods are available for the arraying of single cells over a large area, e.g., a culture plate. The present invention provides a means to array thousands of single cells using two techniques for the arraying of cells: micro-contact printing and membrane-based patterning. Such techniques may be used in various applications outside the pharmaceutical industry. (See infra Single-cell arraying device).

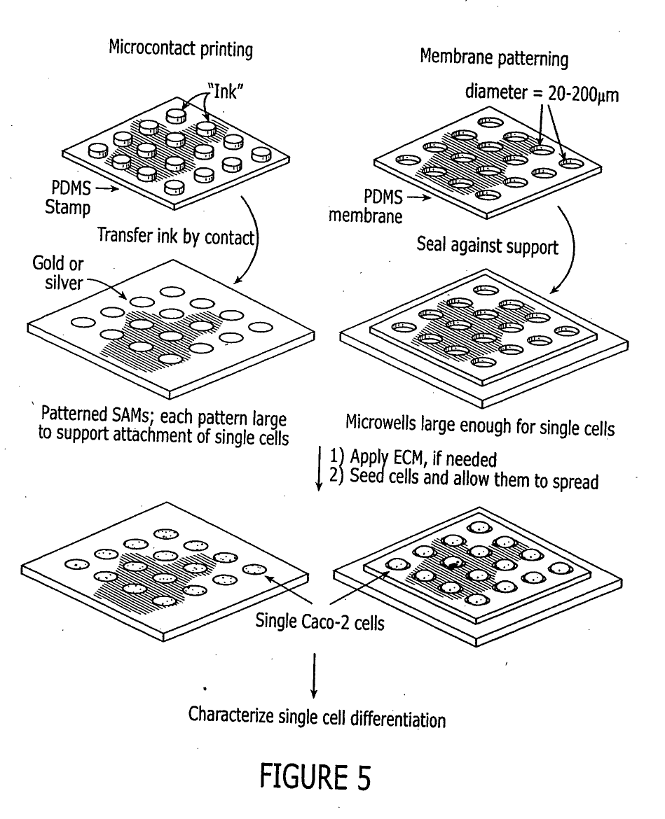

Microcontact printing (mCP) defines arrays for Caco-2 attachment and determines optimal parameters for the differentiation of single Caco-2 cells. Microcontact printing is probably the most versatile and convenient method of patterning biological materials. It uses microfabricated polydimethylsiloxane (PDMS) stamps to print micron-scale patterns of self-assembled monolayers (SAMs); SAMs form well-defined surfaces that can be controlled at the molecular level with a high degree of specificity for biological applications. The present invention uses mCP to create patterns of SAMs that promote adsorption of proteins and subsequent attachment of cells and the surrounding area presents inert SAMs, those that resist protein and cell adhesion (FIG. 5). Optimal culture conditions for single cell differentiation is determined by varying the following parameters: area for cell adhesion, duration of cell culture, ECM proteins (such as fibronectin, laminin, and basement membrane mimics), and density of cells per unit area. All these parameters are easily controlled singularly or in combination using soft lithography and microcontact printing.

After determining optimal culture conditions for Caco-2 differentiation with microcontact printing, a membrane patterning technique is used to place cells in an assayable configuration. This technique uses a thin elastomeric membrane with microfabricated through-holes (20-200 mm in diameter) to define micro-scale culture wells (FIG. 5). The elastomeric membrane is made from PDMS and is sufficiently thick (50-150 μm) to confine cells; the walls of the through holes are arrayed with the components of ECM that have been determined to induce single-cell differentiation and the strongest cell attachment. The top surface of the membrane is modified, if necessary, to resist the attachment and spreading of cells.

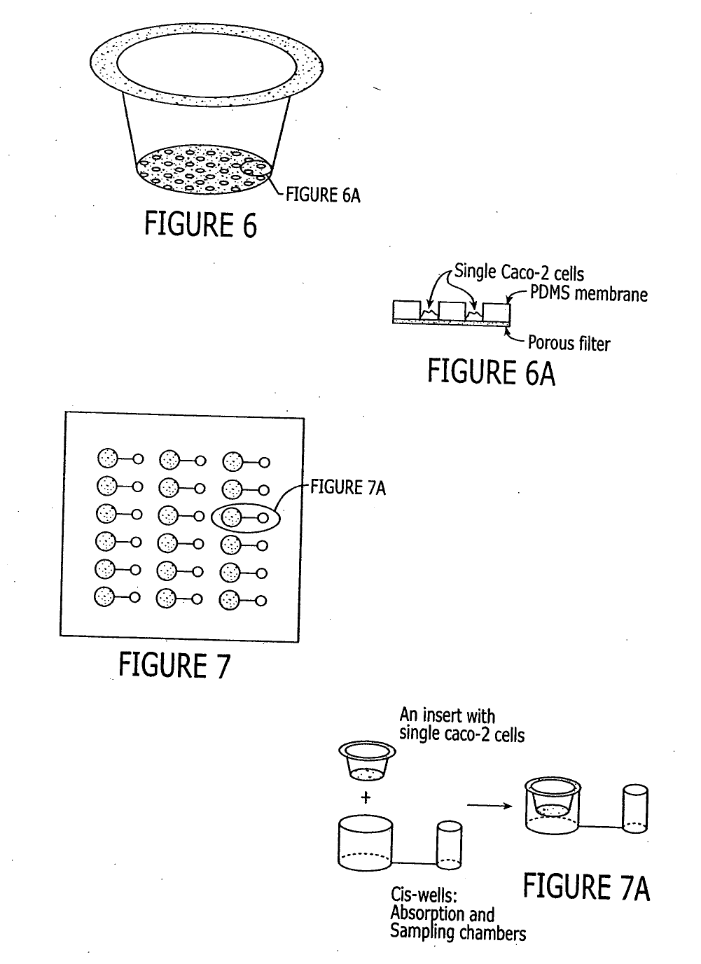

The elastomeric membrane is placed on a thin, porous sheet such as a polycarbonate track etch filter. A number of permeable porous sheets are commercially available and may be tested to identify those with optimal permeability and mechanical properties for device fabrication. Surface chemistry may be used to define the interfacial characteristics of the porous sheets that optimize sealing to the PDMS membrane and interaction with the adsorbed ECM proteins and the basolateral surface of the cells. (Yu, H. and Sinko, P. “Influence of the Microporous Substratum and Hydrodynamics on Resistance to Drug Transport in Cell Culture Systems” Journal of Pharmaceutical Science. 1997; 86, 1448-57). The elastomeric membrane adheres conformally to the porous sheet to create an array of confined attachment sites for cells onto the porous sheet. Since the elastomeric membrane is impermeable to liquid and it forms an impermeable seal with the porous sheet, only drugs absorbed through Caco-2 cells will pass through the porous sheet and will be collected (FIG. 6). The assembly of the porous sheet and the elastomeric membrane is configured as an insert for a well of a 24-well or 96-well plate to allow sampling of the medium above and below the array of CaCo-2 cells using standard equipment.

There are difficulties associated with the arraying and differentiation of single cells. Several labs have tried to array cells in small numbers (about 100) through the deposition of small magnetic elements after decoration of target cells with magnetic material. Alternatively, other groups have used dielectrophoresis to momentarily cage single cells. This system is problematic as it can only be used to capture one cell at a time, and only works on cells in suspension, two qualities that make it unfit for the creation of high-throughput assays or the continued culture of the cells. The present invention's use of microcontact printing or cell-patterning with microstructures fabricated using with elastomeric polymers circumvents the barriers that others have faced. Elastomeric polymers are fabricated more readily into dimensions that would restrict the deposition of cells than are magnetic materials. Furthermore, both microcontact printing and membrane patterning methods, when combined with well-defined surface chemistry, are easily amenable to the arraying of thousands of cells in parallel over the entire assay surface.

The use of PDMS membranes for cell culture provides multiple advantages: 1) PDMS is soft and its softness can be controlled; 2) it is highly permeable to gases; and 3) it is biocompatible and supports long-term culture of cells. The optimal combination of appropriate surface chemistry (i.e., ECM presentation) and physicomechanical characteristics of PDMS provides a culture system that is more “in-vivo-like” than any of the currently available culture methods.

After deposition of the Caco-2 cells, proper differentiation may be characterized by several methods. When Caco-2 cells differentiate in culture, there are distinct biological markers: increased expression levels of brush border hydrolases (e.g. alkaline phosphastase, dipeptidylpeptidase IV, and maltase), carcinoembryonic antigen, and junction proteins. Immunohistology may be used to determine expression levels of these proteins. Phenotypic changes, such as microvilli formation, will be characterized using high-resolution microscopy. Dye permeability across the apical membrane and transepithelial resistance of Caco-2 monolayers may also be measured.

Compositional differences in the polymeric filters on which the Caco-2 cells are arrayed may generate unpredicted interactions with the ECM proteins and the basolateral surface of the cell, thereby affecting the growth and differentiation of the cell monolayer. Furthermore, the permeability of Caco-2 monolayers differs depending on their culture substrate (e.g., polyethelene terephthalate, polycarbonate or aluminum oxide). (Yu, H. and Sinko, P. “Influence of the Microporous Substratum and Hydrodynamics on Resistance to Drug Transport in Cell Culture Systems” Journal of Pharmaceutical Science. 1997; 86, 1448-57). The present invention uses surface chemistry to define the optimal interfacial characteristics to best model the gut lining, thereby overcoming these difficulties.

The predictive ability of the absorbance device may be determined by examining the absorbance of compounds that were well-characterized previously in humans and in Caco-2 assays (Table 1). A tandem HPLC/MS system may be used to characterize absorption. Some of the compounds that may be used to test the system provided herein is listed in Table 1.

TABLE 1
Compounds used to test micro-Caco-2 device

| Compound | Absorbance in vitro | Absorbance in vivo |
|---|---|---|
| Naproxein | High | High |
| Salicylic acid | High | High |
| Chlorothiazide | Low | Low |
| Furosemide | Very Low | Moderate |
| Ephedrine | Moderate | High |
| Nitrozapam | Moderate | Moderate |
| 5-Fluorouracil | Very High | Low |
| Verapamil | High | Very Low |
| Nitrozapam | High | High |
| Mannitol-reference | Low | Very low |
| Propranolol--reference | Moderate | High |

The device of the present invention may be reconfigured into an assay format that can easily be integrated into current drug discovery platforms. The final absorption device outwardly resembles a conventional microtiter plate of 96, 384, or 1536 wells (FIG. 7). Each assay chamber in the device is composed of two adjacent wells connected by microfluidic channels to make up one assay chamber. Absorption occurs in one well containing the insert with a porous sheet described earlier, and characterization or sampling occurs in the other. Each absorption-well contains hundreds of individual Caco-2 cells on a porous membrane, and each sampling well can be interfaced with a standard analytical instrumentation, e.g., sampling robot or a standard plate reader. The assay chamber may be designed to limit evaporation from low-volume microwells. The fluidically connected well configuration offers significant improvements over existing Caco-2 assays in that fresh growth media can be replenished easily from either or both apical and basal sides and density of these absorption assays is much higher than conventional assays (FIG. 7). The low volume, short culture time, and small quantities of reagents required by these assays will reduce their cost. All these characteristics enable high throughput determination of absorptive properties of lead compounds in the early stage of drug discovery process.

Several ways of assaying the absorption of compounds through individual cells patterned on porous supports may be used. The integration of the assay devices provided with standard plate handling, liquid dispensing, and sampling robots is relatively straightforward. Standard detection instrumentation may be configured to interface with the design of the device of the present invention. The use of standard equipment facilitates the integration of the assay provided herein into the current drug development pathway. These systems are amenable to the automated analysis of micro-molar quantities of compound present in microliters of analyte (picomoles of compounds). The system provided by the present invention may be integrated with scanning confocal microscopy to detect morphological changes, spectrometry (tandem HPLC and mass spectroscopy or GC and mass spectroscopy) and spectroscopy (based on absorbance, fluorescence or luminescence) for simple biochemical assays. These assays may also be refitted for time-dependent, high-throughput studies of drug absorption.

The strongest validation of the assay system of the present invention is the accurate prediction of the oral availability in humans of compounds that previous in vitro assays failed to characterize properly or did so poorly. Several compounds have a much higher or lower absorption in vitro than in vivo (see Table 1, above) and these compounds may be used to determine the greater in vivo fidelity of the assay system provided than that of conventional in vitro assays.

An alternative to the use of the single Caco-2 cell assay system is also provided by the present invention: islands that contain a small number of Caco-2 cells (approximately 3-5 cells). These islands also provide better models for absorption than currently existing assays because of the high density, relatively-low time of assay preparation and improved control over cell attachment area and strength of adhesion.

A second alternative assay system is further provided by the use of co-culture of Caco-2 cells with goblet cells using the same methods developed to co-culture hepatocytes. (See infra). The intestinal lining is primarily composed of enterocytes (from which Caco-2 cells are derived) and goblet cells. Goblet cells are the primary producer of intestinal mucus. Incorporation of the mucus layer into an absorption assay may simulate the intestinal environment more accurately.

To achieve better contact of the arrayed cells and their surroundings, the arrayed cells may be transfected with plasmids that enable the over-expression of extracellular proteins that bind to the ECM protein, e.g., cadherins, catenins, integrins and mucins, that is immobilized on the surface of the PDMS membrane. Several pairs of ECM molecules and cell-surface proteins may be tested to find one that sufficiently enhances cell adhesion. Transfection of the DNA that allows expression of cell-surface proteins is accomplished through conventional methods before arraying of the cells or through in-situ transfection, in which arrayed cells take up DNA that is mixed with the ECM molecules on the array surface. In situ transfection by the arraying of hundreds of different plasmids also enables rapid screening of the optimum pairs of ECM molecules and cellular receptors, albeit in a semi-quantitative fashion due to the intrinsic limitations of this technique. In addition to the modification of surfaces with ECM components, chemicals that promote differentiation, such as butyrate, may be added. (Wachtershauser, A. and Stein, J. “Butyrate-induced Differentiation of Caco-2 Cells Occurs Independently from p27.” Biochemical and Biophysical Research Communications. 2001; 281 (2):295-299).

Conventional Caco-2 assays are poor predictors for oral availability because the Caco-2 cells overexpress P-glycoprotein as an artifact of culturing. P-glycoprotein is a major transporter of drugs out of the cell and leads to a depression of absorption rates. The present assay system reduces the expression P-glycoprotein by providing a more complex culture environment such as co-culture, and, alternatively, by molecular biological techniques (such as introduction of anti-sense RNAs or targeted mutagenesis) to reduce P-glycoprotein expression. The levels of P-glycoprotein activity may be studied by following the transport of fluorescently labeled analogs of substrates of the transporter, such as rhodamine-123.

Evaporation, sampling and handling of small volumes of media represent a challenge shared by all high-density cell-based assays. The present assay system has multiple layers and features fabricated at different length scales. Integration is made more challenging by the need for fluid tight seals between layers and sampling within these layers. Such problems may be conquered by a combination of the use of soft lithography and rapid prototyping techniques.

Following absorption, a drug must resist biotransformations that occur primarily in the liver. Biotransformation typically involves three phases (I-III) during which different enzyme families modify the drugs to render them more hydrophilic in order to: 1) inactivate them; 2) reduce the body's exposure to the drug; 3) improve the clearance of the compound to avoid toxic build-up; 4) minimize the toxicity of the compound. The metabolites from each phase of metabolism are the substrates for the subsequent phases. Oxidation, reduction, and hydrolysis occur during phase I, while in phase II the metabolites of phase I are coupled to amino acids, inorganic sulfates, and glucuronic acid or glutathione. During phase III, a combination of the enzymes from phases I and II are active.

Cytochrome P450 enzymes (CYP 450) are the most active class of enzymes during phase I metabolism. The CYP 450 family of enzymes is one of the largest and most important among metabolic enzymes, and its substrates represent the broadest class of compounds than any other system. For these reasons, the pharmaceutical industry has focused most of its pre-clinical metabolic studies on the effect of CYP-450's on drug candidates; such tests, however, are most meaningful in cell-based assays where the effect of the metabolites on cell viability may also be monitored. Another important aspect of pre-clinical testing requires understanding a compound's induction of the expression of CYP 450's which may in turn alter the metabolic properties of the cells and the compound's pharmacokinetics and pharmacodynamics, as well as the body's reaction to other drugs.

To model this metabolic processing, others have attempted to use cultured hepatocytes. The primary goal of these studies has been to understand the effect of the metabolic machinery on the compounds, as well as the compound's effect on the enzyme levels of the cells. In cultured hepatocytes, the physiological levels of the major metabolic enzymes decrease over the course of two weeks. Therefore, assay methods based on conventional hepatocyte culture are unsuited for long-term (>1 month), in vitro studies. Current methods can only be used in short-term, high-dosage studies that are a poor model for the body's low-level, long-term exposure to a drug regimen.

Others have tried to replicate the actions of the liver by hepatocyte co-culture, but fell far short in that the processes used were not of sufficient throughput for pharmaceutical assays or military applications. Previous efforts to use co-culture made no attempt to systematically test the effects of matrix proteins on the stability of hepatocytes. (Bhatia, S. et al. FASEB J. 1999; 13(14):1883-1900, and Bhatia, S. et al. “Probing Heterotypic Cell Interactions: Hepatocyte Function in Microfabricated Co-cultures” Journal of Biomaterials Science-Polymer Edition, 1998; 9(11):1137-1160). Other attempts to model the architecture of the liver failed to demonstrate hepatocyte survival beyond two days. (Kim, S. et al. “Survival and Function of Hepatocytes in a Novel Three-dimensional Synthetic Biodegradable Polymer Scaffold with an Intrinsic Network of Channels” Annals of Surgery. 1998; 228(1):1137-1160).

Primary hepatocytes are fully functional for less than two weeks under current culturing conditions. Over this two week time-span, physiological levels of the major metabolic enzymes decrease and the cells begin to switch to anerobic metabolic states. Therefore, the methods based on conventional hepatocyte culture are unsuitable for long-term in vitro studies as these two factors limit the predictive capability. Current methods can only be used in short-term high-dosage studies that are a poor model for the body's low-level, long-term exposure to a drug regimen. Alternatively, metabolic enzymes can be isolated from microsomes and studied in isolation. However, these procedures are several steps removed from living cells and miss the complex interplay of the various metabolic pathways that transform compounds in the body.

The supply of human hepatocytes is heterogenous, and therefore the expression profiles of metabolic enzymes vary among different samples. This inconsistency in enzyme expression further complicates the ability to compare data obtained with different lots of cells, although it may be important to segment patient populations. The use of standard test compounds for every CYP450 enzyme for calibration during each screening routine is impractical (due to the presence of over 30 such enzymes), time consuming and costly. Methods that use recombinant expression of the enzymes are also limited in scope, because it is believed that only half of the members of this enzyme family have been discovered, based on evidence provided by the human genome project.

In vivo, liver function is carried out in a complex multicellular structure, called the liver sinusoid, which presents a fair degree of order and architecture. In the sinusoid, differentiated hepatocytes surround endothelial structures; in turn, the sinusoid is surrounded by lypocytes and biliary ductal cells that can modify the surroundings of hepatocytes to modulate their function. This structure presents several heterotypic cellular interfaces that stimulate and maintain the hepatocyte phenotype.

The assay device of the present invention uses membrane patterning to establish co-cultures with highly controlled cellular interfaces. The assay device of the present invention also provides a metabolism system based on hepatocytes maintained in culture alongside supporting fibroblast cells. One embodiment of the invention uses co-culture of hepatocytes with fibroblasts because of several factors: 1) they are similar to the supporting cells of the sinusoid; 2) they are relatively easy to maintain in culture; and 3) they are easily engineered to express chosen receptors. Hepatocytes cultured in this fashion retain their phenotypes, functionality, and enzyme expression profile for several months only when the extent of heterotypic cellular interaction is controlled tightly (Bhatia, S. et al. “Effect of Cell-cell Interactions in Preservation of Cellular Phenotype: Cocultivation of Hepatocytes and Nonparenchymal Cells” FASEB Journal. 1999; 13 (14):1883-1900). Additionally, arrayed hepatocytes may be transfected in situ to study the effects of individual cytochrome P-450s (CYPs—the major metabolic enzymes in the liver) on the metabolism of compounds.

Rat hepatocytes may be used to validate the approach of the system provided because of reports of preliminary successes with their stabilization by co-culture with fibroblasts. The assays and device of the present invention may also be used with human hepatocytes to facilitate drug discovery and their use in detectors for anti-human biological agents. The stabilization of hepatocytes allows the establishing of cultures that may represent different patient populations and, therefore, better inform pre-clinical development and patient segmentation for clinical trials. It has often been observed that the variation in the genomic profiles of different patient populations has drastic effects on the therapeutic value of several compounds. The inability to understand these effects has slowed down and in some cases halted the development of drugs causing pain to the untreated patients and financial loss to the pharmaceutical industry.

The assay system provided by the present invention enables the more biologically relevant studies of metabolism, induction, and hepatotoxicity during long-term exposure to low doses of compounds. The lack of hepatocytes that are stable over long periods of time has not made it possible to carry out such studies; to date, the industry has focused on short-term assays at high doses of compound. Certain compounds, however, are known to cause CYP induction and liver toxicity only after long-term exposure (FDA Press Release. “Rezulin to be Withdrawn from the Market” Mar. 21, 2001). Long-term hepatotoxicity and metabolism assays are also required to define the toxic effects of the metabolites of compounds which have a non-toxic original state, as is the case with, e.g., Eulexin (flutamide) and Duract (bromfenac), since it takes time for the metabolites to reach threshold toxic concentrations. Furthermore, the ability to culture cells in a biologically accurate manner for long periods of time enables the integration of living cells into fieldable biosensors (see Hepatocyte-based biosensors, below).

Rat hepatocytes are obtainable through commercial vendors or by standard isolation protocols. Rat hepatocytes are cultured alongside 3T3-J2 fibroblasts from the ATCC. Human hepatocytes are isolated according to published protocols. (For example, Seglen, P. “Isolation of Hepatocytes” In Cell Biology: a Laboratory Handbook, 2^{nd} Ed. Volume 1, Celis, J ed. Academic Press, San Diego, 1998 and Models for Assessing Drug Absorption and Metabolism, Borchardt, R et al. Eds. Pharmaceutical Biotechnology Series, Vol. 8.).

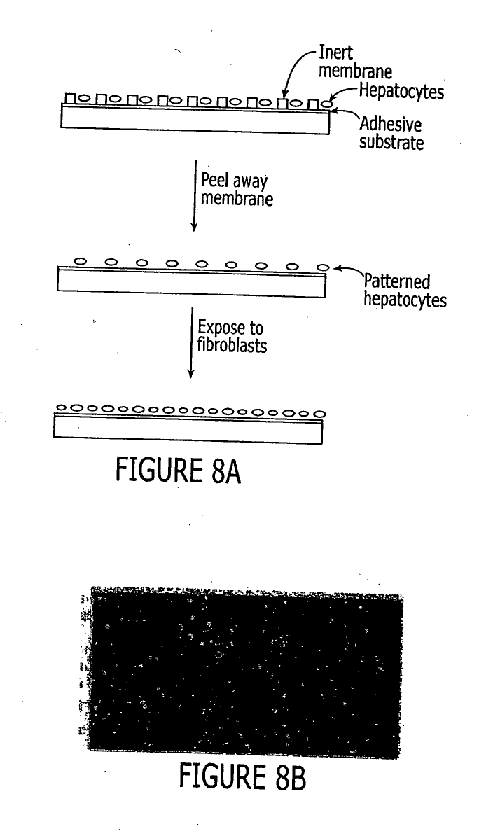

Several different matrix proteins may be used for their ability to optimize hepatocyte attachment and survival. Candidates of matrix proteins to enhance the attachment of hepatocytes include, but are not limited to, collagen, fibronectin, laminin and vitronectin. Once the support of the device is properly modified with matrix proteins, an elastomeric mask is placed over the support to restrict the area to which hepatocytes can attach. After hepatocyte attachment, the mask is removed and fibroblasts are allowed to attach to the newly revealed area surrounding the hepatocytes (FIG. 8). The geometry and area of the adhesion space is configured to optimize heterotypic cell-cell interactions on the surface. (Bhatia, S. et al. “Probing Heterotypic Cell Interactions: Hepatocyte Function in Microfabricated Co-cultures” Journal of Biomaterials Science-Polymer Edition, 1998; 9(11):1137-1160).

This technique may be adapted to pattern a different protein for each cell type. Surface chemistry may be applied to the oriented, biospecific, homogenous immobilization of proteins to the assay surfaces. The levels of CYP enzyme expression may be tested further with well-characterized compounds (Table 2).

The stability and performance of the co-cultured hepatocytes is assessed by comparing their urea synthesis, albumin secretion, CYP enzyme production and oxygen metabolism against physiological levels. Furthermore, hepatocytes may also be tested for their ability to metabolize well-characterized compounds (Table 2).

TABLE 2
Selected CYP450 enzymes, their substrates and products that modify
their expression (Reproduced from: Ioannides, C. Cytochrome P450:
Metabolic and Toxicological Aspects. CRC Press, New York, 1996 p. 33).

| | | Products that modify P450's |
|---|---|---|
| P450 Isozyme | Substrates | expression |
| CYP1A2 | Phenacetin, caffeine, | Omperazole |
| | aflatoxin B | |
| CYP2A6 | Coumarin, | Pyrazole |
| | dimethylnitrosamine | |
| CYP2C8 | Tolbutamide, S-warfarin | Rifampicin |
| CYP2E1 | Ethanol, carbon | Ethanol, isoniazid |
| | tetrachloride | |
| CYP3A4 | Cyclosporin, Nifedipine | Dexamethasone, rifampicin |

The format of the device of the present invention is based on a 96-well culture plate. Each well in the plate contains approximately 100 islands of approximately 1-5 hepatocytes surrounded by fibroblasts. Metabolized compounds are collected from above the layer of cultured cells using standard liquid handling equipment.

The metabolized compounds may be analyzed with simple detection techniques such as fluorescence, HPLC and GC. Moreover, all standard assay techniques are suitable for interface with the assay system provided herein, e.g., tandem mass spectrometry-HPLC. These detection systems have sufficient sensitivity to detect the metabolic activity generated by ensembles of 100-500 hepatocytes.

The density of the metabolism assay must be increased to make it suitable for integration into the early stages of drug development, which requires the integration of a removable patterning microstructure from each well in a high-density plate.

Stabilized hepatocytes are used to trace the long-term induction of CYP enzymes caused by exposure to test compounds. Side effects and dosing problems observed in some compounds are attributable to up- or down-regulation of CYP enzymes caused by drug exposure. (For example, see, Henney, J. “Risk of Drug Interactions with Saint John's Wort” JAMA4, 2000; 283(13)). Induction of CYPs by a compound can be problematic because it alters the pharmacokinetics of other therapeutic agents to which a patient may be exposed. This induction can be detected using standard molecular biology techniques (such as DNA array or Western blot) or by comparing activity levels of harvested CYPs. Induction of CYPs is often a more sensitive indicator of compounds unsuitable for therapeutic use than any gained from the analysis of a compound's metabolites.

The ability to stabilize hepatocytes over months allows for the configuration of assays for the determination of long-term hepatotoxicity of drugs and environmental agents. Therefore, in another embodiment of the present invention, these hepatocytes are used in an assay to determine the long-term hepatotoxic effects of drugs and environmental agents, thereby reaching another parameter desired in ADMET testing. To determine if a compound has delayed liver toxicity, hepatocytes are maintained in culture in the presence of low-doses of suspect agents. This assay is similar in format to the herein described metabolism assay except for the fact that detection is based on the release of alpha glutathione S-transferase or alanine transaminase (enzymes stored in high concentrations in hepatocytes and released upon cell death) into the media instead of on an analysis of the compound metabolites. Long-term toxicity may be tested in the assay device using known compounds such as flutamide or bromfenac.

The high-throughput and long-term capacity of the presently provided assay system enables the study of drug-drug interactions. As the knowledge of medicine increases, more people take or will have to take several medications daily (e.g., cholesterol-lowering drugs with allergy medication or anti-depressants). One drug may drastically alter the metabolism of another drug, thereby affecting the therapeutic outcome. Drug-drug interactions can be assessed by the direct analysis of altered metabolites, CYP induction assay or by hepatoxicity assay. The microtiter plate format is extremely useful for the testing of several drugs simultaneously as each compound can have its own row or column in the plate, making a matrix of interacting compounds. Conveniently, assays of the present invention for metabolism, hepatotoxicty, CYP induction and the drug-drug interactions are facilitated by common device, protocols and detection methods.

The presently provided long-term hepatotoxicity and induction assays are useful to test the effect of trace compounds found in food that may be dangerous to consumers. Currently the USDA uses animal models to determine the long-term toxic effects of food additives. The presently provided assay will advantageously provide a quicker and less expensive screen for compounds prior to animal testing.

The key to hepatocyte stabilization is their placement in a culture system that surrounds them with the proper density of fibroblasts to maximize the heterotypic cell-cell interactions necessary to maintain the functional state of the cells. The assay device of the present invention uses soft lithography to define areas for the attachment of hepatocytes and supporting cells. Photolithography, which is sometimes described as an alternative to soft lithography, is difficult to implement and is incompatible with the deposition of the biomolecules that mediate adhesion.

To optimize the functional state of the co-cultured hepatocytes in the presently provided in vitro system, the effects of the ECM proteins on surfaces, the ratio of hepatocytes to fibroblasts, and the average number of hepatocytes per microscopic island are investigated and adjusted, as needed. For example, cell types other than fibroblasts that are found in the liver may be used to optimize hepatocyte stabilization; sinusoidal, endothelial, or biliary duct cells are examples of such cells.

There are several marked differences between the metabolism carried out by human and rat hepatocytes, and therefore the most faithful replication of metabolism that occurs in the human body necessitates the use of human hepatocytes. (Ioannides, C. Cytochrome P450: Metabolic and Toxicological Aspects. CRC Press, New York, 1996). The use of human hepatocytes is essential for the development of biosensors that are designed to detect human disease agents (See Further Applications infra). It is hypothesized that the micro-architecture of the liver in both animals is similar enough that established culture techniques used for rat hepatocytes will be applicable to the culture of human cells with reasonably simple changes.

There is a need in the pharmaceutical industry to have one in vitro assay that models the interaction of compounds with the body from their absorption, metabolism and distribution to their final elimination from the body. Also, the ultimate goal of real time prediction of human response to compounds requires the linking of several assay systems into an integrated whole. The presently invention further provides an integrated absorption and metabolism assay and device, i.e., an integrated ADMET assay.

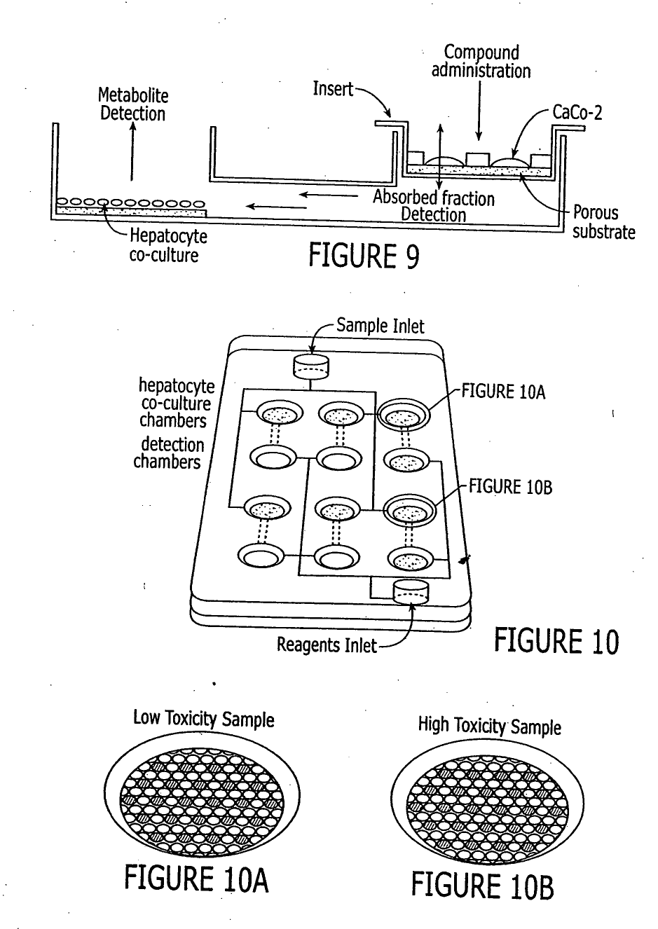

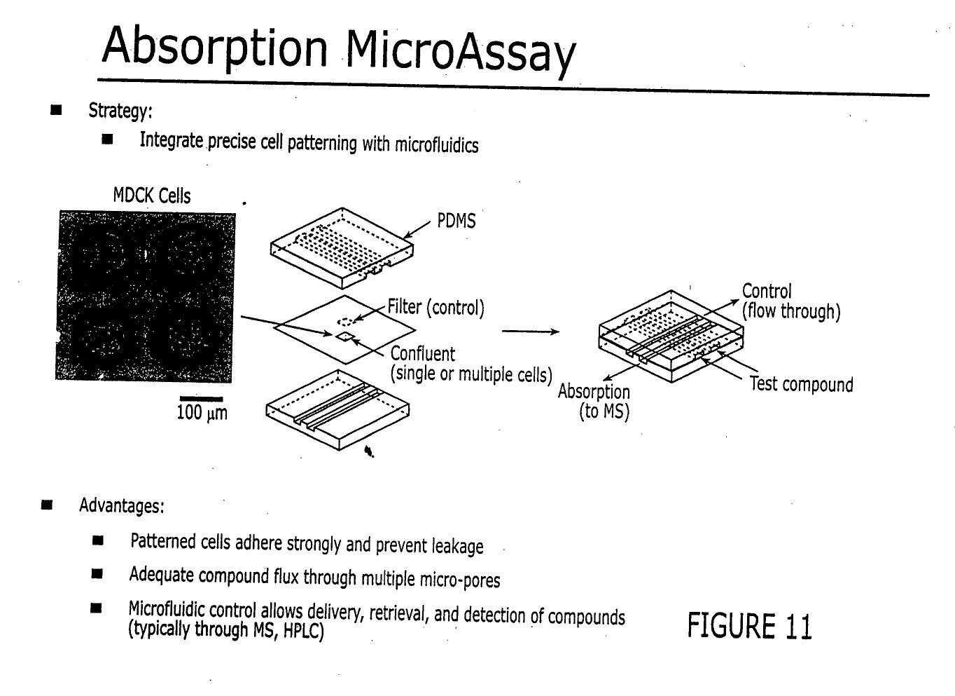

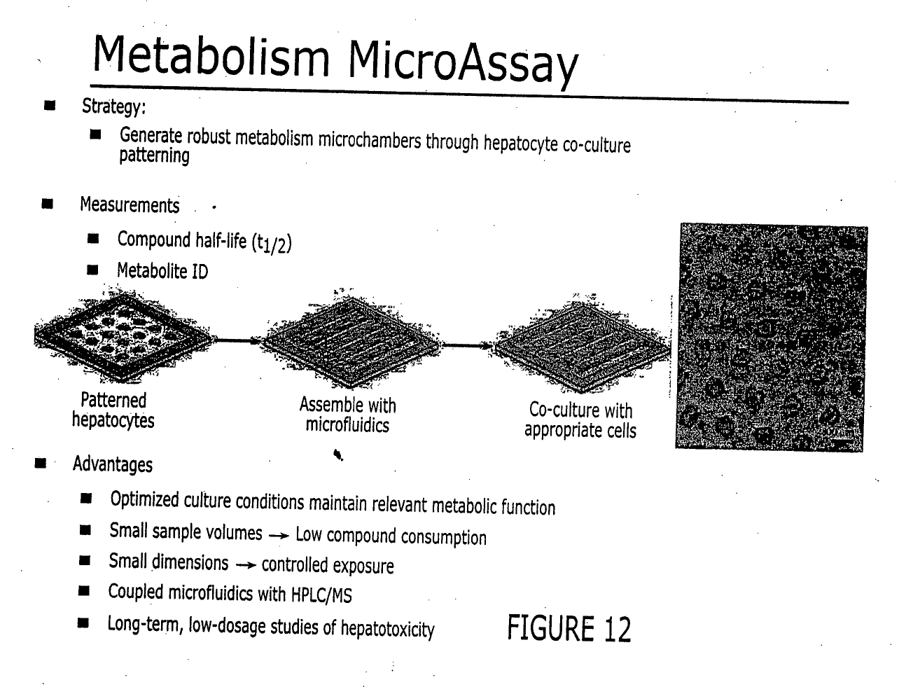

The present invention provides single assay system, by integrating the absorption and metabolism assays also provided herein, that can determine what fraction of a compound gets absorbed, and how the absorbed fraction is metabolized by the body (FIG. 9). The absorption and metabolism assay chambers are modular, enabling separate growth and maintenance of the cells prior to the assay. This modularity also enables the use of any individual assay separately or together. The integrated assay may thus be used for high-throughput drug screens. In an embodiment, a low-throughput, e.g., a 24-well integrated assay device is also provided. Ultimately, this invention provides an assay that may be used in the 96- or 384-well format (or larger), so that it can easily be integrated into current drug discovery protocols with sufficiently high-throughput.

The assay devices of the present invention include diagnostic devices that can analyze medical and environmental samples for the presence of pathogens, toxins and hepatotropic viruses, which will be useful as hepatocyte-based biosensors, e.g., for the defense community. In addition to immediate utility, the assay devices of the present invention further provide a device that can predict the response of a person to toxins and microbes in the field. These devices share a common design derived from the demonstrations carried out for the absorption and metabolism assays described above. The different assay chambers are connected by microfluidic channels that will allow continuous flow of sample as well as its partitioning into multiple assays. (FIG. 10).

Current biological weapon detector technology requires that devices be pre-loaded with reagents or probes, e.g., antibodies or DNA probes, targeted to a specific subset of pathogens. Hostile actors may avoid the use of pathogens on the “agents of concern” list and thereby render their weapons invisible to current detection schemes. Biological weapon detectors based on the reaction of living cells to pathogens are able to sense the presence of any pathogen that alters the behavior of the cell type used in the sensor, regardless of whether that agent is an emerging pathogen, engineered or outside of expected weaponizable agents.

The development of culture methods that support the long-term survival of hepatocytes will enable the development of devices that use these cells as sensors to detect the presence of chemical and/or biological agents. Several systems that use living cells as sensing elements in biological weapon detectors are under development. One such device of this type is the CANARY system, under development in the MIT Lincoln labs, that uses genetically-engineered B-cells to sense the presence of pathogens. The lifespan of this device is limited by B-cell culture stability (approximately 2 weeks). Several labs have made prototype biosensors using fish, rat or duck hepatocytes to detect toxins in samples (For example, see, Grant, G M et al. “JP-8 Jet Fuel-induced DNA Damage in H411E Rat Hepatoma Cells” Mutation Research, 2001; 490(1):67-75). These cells have a limited life-span, and lose their functionality quickly because they are not stabilized in any way. The cell-based biosensors of the present invention may also be applicable to other cell lines, through improvements in the increase of the functional lifespan of the sensing cells.

The present invention provides a cell-based biosensor to detect acutely toxic samples using hepatocytes co-cultured with fibroblasts. As discussed above, the cells in this biosensor possess a long functional life-span due to their co-culture with supporting fibroblasts. Prematurely dying hepatocytes release alanine transaminase or alpha glutathione S-transferase into the media. This release is detected by automated, fluorescent-linked, immunoabsorbant assay. Briefly, samples are delivered to the hepatocytes via microfluidics (FIG. 10), and growth media is carried in microchannels from the culture wells to the test wells where anti-transaminase or transferase antibodies are immobilized. The antibodies may be detectably labeled, e.g., fluorescently labeled, to detect enzyme bound to the immobilized antibodies. For acutely toxic samples, one or a few chambers of hepatocytes are exposed to many samples serially until a sample that contains a toxin is detected. For the evaluation of long-term toxicity, samples are delivered to separate chambers and the survival of hepatocytes in the presence of each of the samples will be monitored over time.

The present invention provides a biosensor that can be used to detect acutely toxic samples such as those encountered during chemical warfare. For example, cellular response may be tested to aflatoxin, a toxic agent which was weaponized and loaded into munitions by Iraq. The toxic effects of aflatoxin on the liver are immediate (it is metabolized into a diol-epoxide that alkylates DNA), yet the damage caused by the toxin leads to cancer many years after exposure instead of immediate death of the victim. Aflatoxin is also within reach of sub-state actors due to the relatively low technical barriers to its acquisition and use and its suitability to sabotage operations.

The chemical biosensor provided by the present invention may be modified for the detection of hepatotropic viruses. The most obvious use of this biosensor is the detection of hepatitis viruses, newly included in the list of “agents of concern” due to their relative ease of acquisition and dissemination. Several viruses that are significant public health concerns can infect the liver, including cytomegalovirus, rubella virus, herpes simplex virus, human herpes virus 6, varicella, coxsackievirus, echovirus, reovirus 3, parvovirus B19, HIV and paramyxovirus. The presently provided system may also be used to detect virus-based agents of concern that attack the liver, e.g., several hemorrhagic fevers (including Lassa, Rift Valley and Ebola), Dengue fever virus, yellow fever virus, sandfly fever virus. Samples are delivered to isolated chambers of co-cultured hepatocytes (FIG. 10). If the sample contains hepatotropic viruses, cell death should be observable within days through cell viability tests or supernatant immunoassays as described above. The assay device may be used to detect cytomegalovirus, varicella virus and Punta Toro virus, which are used as model systems for other hepatotropic, viral, biological warfare agents, e.g., Lassa HFV.

The hepatocytes used in the assays of the present invention may also be engineered to express receptors for the recognition of non-hepatotropic pathogens. Preliminary steps toward engineering cultured hepatocytes for this purpose have been done to eventually allow the detection of lipopolysaccharide, an important bacterial surface antigen. (Vodovotz, Y. et al. “The Hepatocyte as a Microbial Product-responsive Cell” The Journal of Endotoxin Research, 2001; 7(5):365-73). This work stopped far short of actually enabling hepatocytes to sense the presence of bacteria, but it is an important first step. Clearly, this engineering work is of limited utility if the hepatocytes used to sense the pathogens have a short functional life in the device.

Although not required for the successful implementation of the absorbance assay provided by the present invention, the assay may be modified to integrate a goblet cell/Caco-2 cells co-culture into the absorbance assay to model more accurately the intestine. This assay integrates the techniques of both the absorbance and metabolism assays provided herein.

The ultimate goal of any pharmaceutical assay system or biological sensor is to predict the physiological responses of a human to the compounds or organisms it may encounter in the environment. The assay devices and methods of the present invention seek to model the body's response to a compound from its initial entry into the body, through its absorption, metabolism and final elimination from the body by connecting the distinct, highly-biologically relevant assays through microfluidics that mimic the vasculature and digestive systems. The devices and assays provided herein will extend to cell types that mimic more accurately all relevant physiological absorption barriers such as transdermal and blood-brain. The high-throughput system provided should therefore be suitable for use in the early stages of drug development, eliminating candidate compounds that have undesirable absorption, metabolic, toxic or elimination properties. Furthermore, this system may be used to characterize the effects of new toxins that a soldier may encounter on the battlefield without having to first characterize the toxin. The provided assay system also forms the basis for future assays in which components of the immune system may be linked to the vascular or digestive system to model the body's interaction with new pathogens. The assays of the present invention allow for the differentiation of an agent based on its preferred route of entry into the body using the advanced absorption assays and based on its toxicity on competent cells. Ultimately, these linked physiological modules will assist in the rapid detection and characterization of emerging threats and engineered biological weapons.

The present invention provides an integrated ADMET assay that models absorption, metabolism, toxicology and elimination of a compound in a high-throughput formation the pharmaceutical industry. This assay device provided also forms the basis of a low-throughput, more robust system that may be fielded to characterize new toxins used by an aggressor.

The arraying of cells individually is a technology essential to the development of the provided absorption assay. In addition to the aforementioned benefits of improving the current absorption assays, the ability to array cells individually has several powerful applications. By arraying thousands of hybridoma cells and interfacing with a platform to analyze the produced antibodies, the assay device enables the rapid production of monoclonal antibodies for therapeutics and diagnostic purposes, e.g., for the treatment of emerging infectious diseases and engineered pathogens.

The ability to array mammalian cells individually has the potential to revolutionize mammalian genetics. Microbes have traditionally been the organisms of choice for molecular genetics due to their ability to grow in colonies. Each colony grows from a single founding cell and is genetically identical to other individuals in the colony. A single plate may have hundreds to thousands of distinct colonies, each of which may have a distinct genetic makeup (such as after mutagenesis or transformation with a genomic library). Through the use of the single cell arrayer (assay device) provided herein, mammalian cells may be cultured in a manner similar to microbes: growth in thousands of genetically diverse but isolated cell populations. Each population will have been founded by a single cell, maintaining genetic homogeneity within the population. This culture system thereby enables the cloning of genes responsible for observed phenotypes in mammalian cells through the use of standard techniques of molecular biology. Phenotypic cloning will increase the speed at which genes can be linked to new genetic diseases and allow fingerprinting of the mutations of cancer cells from a patient to determine the most effective chemotherapy.

In another aspect of the present invention, provided are culture systems with integrated fluid sample delivery mechanisms to mimic the in vivo physiology of various organs and/or of the body, including the liver. Such a co-culture system has applications in various fields including analysis of test compound metabolism, measurement of compound hepato-toxicity, and reaction of patient disease states to treatment. Several cell patterning methods and device configurations may be used to co-culture cells in the ADME/Tox systems provided herein. The preferred coculture systems of the present invention use primary hepatocytes combined with cells of a fibroblast cell line, but many other cell types may be patterned together for coculture using this technology, as described below. ‘Primary’ cells, as used herein are defined as cells freshly acquired and isolated from a live patient or animal. Frozen and thawed hepatocyte cell populations have been shown to display about a two-fold decreased biochemical activity, as well as altered cell viability and altered cell morphology, when compared to freshly isolated, i.e., primary, cell populations. (See, Alexandre, E. et al. Cryobiology, 44: 103113 (2002), the entire contents of which are incorporated by reference in their entirety herein, specifically methods of isolating and culturing primary cells and assays for biochemical activity.

In one example embodiment, this invention provides a device for co-culturing at least two different cell types in a two-dimensional configuration comprising a cell culture support surface; and a microfluidic system having a removable patterning membrane disposed on the cell culture support surface and a plurality of channels for flowing cells to surfaces exposed within the channels, wherein the channels are in conformal contact with the cell culture support surface and are parallel relative to each other and spaced apart relative to each other.

In one example embodiment, this invention provides a device for co-culturing at least two different cell types in a two-dimensional configuration comprising a cell culture support; and at least one removable membrane disposed on the cell culture support, wherein the membrane forms a stencil pattern on the cell culture support.

In another example embodiment, this invention provides a method of patterning at least two different cell types in a two-dimensional co-culture configuration comprising: a) providing a device having a cell culture support surface; and a microfluidic system having a removable patterning membrane disposed on the cell culture support surface and a plurality of channels for flowing cells to surfaces exposed within the channels, wherein the channels are in conformal contact with the cell culture support surface and are parallel relative to each other and spaced apart relative to each other; b) flowing cells of one tissue type through one set of alternating channels to form multiple rows of contiguous cells of a first tissue type within the channels, wherein the rows are parallel relative to each other and spaced apart relative to each other; c) removing the removable microfluidic patterning membrane from the cell culture support to form alternating rows of bare cell culture support contiguous with and parallel relative to the rows of contiguous cells of step (b); and d) flowing cells of a second tissue type through a second set of alternating channels to the alternating rows of bare cell culture support of step (c), to form rows of contiguous cells of the second tissue type contiguous with the rows of contiguous cells of the first tissue type on the cell culture support.

In another example embodiment, this invention provides a method of patterning at least two different cell types in a two-dimensional co-culture configuration comprising: a) providing a device having a cell culture support; and at least one removable membrane disposed on the cell culture support, wherein the membrane forms a stencil pattern on the cell culture support; b) applying cells of one tissue type to open areas formed by the stencil pattern, wherein the open areas are spaced apart relative to each other; c) removing the at least one removable membrane from the cell culture support to form bare areas of cell culture support; and d) applying cells of a second tissue type to the bare areas cell culture support.

In a further example embodiment, this invention provides a method of patterning at least two different cell types in a two-dimensional co-culture configuration comprising: a) providing a non-coated cell growth substrate, wherein the substrate has a plurality of patterned electrodes embedded within said substrate and a plurality of electroactive cytophobic self-assembled monolayers (SAMs) patterned onto the cell substrate; b) applying cells of a first tissue type to the non-SAM coated cell growth substrate; c) desorbing the plurality of electroactive cytophobic SAMs from the cell substrate to form cell adhesive regions in the pattern of the removed SAMs; d) activating at least one electrode to form at least one activated region of the cell growth substrate; e) applying cells of a second cell type to the at least one activated region of step (d) to form a pattern the cells of the second cell type in at least one activated region, thereby patterning at least two different cell types in a two-dimensional co-culture configuration.

In further embodiments of the above-described devices, wherein the channels have a diameter of 10 to 500 microns. The smallest feasible size for one cell is 10 microns, but channels as large as 200 micron diameter or larger are useful in the devices of the present invention for cells having larger feature sizes. For example, for hepatocyte coculture, 50, 200 and 500 micron diameter channels may be used, while for fibroblasts channels of at least 20 microns up to 500 microns are used. For endothelial coculture, channel diameters of 25 to 50 microns are optimal for capillary formation (e.g., for studies of angiogenesis), but larger diameter channels may also be used.

In additional embodiments of the above-described devices, the removable patterning membrane is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

In further embodiments of the devices provided herein, the device may further comprise a plurality of overlapping removable membranes or a plurality of nonoverlapping removable membranes.

In further embodiments of the patterning and coculture methods provided herein, the methods may further comprise culturing the cells of the first tissue type with the cells of the second tissue type in the two-dimensional co-culture configuration. In other embodiments, the methods may further comprise contacting the rows of contiguous cells of the first tissue type or the second tissue type with a drug before culturing. The methods may also further comprise contacting the rows of contiguous cells of the first tissue type with a first drug before step (e) and contacting the rows of contiguous cells of the second tissue type with a second drug before culturing.

In all of the patterning and coculture methods provided herein, the cells of the first tissue type may be primary cells (freshly isolated), cultured cells, thawed cells, wherein said cells have been isolated and frozen prior to thawing, or immortalized cells. Likewise, the cells of the second tissue type may be primary cells (freshly isolated), cultured cells, thawed cells, wherein said cells have been isolated and frozen prior to thawing, or immortalized cells in all methods provided herein.

The method may further comprise culturing the cells in a two-dimensional co-culture configuration. As used herein, “two-dimensional configuration” (“2-D”) is defined as cell to cell contact in a plane (on a planar surface), wherein the cells are contiguous with each other, i.e., in contact with each other, preferably in continuous contact, i.e., unbroken by bare space. Such contact is not contiguous on all sides of the cells, i.e., the cells are not completely covered by each other (in homotypic cultures) or a second cell type (in heterotypic cell cultures) in three-dimension. Fro example, cells in coculture in a 2-D configuration are disposed on a flat surface or porous membrane not in contact with other cells on the top or bottom thereof, all lateral sides thereof are in contiguous contact with other cells of the culture. (However, in additional embodiments described below, a third cell type may be added to overlap cells already in a 2-D configuration and such coculture is in fact a 3-dimensional coculture.) As described herein, optimal cell to cell contact of about 35% permits longest cell viability with function and morphology of the cells maintained closest to that of said cells in vivo.

The methods provided herein may further comprise using a device having a plurality of overlapping removable membranes. Such methods may further comprise i) removing one overlapping removable membrane; and ii) applying cells of a third tissue type to the overlapping areas, wherein said areas overlap either the cells of the first tissue type or cells of the second tissue type. These method may further comprise i) removing one overlapping removable membrane; and ii) contacting the overlapping areas with at least one drug, wherein said areas overlap cells of either the first tissue type or the second tissue type. The methods described may further comprise using a device having a plurality of nonoverlapping removable membranes.

These methods may further comprise: i) removing at least one nonoverlapping removable membrane to form bare areas of cell culture support, wherein said areas are contiguous with either the cells of the first tissue type or cells of the second tissue type; and ii) applying cells of a third tissue type to the bare areas. These methods may also further comprise: i) removing one nonoverlapping removable membrane to form bare areas of cell culture support, wherein said areas are contiguous with either the cells of the first tissue type or cells of the second tissue type; and ii) contacting the bare areas with at least one drug.

In a further example embodiment, this invention provides a method of patterning at least two different cell types in a two-dimensional co-culture configuration comprising: a) providing a non-coated cell growth substrate, wherein the substrate has a plurality of patterned electrodes embedded within said substrate and a plurality of electroactive cytophobic self-assembled monolayers (SAMs) patterned onto the cell substrate; b) applying cells of a first tissue type to the non-SAM coated cell growth substrate; c) desorbing the plurality of electroactive cytophobic SAMs from the cell substrate to form cell adhesive regions in the pattern of the removed SAMs; d) activating at least one electrode to form at least one activated region of the cell growth substrate; e) applying cells of a second cell type to the at least one activated region of step (d) to form a pattern the cells of the second cell type in at least one activated region, thereby patterning at least two different cell types in a two-dimensional co-culture configuration.

In additional embodiments of the above-described method, such method may further comprise: i) sequentially activating at least one second electrode to form a second activated region of the cell growth substrate; ii) applying cells of a third cell type to the at least one second activated region of step (d) to form a pattern of the cells of the third cell type in at least one second activated region, thereby patterning at least three different cell types in a two-dimensional co-culture configuration. In other embodiments of the above-described method, such method may further comprise: i) activating a plurality of electrodes in step (d) to form an activated pattern on the cell growth substrate; ii) applying cells of a third cell type to the activated pattern to form a pattern of cells of the third cell type in the activated pattern, thereby patterning at least three different cell types in a two-dimensional co-culture configuration. The above-described methods may further comprising repeating steps (i) and (ii) to sequentially apply an additional different cell type and form a pattern therewith. In all of these methods the patterned electrodes may form regions of round islands, wherein said islands are spaced apart relative to each other. Alternatively, the patterned electrodes may form regions of elongated strips, wherein said strips are parallel relative to each other and are spaced apart relative to each other. Each elongated strip is at least 20 microns wide to form patterns of strips of single cells. Each elongated strip may be from at least from 100 microns wide to 500 microns wide to form patterns of strips of multiple cells within said strips. The cells of the first tissue type or second tissue type may be primary cells, cultured cells, thawed cells, wherein said cells have been isolated and frozen prior to thawing, or immortalized cells. In an example embodiment, cells of the first tissue type may be hepatocytes and cells of the second tissue type may be fibroblasts. In further embodiments, cells of the third tissue type may be added, for example endothelial cells. In other embodiments the cells of the first tissue type and the second tissue type are from the same subject. Preferably, the subject is a mammal, most preferably the mammal is a human. The above-described methods may further comprise contacting the cells of the first tissue type with a therapeutically effective amount of at least one drug. Additionally, the methods may further comprising contacting the cells of the second tissue type with a therapeutically effective amount of at least one drug. In all of these methods, the cells of the first tissue type may be from a first subject and the cells of second tissue type may be from a second subject, said second subject being different than the first subject. For example, the cells of the first tissue type are from a first mammal and the cells of second tissue type are from a second mammal, said mammal being from a different species. The first mammal may be a human and the second mammal may be a mouse, rat or pig.

In other embodiments of the above-described methods the cells of the first tissue type are diseased cells from a subject and the cells of second tissue type are from said subject, wherein the cells of the second tissue type are located proximate to the cells of the first tissue type in the subject. For example, the cells of the first tissue type may be hepatocytes, said hepatocytes being cancerous, cirrhotic of infected and cells of the second tissue type may be fibroblast, preferably, or endothelial cells.

In one example embodiment, this invention provides a device comprising: at least three layers, said layers being a first layer, a top layer and a middle layer, wherein the first layer is a lower layer having fluid inlet receptacles and fluid outlet receptacles, said receptacles being connected by a microfluidic system, wherein the top layer has a cell culture well and an opening to said fluid inlet receptacle and fluid outlet receptacles and wherein the middle layer is configured to receive cells on its top surface, said layer being porous and separating the cell culture well from the microfluidic system. Such a device is known herein as an extravasation device or a transmigration device or a transmigration and extravasation device.

In further embodiments of the above-described device, cells are patterned on top of the middle layer in a two-dimensional co-culture configuration. The pattern of the two-dimensional co-culture configuration may be a round island pattern or an elongated strip pattern.

In another example embodiment, this invention provides a device comprising: a housing defining at least one chamber therein; a membrane disposed in the at least one chamber and defining a plurality of micro-orifices, the membrane being configured such that each of the plurality of micro-orifices is adapted to receive a single cell therein, and such that the at least one chamber includes a first region on one side of the membrane, and a second region on another side of the membrane; a delivery device in fluid communication with the first region of the at least one chamber, the delivery device being adapted to deliver a fluid to the first region; and a removal device in fluid communication with the second region of the at least one chamber, the removal device being adapted to remove a fluid from the second region.

In further embodiment of the above-described device, the housing and the membrane are configured such that fluid is adapted to pass from the first region to the second region through the plurality of micro-orifices. In another embodiment of such devices the housing and the membrane are configured such that fluid is adapted to pass from the first region to the second region only through the plurality of micro-orifices. In additional embodiments of these devices, the plurality of micro-orifices are arranged in a predetermined pattern that corresponds to a pitch of a standard microtiter plate. The predetermined pattern corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate. In other embodiments of these devices, each of the plurality of micro-orifices has a diameter from about 10 microns to about 50 microns. The membrane of these devices is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone. In further embodiments of the above-described devices, the at least one chamber comprises a plurality of chambers.

The plurality of chambers may be attached to each other. The plurality of attached chambers may be arranged in a grid or arranged as a strip. The plurality of chambers of these devices define a pitch relative to one another that matches a pitch of a standard microtiter plate. The plurality of chambers define a pitch relative to one another that matches of pitch of a 6-well microtiter plate, a 12-well micerotiter plate, a 24-well microtiter plate, a 96-well microtiter plate, a 384-well microtiter plate, a 1,536-well microtiter plate, and a 9,600-well microtiter plate. In any of the above-described devices, the delivery device may be a microfluidic device, a pipette or a robotic device. In these devices, each of the plurality of micro-orifices define walls, and wherein the device further comprises a surface coating on the walls of at least one of the plurality of micro-orifices. The devices may further comprise a filter layer disposed in the second region of the at least one chamber. The filter layer defines a plurality of micro-pores each having a diameter of about 2 microns to about 5 microns. The filter layer is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone. In further embodiments of the above-described devices, the device has a trans-configuration, the membrane being substantially horizontal in a test orientation of the device or the device has a cis-configuration, the membrane being substantially vertical in a test orientation of the device.

In a further example embodiment, this invention provides a device comprising: a housing defining at least one chamber therein; a plurality of membranes, each of the membranes defining a plurality of micro-orifices and being configured such that each of the plurality of micro-orifices is adapted to receive a single cell therein, the membranes being disposed in the at least one chamber such that the at least one chamber includes a first region on one side of the membranes, and a second region on another side of the membranes; a delivery device in fluid communication with the first region of the at least one chamber, the delivery device being adapted to deliver a fluid to the first region; and a removal device in fluid communication with the second region of the at least one chamber, the removal device being adapted to remove a fluid from the second region.

In an embodiment of the above-described device, the housing and the membranes are configured such that fluid is adapted to pass from the first region to the second region through the plurality of micro-orifices. In a preferred embodiment, he housing and the membrane are configured such that fluid is adapted to pass from the first region to the second region only through the plurality of micro-orifices. (i.e., through a cell disposed on the membrane) In an embodiment of the device, the at least two of the plurality of membranes are substantially parallel relative to each other. In another embodiment of the device, each of the plurality of membranes are substantially parallel relative to each other. In other embodiment of the device, the at least two of the plurality of membranes are spaced apart relative to each other. In a further embodiment of the device, each of the plurality of membranes are spaced apart relative to each other. In additional embodiments, the plurality of micro-orifices of each of the membranes are arranged in a predetermined pattern that corresponds to a pitch of a standard microtiter plate.

The predetermined pattern of each of the membranes corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate. Each of the plurality of micro-orifices has a diameter from about 10 microns to about 50 microns. The membranes are made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone. In other embodiments of these devices, the at least one chamber comprises a plurality of chambers. The plurality of chambers may be attached to each other. The plurality of attached chambers may be arranged in a grid or may be arranged as a strip.

In all of these embodiments, the plurality of chambers define a pitch relative to one another that matches a pitch of a standard microtiter plate. The plurality of chambers define a pitch relative to one another that matches of pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate. In other embodiments, the delivery device is a microfluidic device, a pipette or a robotic device. In further embodiments, each of the plurality of micro-orifices define walls, and wherein the device further comprises a surface coating on the walls of at least one of the plurality of micro-orifices. The device may further comprise a filter layer disposed in the second region of the at least one chamber. The filter layer defines a plurality of micro-pores each having a diameter of about 2 microns to about 5 microns. The filter layer is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone. In other embodiments of the device, the device may have a trans-configuration, wherein at least one of the plurality of membranes is substantially horizontal in a test orientation of the device or the device may have a cis-configuration, wherein at least one of the plurality of membranes is substantially vertical in a test orientation of the device.

In one example embodiment, this invention provides a device comprising: a housing defining at least one chamber therein; a means for controlling fluid flow disposed in the at least one chamber and defining a plurality of micro-orifices, the means for controlling fluid flow being configured such that each of the plurality of micro-orifices is adapted to receive a single cell therein, and such that the at least one chamber includes a first region on one side of the means for controlling fluid flow, and a second region on another side of the means for controlling fluid flow; a fluid delivery means in fluid communication with the first region of the at least one chamber, the fluid delivery means being adapted to deliver a fluid to the first region; a fluid removal means in fluid communication with the second region of the at least one chamber, the fluid removal means being adapted to remove a fluid from the second region.

In further embodiments of the above-described device, the housing and the means for controlling fluid flow are configured such that fluid is adapted to pass from the first region to the second region through the plurality of micro-orifices. In other embodiments of the device, the housing and the means for controlling fluid flow are configured such that fluid is adapted to pass from the first region to the second region only through the plurality of micro-orifices. I these devices, the plurality of micro-orifices are arranged in a predetermined pattern that corresponds to a pitch of a standard microtiter plate. The predetermined pattern corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

In other embodiments of the device, each of the plurality of micro-orifices has a diameter from about 10 microns to about 50 microns. In further embodiments, the means for controlling fluid flow is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone. In additional embodiments, the at least one chamber comprises a plurality of chambers. The plurality of chambers may be attached to each other. The plurality of attached chambers may be arranged in a grid or may be arranged as a strip. In these devices, the plurality of chambers define a pitch relative to one another that matches a pitch of a standard microtiter plate. The plurality of chambers define a pitch relative to one another that matches of pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

In further embodiments of the device, the fluid delivery means is a microfluidic device, a pipette, or a robotic device. Each of the plurality of micro-orifices define walls, and wherein the device further comprises a surface coating on walls of at least one of the plurality of micro-orifices. I other embodiments of the device, the device further comprises a filter means for controlling fluid flow disposed in the second region of the at least one chamber. The filter means for controlling fluid flow defines a plurality of micro-pores each having a diameter of about 2 microns to about 5 microns. The filter means for controlling fluid flow is made of a material selected from the group consisting of glass, polymer, co-polymer, urethanes, rubber, molded plastic, polymethyl-methacrylate (PMMA), polycarbonate, polytetrafluoroethylene (TEFLON), polyvinylchloride (PVC), polymethylsiloxane (PDMS), and polysulfone. In additional embodiments of the devices provided, the device may have a trans-configuration, the means for controlling fluid flow being substantially horizontal in a test orientation of the device or the device may have a cis-configuration, the means for controlling fluid flow being substantially vertical in a test orientation of the device.

In a further example embodiment, this invention provides a microfluidic network, said network being adaptable for integration with a device for coculturing on a cell culture support surface of the device, said network comprising: a plurality of channels, the channels being adapted to deliver at least one agent to the cell culture support, and a removal device, the removal device being adapted to remove at least one analyte from the cell culture support.

In one embodiment of the above-described microfluidic network, the at least one agent is culture medium, at least one assay reagent, or a test compound. In another embodiment of the network, the at least one analyte is a waste product of cellular coculture, an assay product, or a metabolite of a test compound. The microfluidic network may be adapted to be overlaid on the cell culture support surface of the device. In an alternate embodiment, the microfluidic network is an integral part of the device for coculturing.

In yet another example embodiment, this invention provides a method of analyzing an effect of candidate compound on a cellular coculture, said method comprising: a) coculturing at least two different cell types in a two-dimensional coculture device; b) contacting at least one cell type with a therapeutically effective dose of at least one test compound for a therapeutically effective time period; c) removing at least one analyte of the coculture; and d) performing an assay on the at least one analyte.

In an embodiment of the method provided, the method further comprises microscopically analyzing the coculture for signs of cellular stress, compound toxicity, cell viability or cell death.

In other embodiments, the method further comprises histochemically staining cells of the coculture to permit visualization of intracellular structures of the cells. In further embodiments of

the method, the assay measures secretion or metabolism of a biomolecule or expression of a protein. For example, the biomolecule may be urea or ammonia. In another example, the protein may be liver albumin, beta galactosidase or a cytochrome P450 enzyme. In another embodiment of the method, the method further comprises measuring activity of a cytochrome P45 enzyme.

In another embodiment, the assay may measure expression of a nuclear receptor. The therapeutically effective dose is a low dose and the time period is from at least several weeks to several months. As used herein, therapeutically effective dose is defined as a dose which accomplishes the desired therapeutic effect in the diseased cells, tissues or organs, e.g., a therapeutically effective amount of a chemotherapeutic agent will kill cancer cells with minimal or no damage to noncancerous cells. Likewise, a therapeutically effective amount of anti-infective agent, such as an anti-viral agent will kill the targeted virus in the infected cell with little or no damage to the infected cell. The time periods of long-term test agent exposure used in the methods and assays of the present invention resemble the treatment regimen of standard treatment regimens, i.e., long-term exposure to the therapeutic agent in smallest effective doses, rather than single high doses. In another embodiment, the assay measures oxygen tension, temperature or shear flow. In an example embodiment of the method, at least one cell type is a hepatocyte and at least one second cell type is a fibroblast.

In another embodiment of the method, the method further comprises i) harvesting hepatocytes from the coculture; and ii) measuring expression of liver proteins or levels of intracellular metabolites in the harvested hepatocytes. In a further embodiment of the method, prior to coculturing the hepatocyte is transfected with a reporter gene for expression with a liver protein. The protein reported with this reporter gene may be a cytochrome P450 enzyme, an epoxide hydrolase or a conjugating enzyme. In further embodiments, he conjugating enzyme is a glutathione-5-transferase enzyme, a sulfotransferase enzyme, or an N-acetyltransferase. In other embodiments of the method provided, the cells of the first tissue type or the second tissue type are primary cells, preferably, but cells cocultured may also be cultured cells, thawed cells, wherein said cells have been isolated and frozen prior to thawing or immortalized cells. In a further embodiment, the cells of the first tissue type and cells of the second tissue type are from one subject. Preferably, the subject is a mammal and most preferably the mammal is a human.

In an example embodiment of the method, the cells of the first tissue type are hepatocytes, wherein the hepatocytes are primary cancerous cells. In another embodiment of the method, the method further comprises coculturing a plurality of cocultures of the hepatocytes, wherein each coculture is contacted with at least one different test compound, wherein each test compound is a chemotherapeutic agent. In further embodiments of the method provided, the hepatocytes are from the same human. In other embodiments, the hepatocytes are each from a different human. In one embodiment of the method, each hepatocyte coculture has at least three hepatocytes. In other embodiments, the cells of the second tissue type are fibroblasts. Preferably, in other embodiments of the method, each coculture has an optimal number of fibroblasts in heterotypic cell contact with the at least three hepatocytes to provide at least 35% heterotypic cell contact. The percent of heterotypic contact is more important than a ratio of hepatocytes to fibroblasts. In another embodiment of the method, the method further comprises coculturing the hepatocyte-fibroblast cocultures with cells of a third tissue type, wherein the third tissue type is an epithelial cell. The epithelial cells may be primary cells, cultured cell, thawed cells, previously frozen or immortalized cells in the above-described method and any of the methods provided herein. In one embodiment of the method, the method further comprises measuring the invasiveness of the cancerous hepatocytes into the epithelial cells of the coculture. I further embodiments of the method, the hepatocytes, epithelial cells and fibroblasts are cocultured in a two-dimensional coculture device having a culture pattern of round islands, wherein said islands are spaced apart relative to each other. In an alternate embodiment of the method, the hepatocytes, epithelial cells and fibroblasts are cocultured in a two-dimensional coculture device having a culture pattern of strips, wherein said strips are parallel relative to each other and are spaced apart relative to each other.

In further embodiments of the method, the method further comprises coculturing a plurality of cocultures of the hepatocytes, wherein each coculture is contacted with at least one test compound, wherein the test compound is the same for each coculture. In additional embodiments of the method, the hepatocytes in each coculture are from a different human. In another embodiment of the method, each hepatocyte coculture has at least three hepatocytes. In further embodiments of the method, the cells of the second tissue type are fibroblasts. In other embodiments of the method provided, each coculture has an optimal number of fibroblasts in heterotypic cell contact with the at least three hepatocytes to provide at least 35% heterotypic cell contact. In another embodiment of the method, the method, further comprises coculturing the hepatocyte-fibroblast cocultures with cells of a third tissue type, wherein the third tissue type is an epithelial cell. The epithelial cells may be primary cells or any non-freshly isolated cell type described above, e.g., thawed. In another embodiment of the method, the method, further comprises measuring the invasiveness of the cancerous hepatocytes into the epithelial cells of the coculture. In one embodiment of the method, the hepatocytes, epithelial cells and fibroblasts are cocultured in a two-dimensional coculture device having a culture pattern of round islands, wherein said islands are spaced apart relative to each other. In another embodiment of the method, the hepatocytes, epithelial cells and fibroblasts are cocultured in a two-dimensional coculture device having a culture pattern of strips, wherein said strips are parallel relative to each other and are spaced apart relative to each other.

In an example embodiment of the method provided herein, the cells of the first tissue type may be hepatocytes, wherein the hepatocytes are primary cirrohtic hepatocytes. In a further embodiment, the at least one test compound prevents production of fibers in the hepatocyte coculture. In other embodiments of the method, the primary cells may be hepatocytes, wherein the hepatocytes are infected with an infectious disease. In further embodiments of the method, the test compound may be an anti-viral agent, an anti-bacterial agent or an anti-parasitic agent. In other embodiments of the method, the infectious disease is a hepatitis infection. In further embodiments, the hepatitis is hepatitis A, hepatitis B or hepatitis C. in a still further embodiment of the method provided, the infectious disease is an intracellular parasitic infection.

In yet another embodiment of the method provided, the coculture device used includes:

a cell culture support surface; and a microfluidic system having a removable patterning membrane disposed on the cell culture support surface and a plurality of channels for flowing cells to surfaces exposed within the channels, wherein the channels are in conformal contact with the cell culture support surface and are parallel relative to each other and spaced apart relative to each other. In further embodiments of this method, the coculture device includes: a cell culture support; and at least one removable membrane disposed on the cell culture support, wherein the membrane forms a stencil pattern on the cell culture support. In other embodiments of the method, the effect of the test compound is absorption of the compound by the cellular coculture.

In another embodiment of the method, the effect of the test compound is metabolism of the compound by the cellular coculture. In still another embodiment the effect of the test compound is permeability of the compound into a cell membrane of a cell of the cellular coculture. In a further embodiment of the method, the effect of the test compound is toxicity of the compound on the cellular coculture.

The purpose of the various cell-patterning methods is to control of differential cell interaction and heterotypic cell-cell contact (membrane contact between different types of cells). Bhatia et al. has shown that different length scales of patterning and the degree of heterotypic contact between cells have measurable effects on hepatocyte function (See, Bhatia, S. N., et al., J. Biomed. Mat. Res. vol. 34, pp. 189-199 (1997), the entire contents of which are incorporated by reference in their entirety herein, specifically methods of patterning to obtain optimal heterotypic cell-cell contact for coculture.

Cell-cell interactions are controlled by micropatterning in co-cultures, e.g., hepatocytes and 3T3 fibroblasts, preferably primary hepatocytes. Patterning methods include microfluidics, membrane stencil patterning and electrochemistry. In microfluidic methods, cells are applied to an active surface by flowing them through the channels of a microfluidic system. Different cell types can be applied sequentially through each channel. The microfluidic system membrane can be used as cellular patterning resist, by preventing cell adhesion where the fluidic channels are in conformal contact with the growth substrate. Cells are applied to surfaces exposed within the fluidic channels, bare growth substrate regions are exposed by removing the fluidic patterning membrane. Cells are then applied to the newly exposed support surface.

In membrane stencil patterning methods, a membrane stencil, e.g., a PDMS membrane blocks cell adhesion to regions of the cell culture support surface. Cells are seeded into the open portions of the stencil and allowed to adhere to the substrate surface. The process may be repeated using nested or overlapping stencil patterns to deposit cells in bare regions or in overlapping regions.

In patterning methods using electrochemistry, electroactive cytophobic SAMs, i.e., terminated with ethylene glycol (EG), are patterned onto a cell substrate to form a negative cell patterning surface on individually addressable patterned electrodes embedded within the cell growth substrate, since EG blocks cell binding. The non-coated substrate surface is capable of supporting cell binding. A first cell type is applied to the non-SAM coated regions. The electroactive cytophobic region of the SAM is desorbed from the patterned electrode surface to reveal cell adhesive regions that reflect the pattern of the electrode. Multiple electrodes (or at least one electrode) may be activated simultaneously to activate several electrode surfaces to pattern a single cell type in several areas. A second cell type is applied to the newly activated regions of the cell growth substrate, corresponding to the pattern of the activated electrodes.

Sequential activation of patterned electrodes followed by cell deposition can be used to deposit several cell types on a surface.

Electrochemically activated discrete cell plating devices and cell patterning methods using these devices are provided by the present invention. Electrochemically activated surfaces are used for creating spatially controlled cell co-cultures. That is, activation allows for the plating of different types of cells in discrete locations, on the same surface, with very high spatial resolution and temporal control. Electrochemically activated surfaces are also used for carefully controlling the onset of migration in cell migration assays. This is accomplished by employing surfaces that are initially patterned with both cytophilic and cytophobic areas. When cells are plated (cell type 1), they will adhere to and will be confined to those cytophilic areas. Subsequently, electrochemical activation is used to turn the cytophobic areas into cytophilic areas thus enabling the plated cells to migrate onto those activated areas. Activation is also used to allow for a second cell population (cell type 2) to be plated onto the same surface as the first plated population (cell type 1) in carefully controlled locations. This is an important consideration when the extension of the heterotypical interface of cell co-cultures is of importance.

The creation of a patterned surface with both cytophilic and cytophobic areas is described below. An appropriate cytophobic surface is obtained by forming an EG SAM (ethylene glycol terminated self assembled monolayer) on a gold substrate. The EG SAM prevents cell adhesion. That SAM is damaged by applying an electrochemical potential in solution. The damage is likely due oxidation of the sulfur atom attached to the gold. The damaged SAM will lose its protein resistance and will allow for cell adhesion. That is, the surface is activated.

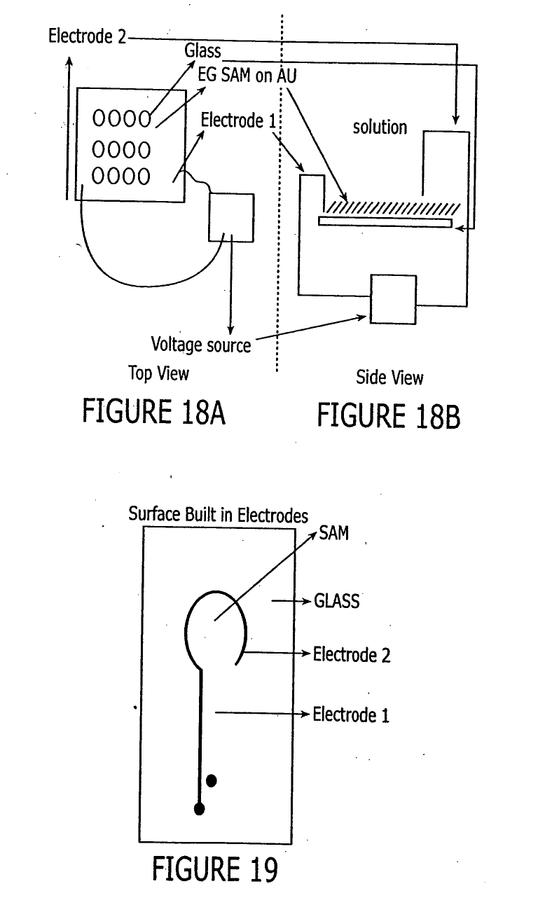

An appropriate cytophilic surface which may be used in the provided device is bare glass (SiO2), silicon, certain types of plastic such as polystyrene, or a hexadecanethiol SAM on gold (HDT), coated with fibronectin (FIGS. 18A-18B).

In order to create a surface that presents both bare glass and EG SAM areas, many techniques may be employed as described below. Photolithography may be used, followed by metallization, followed by photo resist stripping (lift off). The stripping exposes bare glass areas. After patterning, EG SAMs are formed on the gold areas.

Microcontact printing HDT SAMs on gold may also be used. The SAM acts as an etch barrier. Subsequent etching exposes bare glass. The HDT SAM is removed. An EG SAM is formed.

Photolithography on gold, followed by etching is another suitable technique to create such a surface. The photoresist acts as an etch harrier. Gold areas unprotected by the resist are etched away, exposing bare glass. Subsequently, the resist is stripped.

A physical mask may be used during metallization.

In order to create a surface that presents both HDT and EG SAM areas, many techniques may be employed, in particular, microcontact printing HDT and backfilling with EG (FIG. 21).

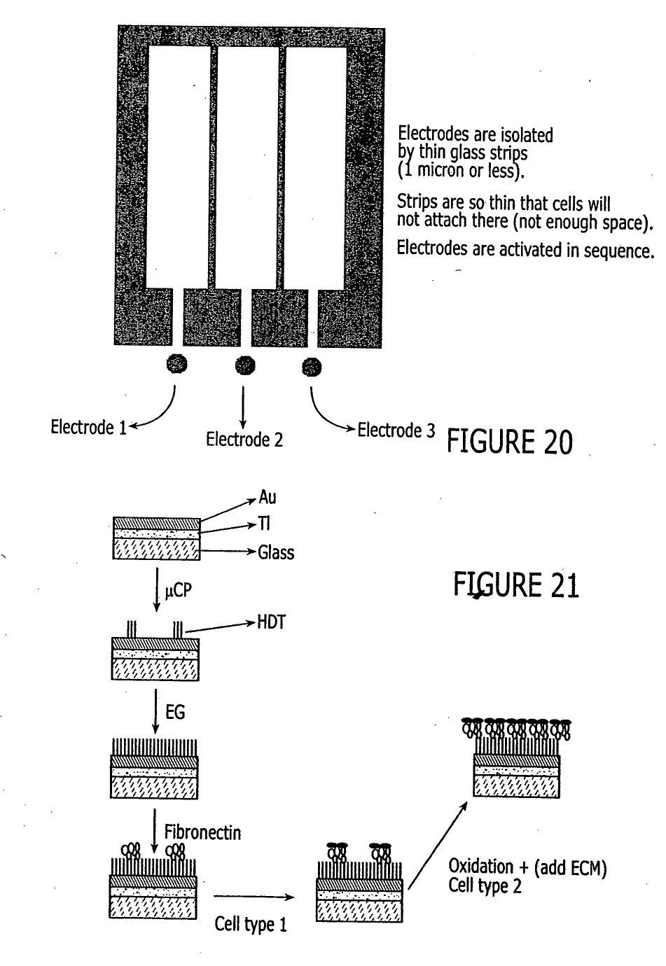

Electrochemistry based cell plating provides advantages including the following: 1) Such patterning methods yield very high spatial resolutions (sub micron). Therefore, the co-culture spatial arrangement or the spacing, shape and location of initial plating islands is very well controlled. The fabrication methods are well known and repeatable. 2) Electrochemical activation may be used for spatially controlled plating of different types of cells inside chambers or channels where stenciling is not possible. 3) The compact design requires no plating tools required (i.e., no need for stenciling membranes) (FIG. 19 and FIG. 20). 4) Cells are plated long before they are allowed to migrate or before they are exposed to a different cell population. 5) Damaging SAMs does not require careful voltage control. In general, a potential outside of ±1V vs Ag/AgCl will suffice. 6) Three or more types of cells may be plated (FIG. 20). 7) Temporal control is achieved. 8) In the case where the pattern is composed of HDT and EG SAM& the cell type 1 population is not affected by the potential because the HDT SAMs act as insulators (FIG. 21).

Other forms of electrochemical activation may be used in patterning of cocultures in the methods of the present invention. Electrochemistry is also used to cleave head groups of a SAM, exposing a previously protected ligand that acts as an adhesion promoter. The advantage of such a method is that the ligand will interact with extracellular matrix (ECM) proteins in a more specific form. One disadvantage is that the chemistry steps required to form those SAMs are more complicated.

The applications which are made possible by electrochemical based cell plating are a follows. Co-cultures are produced wherein two or more cell types are continuous and contiguous or wherein they are separated in individual islands of different cell types (FIGS. 18A-18B, FIG. 21 and FIGS. 22A-22B). Co-cultures can be produced by exposing a certain type of cell (type 1) to the patterned surface. Those cells will adhere to the cytophilic areas after a certain incubation time. Cells that were deposited on the cytophobic areas do not adhere and are removed by a rinsing step after the incubation time. After surface activation, another cell population (type 2 cells) may be introduced. These cells will preferentially adhere to the newly activated areas. Co-cultures may be used to test the effect of products secreted by one cell type on the other cell type in an environment that is physiologically relevant. Test substances may also be tested in such patterned cocultures

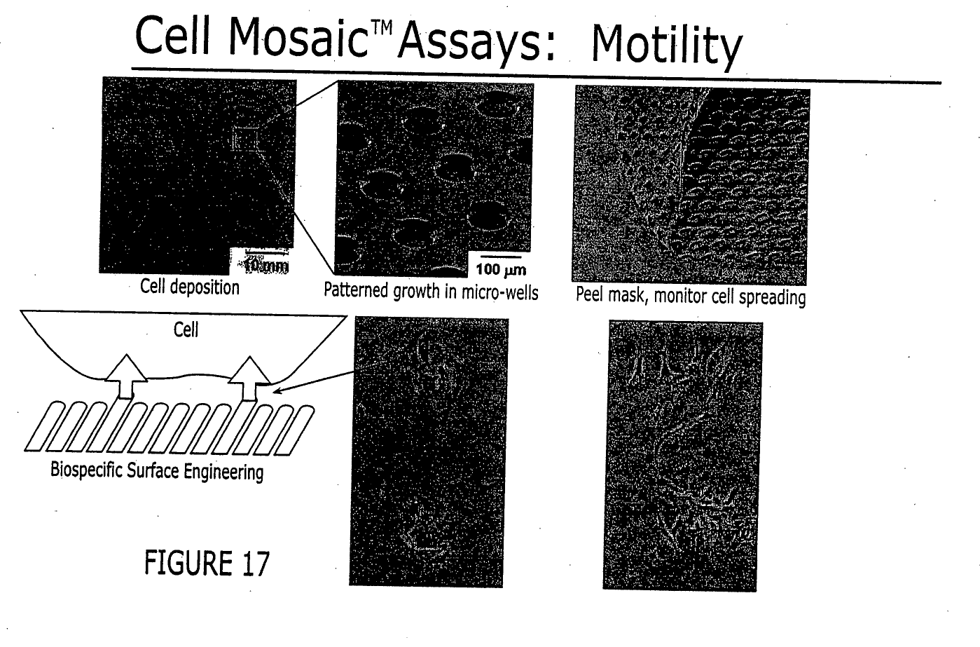

For example, cancer cells are known to secrete a growth factor called VEGF that stimulates the differentiation of endothelial cells into capillaries (angiogenesis). A co-culture (separated, or continuous, and contiguous) of cancer cells with endothelial cells may be used to test the effect of cancer cells on endothelial cells; the angiogenic response of the endothelial cells may be correlated to changes in motility as shown with cell motility assay device shown in FIG. 17. An apparatus and methods of using such apparatus in assays to monitor cell motility an cell migration have been described in copending U.S. patent application Ser. No. 10/206,111, filed Jul. 29, 2002; Ser. No. 10/206,536, filed Jul. 29, 2002; Ser. No. 10/206,112, filed Jul. 29, 2002; Ser. No. 10/206,329, filed Jul. 29, 2002 and 10/206,196, filed Jul. 29, 2002, the contents of all of which are hereby incorporated by reference in their entireties herein, specifically the cell motility and cell migration assays and apparatus for performing such assays. The assays described therein may be used, for example in the ADME/Tox devices and coculture devices provided by the present invention, and the apparatus may be adapted for use in the presently provided invention.

Treatment of the co-culture with a cancer-specific drug allows testing its effect on VEGF secretion by cancer cells with the readout of the assay being the effect on motility/angiogenesis of the endothelial cells in co-culture. Cell motility in response to chemotactic agents may also be assayed by the devices provided herein. Assays to measure or monitor chemotactic induced motility and apparatus for using such assays are described in copending U.S. Patent Applications Nos. 10/097,329, filed Mar. 15, 2002; Ser. No. 10/097,351, filed Mar. 15, 2002; Ser. No. 10/097,306, filed Mar. 15, 2002; Ser. No. 10/097,304, filed Mar. 15, 2002; Ser. No. 10/097,322, filed Mar. 15, 2002 and 10/097,302, filed Mar. 15, 2002, the contents of all of which are hereby incorporated by reference in their entireties herein, specifically the cell motility and cell migration assays used to monitor cellular response to chemotactic agents and apparatus for performing such assays.

Either or both of the cell types in the co-culture with clearly defined cell-cell boundaries may be modified with gene constructs to make it possible to perform a gene reporter assay on a pathway that is known to be modulated by the cell-cell interaction or by the interaction of one cell type with the secreted substance. Similarly, in either or both cell types, certain proteins or cellular components may be transfected with a fluorescent marker so that they may be followed during the assay.

Invasion assays, described infra, are important in studies of cancer and are carried out by the methods of the present invention by surrounding cancer cells with other relevant cell types such as endothelial cells or fibroblasts to study the ability of the cancer cells to interact with the second cell type. These studies are performed to include permanent or transient transfection of one or both cell types to increase the quality of information that is obtained from the assay, e.g., with reporter genes. In an alternative method, after activating the areas around the initial cell patches, the cancer cells are covered with a matrix that represents the type of matrix that cancer cells must burrow through during metastasis. The motility of the cells through these matrices is correlated to metastatic potential of these cells.

When using co-cultures of hepatocytes and fibroblasts to stabilize the phenotype of the hepatocytes, the fibroblasts may be transfected with a fluorescent protein that belongs to a pathway that responds to the metabolites of the drug produced by the hepatocytes. The transfection may be permanent or transient using standard methodologies. This method may be used to create co-cultures of hepatocytes and fibroblasts which have been shown to result in the maintenance of functional hepatocytes in vitro for periods of longer than two months. Hepatocyte coculture methods are described in Behnia et al. Tissue Engineering 2000, 6, 467-479; Bhatia et al. FASEB J. 1999, 13, 1883-1900; Bhatia et al. J. Biomaterials Science, Polymer Ed. 1998, 9, 1137-1160; Bhatia et al. Biotechnology Progress 1998, 14 378-387; Bhatia et al. J. Biomed. Mater. Res. 1997, 34, 189-199; and Bhatia et al. J. Cellular Engineering 1996, 1, 125-135, the contents of all of which are hereby incorporated by reference in their entireties herein, specifically the methods and materials for coculturing hepatocytes with optimal heterotypic cell contact for maintaining cell viability and functioning for time periods of at least 2 weeks up to two months, or longer. In addition to their use for clinical patient diagnostics, the electrochemically activated cell plating methods and devices of the present invention may be used by pharmaceutical and diagnostic companies for screening assays of test compounds and for rapid patient sample diagnostic assays, respectively.

Cell patterning methods which may be used in the devices and methods of the present invention are described in “Using electroactive substrates to pattern the attachment of two different cell populations” Muhammad N. Yousaf and Milan Mrksich. PNAS, May 22, 2001, vol. 98, no, 11; “Blastomeric Mask and use in Fabrication of Devices, including Pixelated-Electroluminescent Displays”, Jackman et al. U.S. patent application Ser. No. 09/694,074; Ostuni et al. Langmuir, 2000, 16, 7811 “Using membranes to pattern cells”; Ostuni et al. Langmuir 2001. In press “Deposition of cells in microwells”, the contents of all of which are hereby incorporated by reference in their entireties herein, specifically the methods and materials for cell patterning two or more cell types on electroactive surfaces.

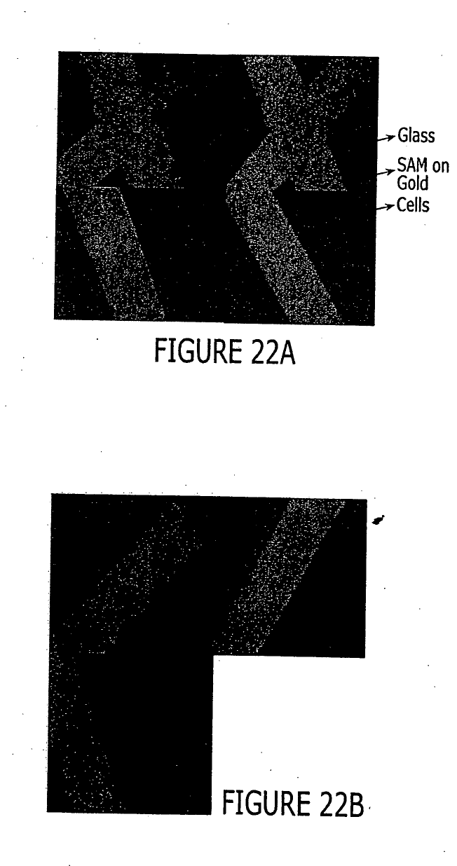

In an example of the above-described patterning coculture method and device, T47 D cells were cultured on glass in areas separated by gold-coated areas presenting an ethylene glycol terminated SAM. The cells were cultured for 24 hours (FIGS. 22A-22B, left columns) before applying a bias of 600-1300 mV. After one day in culture the cells began to migrate out of the glass surface onto the SAM surface; the images in the right columns were taken three days after applying the voltage. The migration out of the pattern is not caused by natural degradation of the SAM, because it has been shown that these cells can be maintained in a pattern separated by EG groups for more than one week.

Patterning configurations which are used in the patterning methods include round islands and elongated strips. A used herein ‘round islands’ are defined as a coculture of at least three cell of one cell type surrounded by a sufficient number of cells of at least a second cell type, wherein the cells of both cell types are contiguous and the coculture provides optimal heterotypic cell-cell contact to maintain maximum cell survival (longevity), as well as cell function, metabolism and morphology most resembling in vivo function, metabolism and morphology of said cells. Preferably, the ratio of cells is a minimum of 2:1 for fibroblast:hepatocyte culture. Other ratios may also be used. An optimal heterotypic cell-cell contact of about 35% is preferred. (See, e.g., Bhatia et al. Biotechnol. Prog. Vol. 14:378-387 (1988), the entire contents of which are incorporated by reference in their entirety herein, specifically micropatterned coculture of islands of hepatocytes and fibroblasts.)

Round islands coculture configurations may be patterned using cells from multiple sources (patients, e.g., humans) at very high density across a surface for multiplexed analysis with a single compound or assay. Spots can contain as few as three primary hepatocytes surrounded by several fibroblasts.

Patterns of coculture having elongated strips alternating lines of different cell types. As used herein ‘elongated strips’ are defined coculture of cells of at least two cell types, wherein the cocultured cells as continuous and contiguous with each other. For example, the pattern may alternate primary hepatocytes and cultured fibroblasts, but may also contain a third line of endothelial cells to more closely model the in vivo liver by promoting formation of capillary structures. Each cell line is usually 50 μm or one cell width across to maximize the amount of heterotypic contact, but the width may be altered to have multiple cells of a single type layered within a single line. Various cells can be applied to the co-culture system, but in preferred embodiments of the methods of the invention primary hepatocytes from humans are used for optimal replication of an in vivo model of the human hepatic system. Strips of endothelial cells may be added to the cocultures of primary hepatocytes and fibroblasts.

In preferred embodiments of the invention, the patterning configurations permit multiple cells from a single individual (e.g., cells from the same tissue/organ or from various organs of the same patient) to be cocultured and assayed, e.g., for long-term effects of a test compound at different doses per coculture (or different test compounds at different doses per coculture or combinations thereof) on a coculture of diseased cells. Such coculture with various test compounds will determine the optimal long-term dose of a test compound for a particular patient, to provide customized targeted therapy. In another embodiment of the present invention, cocultures of cells from multiple patients (e.g., patients having the same disease) may be cultured and assayed for long-term effects of a test compound at different doses per coculture (or different test compounds at different doses per coculture or combinations thereof). In additional embodiments of the invention, the patterning configurations include coculture of mixtures of different species (e.g., rat, mouse, porcine, and preferably human). Diseased cells (e.g., cancer, cirrhotic, infected (hepatitis) hepatocytes) may be cultured in any of the above-described patterning configurations and assayed with test compounds (e.g., chemotherapeutic agents, anti-infectives) to determine optimal long-term dosages per patient.

The co-cultured cells are viable and physiologically stable for several weeks to months under normal culture conditions. Cellular growth media may be optimized to include protease inhibitors or inhibitors of oxidative stress.

In another aspect of the present invention, provided are devices for support of cellular co-cultures. As discussed above, soft lithography allows for the fabrication of multiple-device formats for the containment of cellular co-cultures. Integrated systems for delivery of culture medium or sample compounds and removal of waste products and analytes to and from the site of cell growth may also be employed. U.S. Provisional Application No. 60/363,735, incorporated herein by reference in its entirety, described such a device with filter cups of a 96 (or more) well configuration device that holds removable macrowells for support of cells. Although this device was originally designed for use with Caco-2 endothelial cells for studying the process of compound absorption across the gastrointestinal tract, it may be used to study various other cell types. For simple metabolism assays, the device configuration is as simple as a filter membrane, or the solid base of the cell culture device. Cells are patterned on a porous filter membrane surface with PDMS microwell separations. Each macrowell of the device is removable for easy transfer of cells from one device to another without disturbing the culture surface.

In a further embodiment of the present invention additional devices for support of cellular co-cultures are provided, namely an “extravasation device” (also called a transmigration and extravasation assay device herein) support. In an embodiment example of an extravasation device, provided is a the device layered device having a lower layer containing fluid inlet and outlet receptacles connected by a microfluidic system, a top layer containing openings to the inlet and outlet receptacles as well as a cell culture well, and a porous middle membrane layer separating the cell culture well from the microfluidic network. The device may be used to culture cells on top of the porous membrane above the microfluidic system, so that the cells are exposed to material in the microfluidic system via the porous membrane. Co-cultured cells are patterned on top of the membrane system by various patterning means, e.g., any of the above-described patterning devices and methods of patterning, with sample compounds being flowed beneath the culture via the fluid inlet and outlet receptacles.

The transmigration and extravasation assay device provided by the present invention is designed to easily measure the ability of activated primary or cultured cells to extravasate through a cell monolayer in response to a chemotactic factor. Once baseline is established for the cell system, then it can be used to screen for compounds that exhibit an inhibitory effect on this biological process that occurs under conditions of inflammation, allergy, or response to infectious pathogens. Assays and apparatus using chemotaxis and therapeutic methods with chemoattractants are known to one of skill in the art and may be used in the deice provided herein. Examples of such methods and devices include the following U.S. Patents: Patent Number 1993000030764; U.S. Pat. No. 5,514,555, Assays and therapeutic methods based on lymphocyte chemoattractants; U.S. Pat. No. 4,912,057 1990-03 Guirguis et al., Cancer Diagnostics, Inc., Cell chamber for chemotaxis assay; U.S. Pat. No. 5,026,649 1991-06 Lyman et al., Costar Corporation, Apparatus for growing tissue cultures in vitro; U.S. Pat. No. 5,122,470 1992-Barnes, Floating cell culture device and method; U.S. Pat. No. 5,175,092 1992-12 Gabriels, Jr. et al., Millpore Corporation, Vitro toxicology kit and method therefore; U.S. Pat. No. 5,190,878 1993-03 Wilhelm, Apparatus for cultivating cells; U.S. Pat. No. 5,210,021 1993-05 Goodwin Jr., Neuro Probe Inc., Multiple-site chemolactic test apparatus and method; U.S. Pat. No. 5,260,210 1993-11 Rubin et al. Blood-brain barrier mode; and U.S. Pat. No. 5,302,515 1994-04 Goodwin Jr. Neuro Probe, Inc. Chemotactic test apparatus and method, the entire contents of all of which are hereby incorporated by reference in their entirety herein, specifically methods and materials used in extravasation/transmigration assays.)

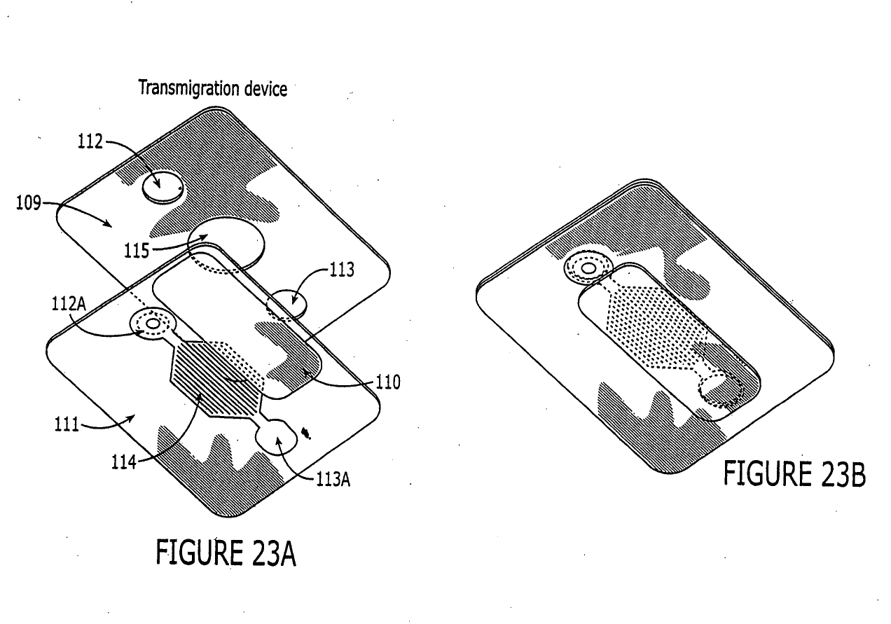

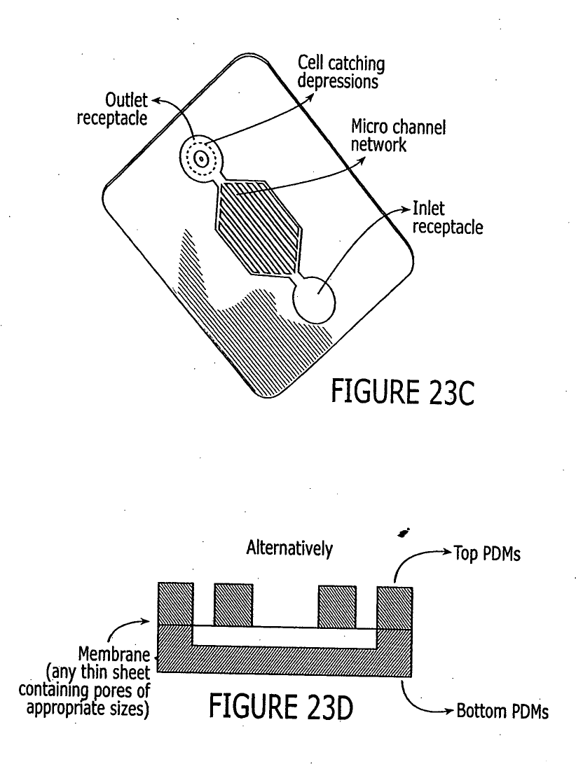

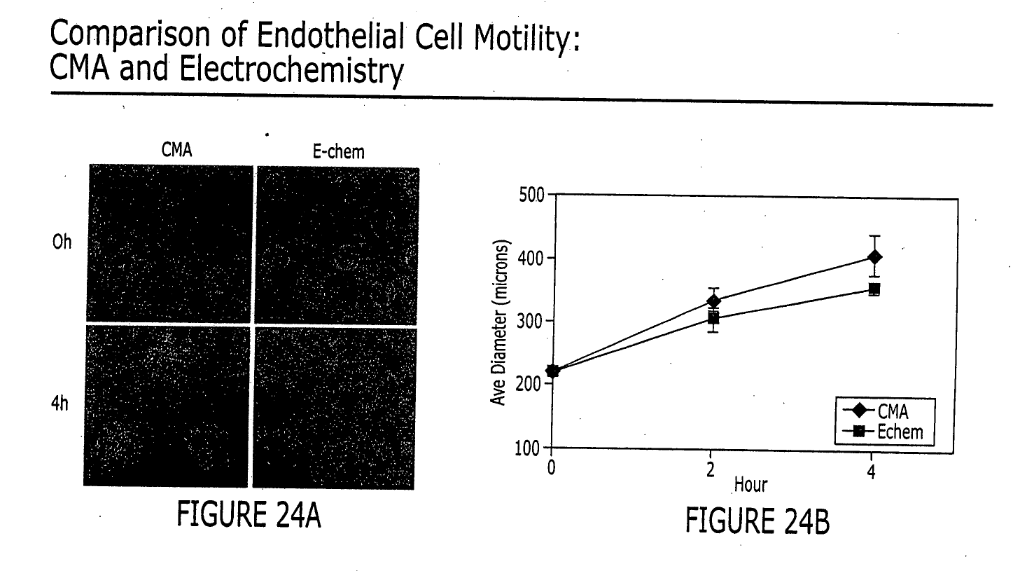

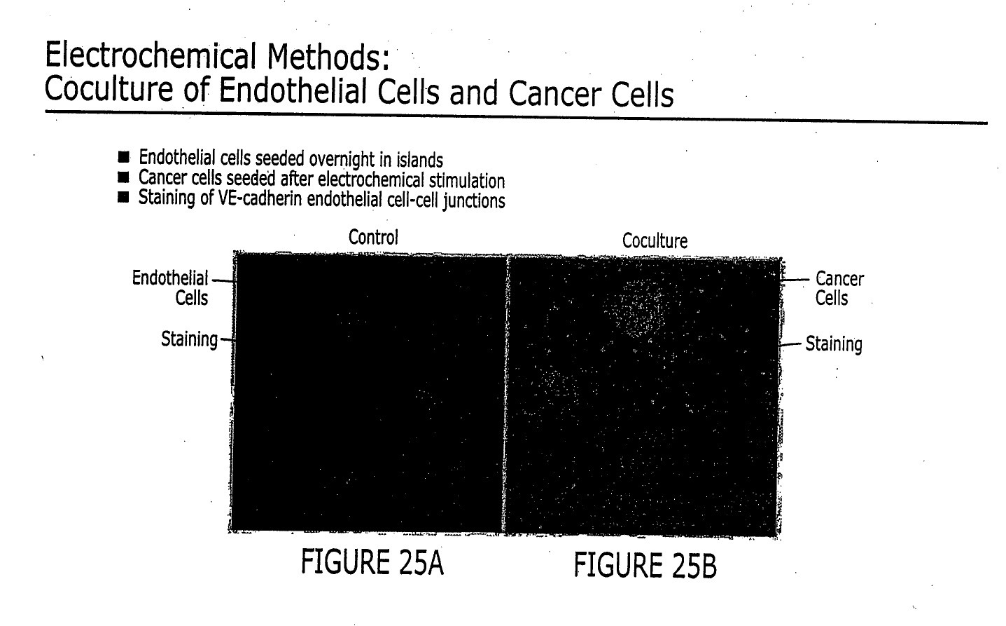

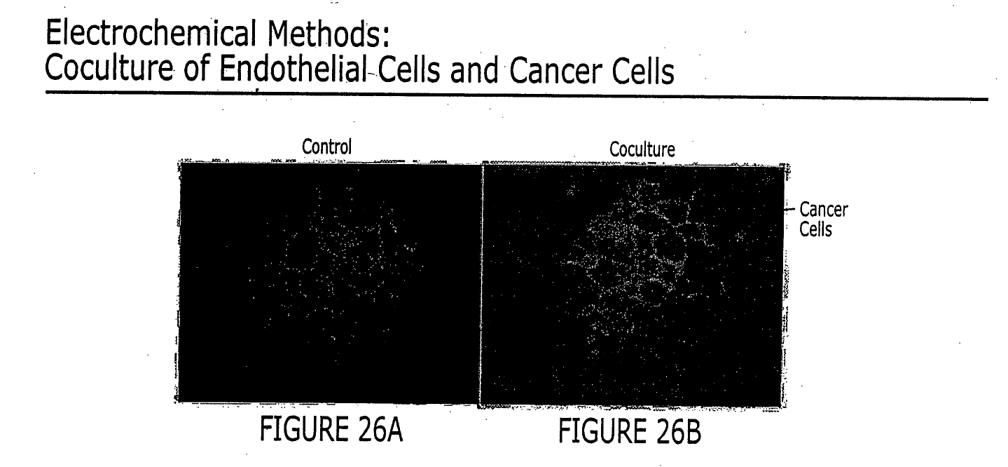

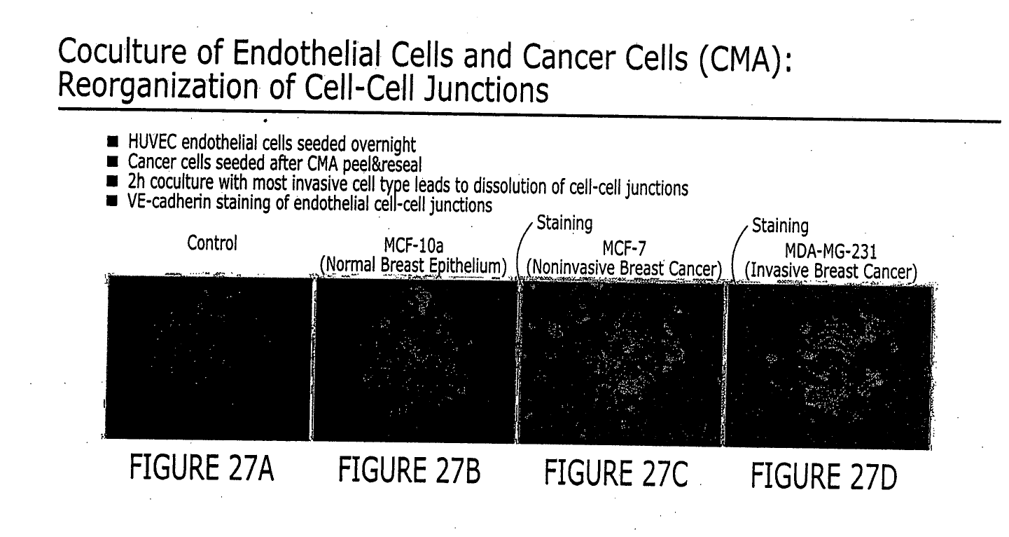

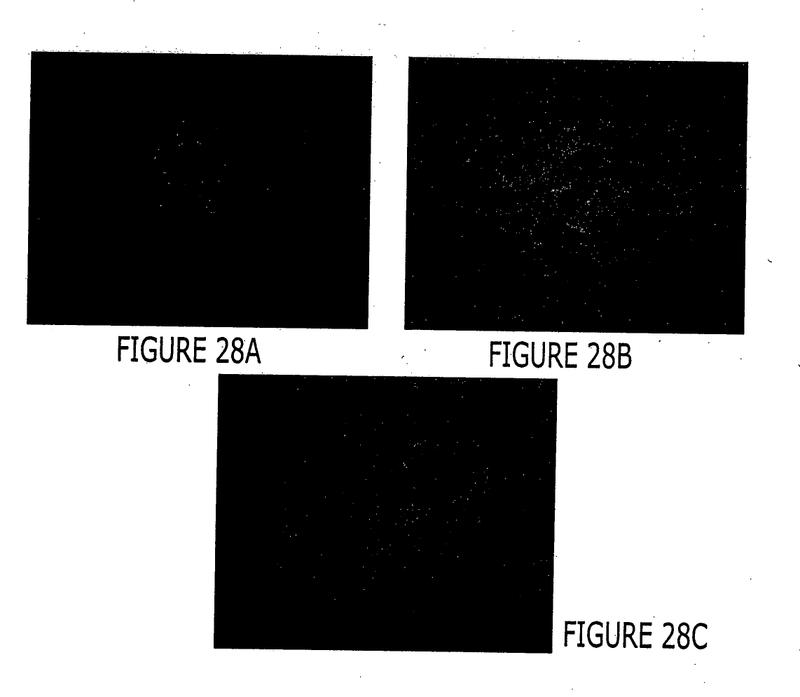

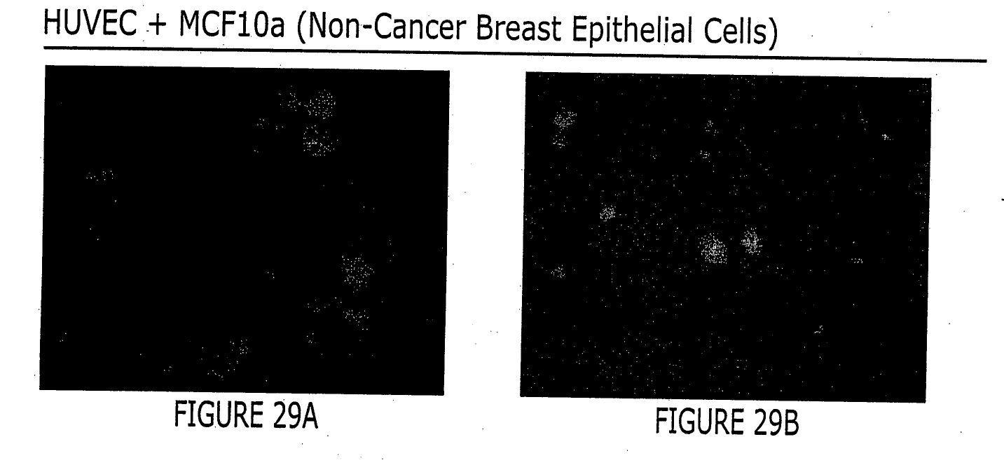

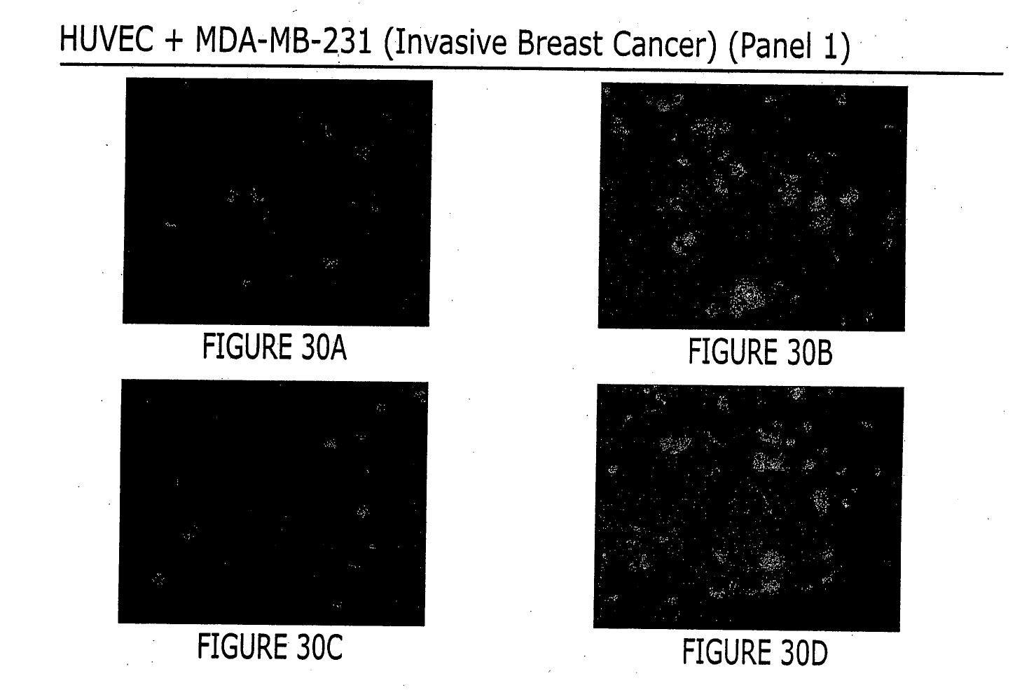

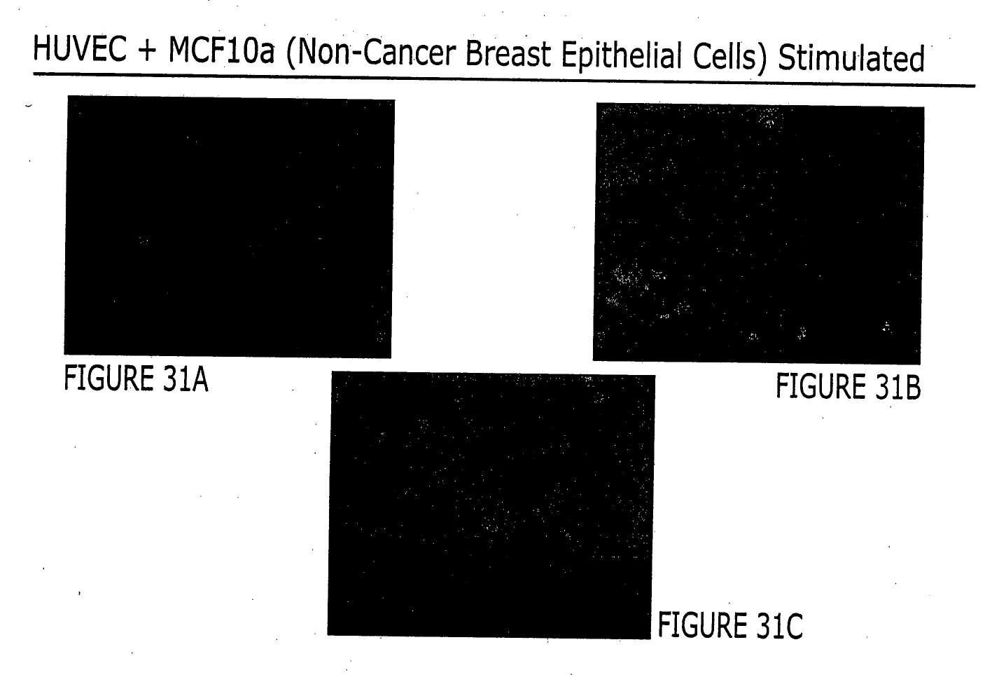

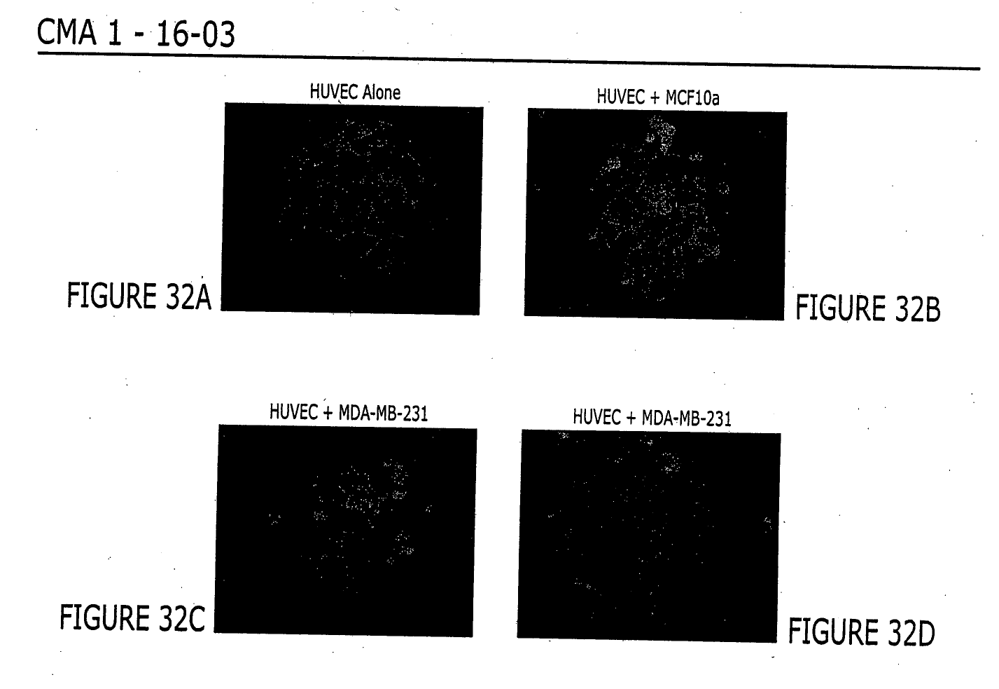

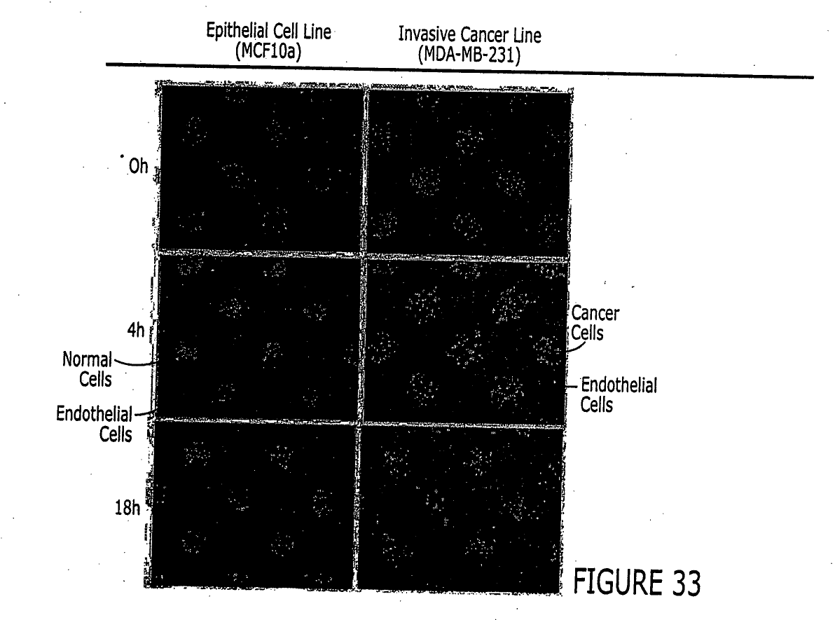

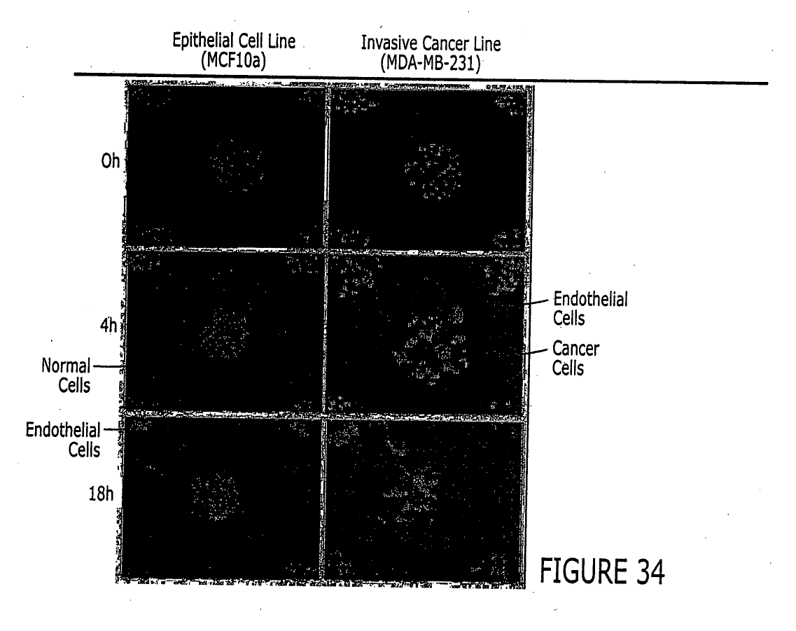

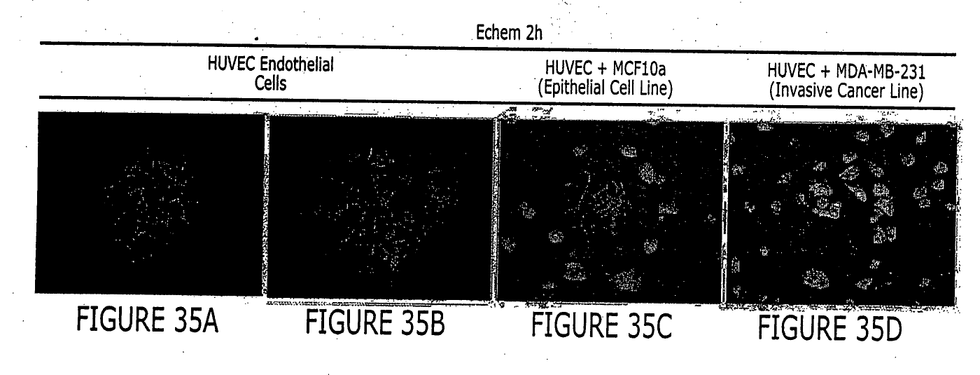

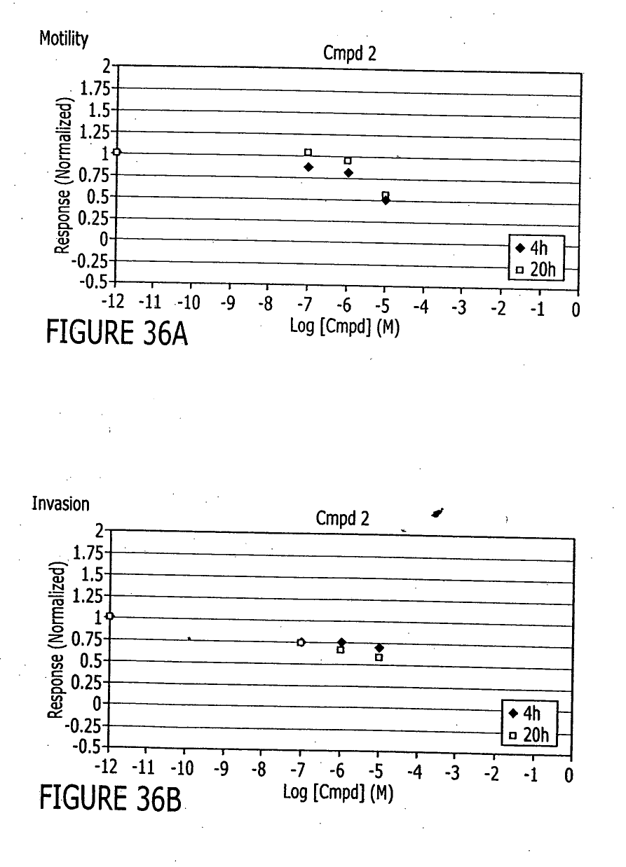

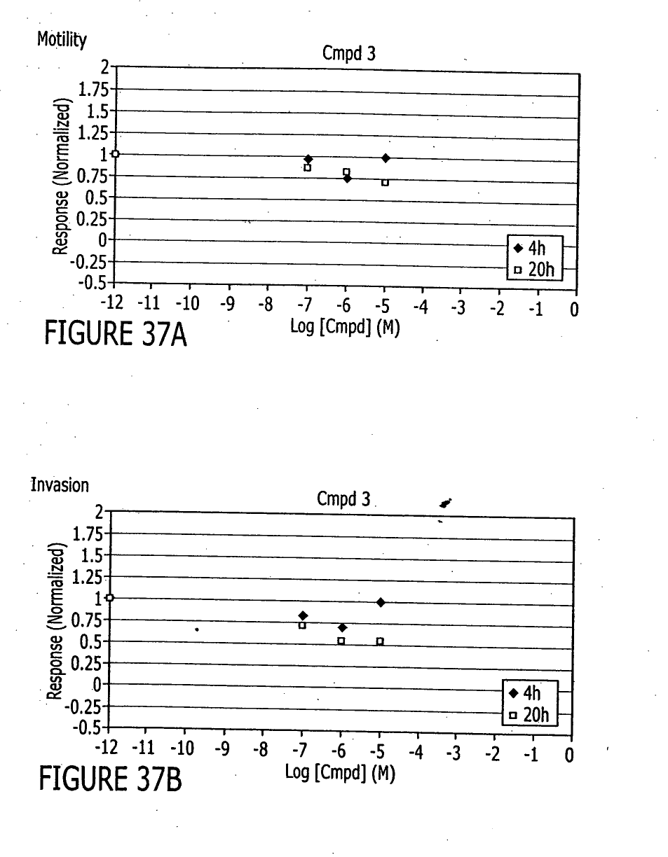

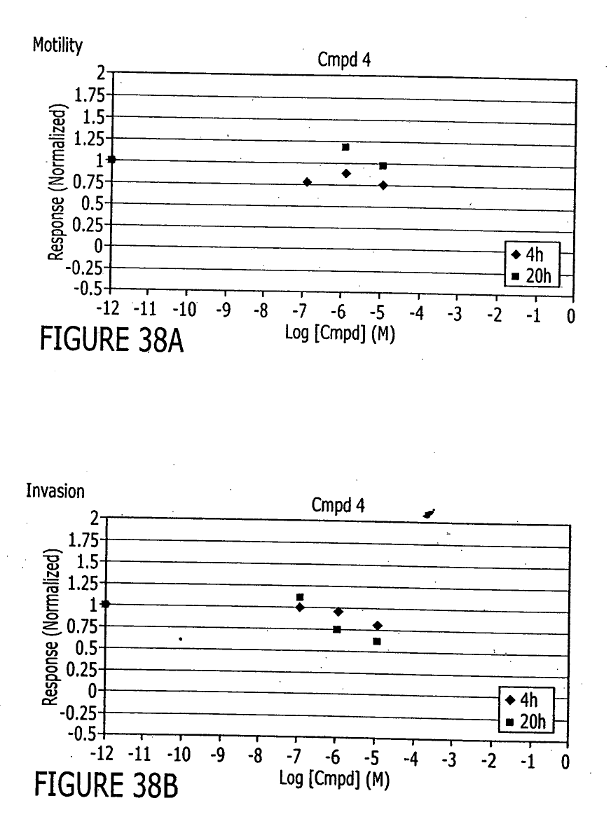

The cell extravasation assay design is described below. The assay chip is composed of three layers. The bottom layer contains two receptacles (inlet and outlet receptacles) linked by a linear and planar network of micro channels (FIG. 23C). The second or intermediate layer is composed of a membrane with small pore size (1-10 microns) that lies on top of the network. Different membranes can be used such as track-etch membranes or micro molded membranes. The top layer defines three wells in alignment with the receptacles and channel network of the bottom layer. The inlet well aligns with the inlet receptacle. The outlet well lines up with the outlet receptacle and the cell culture well is aligned with the channel network (FIG. 23A).

The extravasation membrane separates the network of channels from the cell culture well. It may also separate the inlet well from the inlet receptacle, but that is not necessary, i.e., optional. The outlet receptacle has small depressions or scaffolds on its bottom, designed to catch flowing cells that may otherwise accumulate on one extreme of the outlet receptacle after flushing. The membrane should not cover the outlet receptacle since that would hinder detection by blocking impinging light.

The membrane and channel dimensions are chosen so that the system's hydraulic resistance sets a preferential path between inlet and outlet wells during addition of the chemokine. Backflow of chemokine from the inlet well to the cell culture well is not desirable.

Track-etch membranes have been used as the porous media although micro fabricated membranes can also be used. Polyurethane or PDMS membranes with applicable pore sizes can be produced by vacuum assisted micro molding, a modality of soft lithography.

The respective layers of the device of the present invention are fabricated as follows.

The bottom layer is produced by replica molding PDMS against a micro fabricated master, typically a Silicon wafer with positive relief structures. The top layer is produced by molding.

The membrane is layered over the bottom piece in alignment with the channel network and inlet receptacle. The top layer and the bottom layer are then plasma oxidized or UV Ozone treated to prepare the mating surfaces for bonding. These surface treatments are also used for making the PDMS parts more hydrophilic, easing the task of channel filling, during assay running. After surface treatment, the top and bottom parts are brought into contact and scaled irreversibly, in alignment (FIG. 23B). An assay plate can be made with several instances of the same chip so that several experiments (e.g., 48, 96, 384) can be run in parallel. The advantages of the extravasation and transmigration device provided herein include requiring fewer cells, making it ideally suited for rare cell populations, less manipulation of the device, rapid analysis and low background.

The extravasation and transmigration devices provided herein may be used by hospitals, e.g., to assay primary or cultured cells from patients and test therapeutic compounds therein, by the pharmaceutical industry and biotech industry, e.g., to assay test compounds in coculture for optimal results and for basic research in the academic field.

In another embodiment of the present invention, microfluidic networks are integrated into the micropatterning and coculture devices for medium and sample delivery and waste and analyte removal. All of the co-culture devices provided herein may employ microfluidic networks integrated into the support structure of the device for the delivery of culture medium, assay reagents, sample compounds, etc. as well as the removal of cellular wastes, collection of assay products for analysis, etc. In alternative embodiments, microfluidic systems are provided as a separate system that is overlaid onto the co-culture surface. Clinical medicine has developed many assay systems for the analysis of drug metabolism and measurement of cellular toxicity of such compounds. The methods of cellular co-cultures for ADME/Tox analysis provided by the present invention have advantages over existing systems in that the devices/system provide miniaturization and multiplexing capabilities to allow for massive parallel processing of assay procedures on cells derived from many individuals.

In the present systems, visual methods, such as microscopy are used to assay live co-cultured cells for visual signs of cellular stress, compound toxicity, and cell death. Cellular viability may assayed by dye exclusion methods which are well known to one of skill in the art.

Apoptosis, or programmed cell death, may also be assayed by know methods. Additional methods include in vivo tagging of intracellular molecules or surface expressed molecules, such as the P-glycoprotein transporter. Further assays which may be employed include histochemical staining methods that are applied to fixed cells to highlight internal structures. Such a staining process generally kills the cells, but allows for visualization of many intracellular structures with great detail and specificity.

Metabolic assays are also used in the present methods to monitor the cellular activity of the cells in coculture. For example, hepatocyte activity and physiology may be assayed by measuring the secretion of biomolecules and proteins such as urea or liver albumin protein, as well as others. Differential conditions across the culture may be established to more closely approximate the in vivo environment of the liver. Such conditions may include, for example, gradients of oxygen tension, temperature, or shear flow. For hepatocytes, liver toxicity and death is assayed by detection of liver enzymes that are normally only found in the intracellular space.

The expression of liver proteins or the levels of intracellular metabolites is measured by harvesting hepatocyte cells from co-culture. The enzymes classically associated with drug metabolism include the oxidative enzymes of the cytochrome P450 family (especially subfamilies 1A, 2B, 2C, 2D, and 3A), and epoxide hydrolases and conjugating enzymes such as members of the glutathione-5-transferase family, sulfotransferases, N-acetyltransferase. (Caldwell, 1995) (See, Caldwell, J. et al. An introduction to drug disposition: The basic principles of absorption, distribution, metabolism, and excretion. Toxicologic Pathology vol. 23 (2), pp. 102-114 (1995), the entire contents of which are hereby incorporated by reference in their entirety herein, specifically the methods and materials for assaying various metabolic enzymes, including, but not limited to the aforementioned enzymes.) In another embodiment of the present invention, one or more cell types in the coculture may be transfected with reporter genes that are coordinately expressed with compound metabolizing enzymes to provide a measure of gene induction.

In another aspect of the present invention, provided are methods of using cellular co-culture for analysis of patient disease states. In an embodiment, primary cells are obtained from patients experiencing abnormal organ function due to disease. Other isolated cells, such as cultured cells, frozen and thawed cells, or immortalized cells may also be used for such assays. For example cultures of liver cells obtained by biopsy may be used for explanation. All of these disease systems may be incorporated into the co-culture systems and methods described above.

For example, cancer cells from a liver cancer biopsy are cultured on a coculture surface of the devices provided herein in the presence of fibroblast cells. Multiple co-cultures on a single device from a single patient are screened in a multiplexed fashion with many anti-cancer agents to determine which drug compound would be most effective at treating the patient, this “targeted therapy” is more time and cost effective than the current practice of applying broad range chemotherapies through rounds of trial and error selection before an effective treatment is found.

Invasiveness is a known hallmark of metastatic cancers. In another example embodiment of the present invention, the invasive nature of cancer cells is measured by co-culturing tumorigenic hepatocytes in the presence of a layer of endothelial cells. Similar methods may be used to determine patient specific therapeutics that minimize or eliminate invasion into epithelial layers.

Other diseases such as cirrhosis may be assayed in the coculture devices provided using the methods of the present invention. Cirrhosis is characterized by the deposition of networks of fibrous tissue that subdivide the hepatic tissue. In an embodiment of the present invention, larger co-cultures are used for the screening of compounds that prevent the production or deposition of the fibers.

Infectious diseases may also be assayed to determine optimal therapeutic test compounds in the coculture devices provided using the methods described herein. In an embodiment example of the present invention, multiplexed co-cultures can be used for high-throughput screening of therapeutics against infectious diseases, such as any of the hepatitis diseases (e.g., A, B or C), and intracellular parasites, as well as others which one of skill in the art will recognize are adaptable for coculture in the devices provided herein using the methods described infra.

In another example embodiment of the present invention, long-term exposure of cells in coculture is assayed. Long term viability and metabolic stability of cellular co-cultures allows for the measurement of long term effects of drug exposure at low dosage. This assay resembles the normal treatment regimen of a patient more closely than the current practice of measuring hepatotoxicity by exposing cells to a single high dose (acute dosing) and looking for short term damage to the tissues. Such long term low dosage studies enable the measurement of toxicity effects that arise from drug sequestration in liver cells, cumulative damage, and other problems that may only be apparent under such conditions.

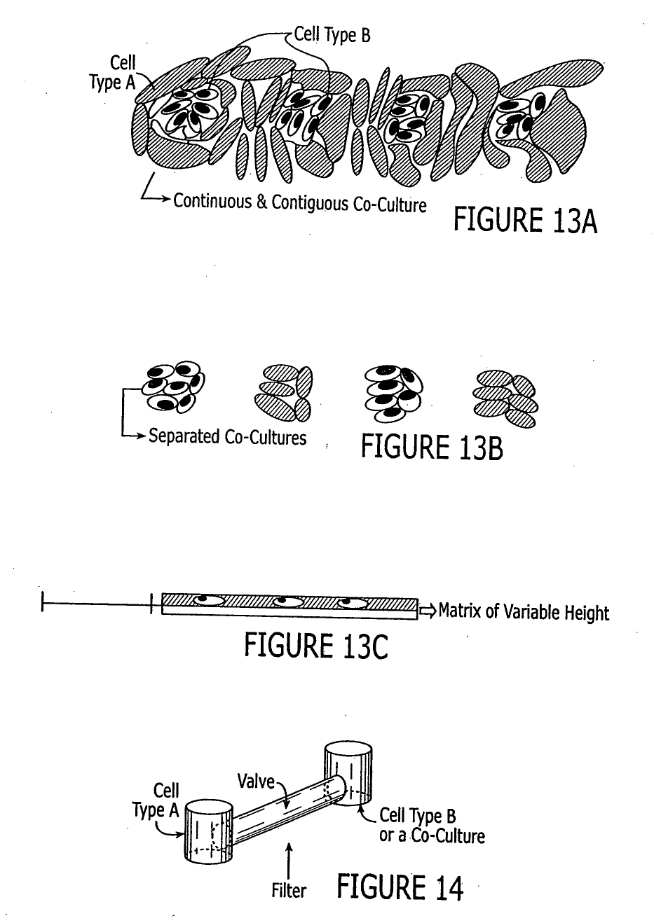

In a further embodiment of the present invention, complex cell-based assays that use co-cultures are provided. Co-cultures are produced wherein two or more cell types are continuous and contiguous or they are separated in individual islands of different cell types (FIGS. 13A-13B). Membranes may be used to pattern cells in this manner using the methods described herein. Continuous and contiguous co-cultures of two cell types are easier to produce than cultures with three different cell types; patterning three or more cell types requires three or more membranes. Cocultures may be used to test the effect of products secreted by one cell type on the other cell type in an environment that is physiologically relevant. As described above, cancer cells may be cocultured in separated, or continuous and contiguous configurations with endothelial cells to test the effect of cancer cells on the endothelial cells; the angiogenic response of the endothelial cells can be correlated to changes in motility and angiogenesis. Either or both of the cell types in the co-culture with clearly defined cell-cell boundaries may be modified with gene constructs to perform a gene reporter assay on a pathway that is known to be modulated by the cell-cell interaction or by the interaction of one cell type with the secreted substance. Similarly, in either or both cell types, certain proteins or cellular components may be transfected with a fluorescent marker so that they may be followed during the assay.

The present invention also provides co-culture based transfected arrays. Transfection of the cells to be studied in co-culture is carried out by incorporating the DNA to be transfected in the surface of the substrate. DNA of different types may be deposited in each hole of the membrane using conventional spotters so that the cells that adhere through each hole of the membrane become transfected with a different type of DNA upon exposure to the appropriate kind of transfection agent (Ziauddin et al. Nature, 2001, 411, 107-110, the entire contents of which are hereby incorporated by reference in their entirety herein, specifically methods and materials for transfection). Upon removal of the membrane, cells of a second type may be deposited on the surface to surround the cells of first type, to test the effect of the second set of cells on the first set of cells. The second set of cells may be transfected permanently or transiently.

Invasion assays for studies of cancer may be performed, as described above, by surrounding cancer cells with other relevant cell types such as endothelial cells or fibroblasts to study the ability of the cancer cells to interact with the second cell type. These studies may be performed to include permanent or transient transfection of one or both cell types to increase the quality of information that is obtained from the assay. In an alternative method, after peeling the membrane, the cancer cells may be covered with a matrix that represents the type of matrix that cancer cells must burrow through during metastasis, as described above.

When using co-cultures of hepatocytes and fibroblasts to stabilize the phenotype of the hepatocytes, the fibroblasts can be transfected with a fluorescent protein that belongs to a pathway that responds to the metabolites of the drug produced by the hepatocytes. The transfection with the fluorescentprotein may be permanent or transient using standard methodologies.

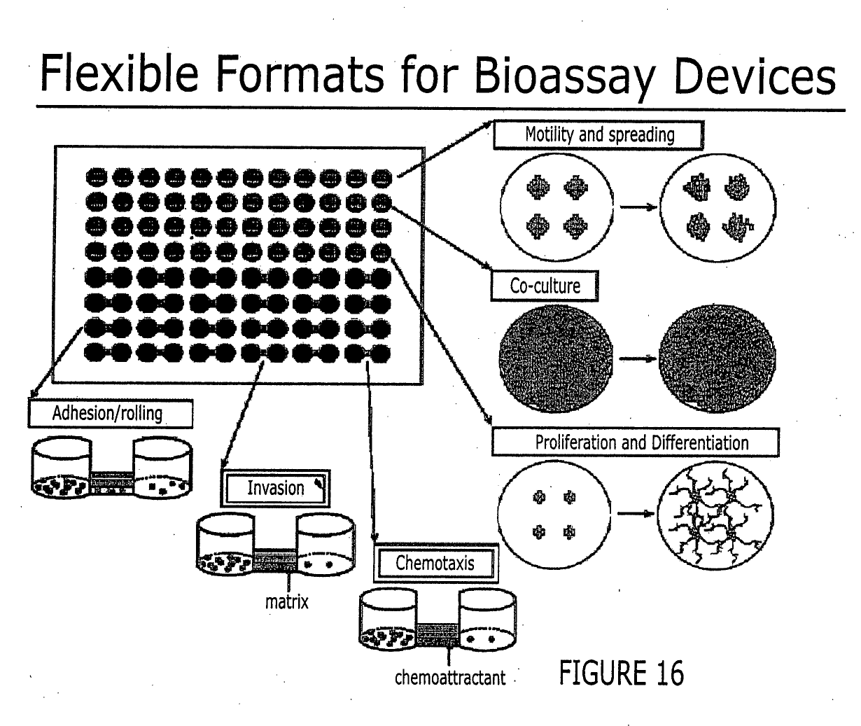

In another aspect of the methods of using coculture for ADMET/Tox analysis provided by the present invention, complex secretory pathways may be studied. Any of the devices described herein may be used, e.g., the formats illustrated in FIG. 16.

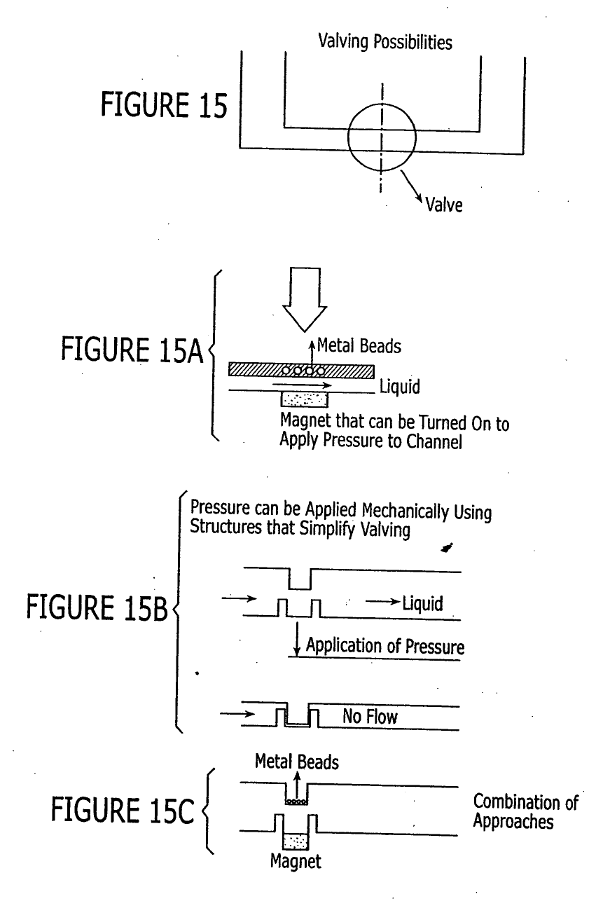

Two sample wells connected by a channel that incorporates a valve allow the study of the effect of substances that are secreted or metabolized by cells in one well on the cells in the other well. The valve in the channel that connects the two wells allows the user to define the times when the liquids in the two culture wells mix. After mixing and observing the effect of one liquid on the cells in the other well, the valve is closed and the liquids in each well are replenished or changed according to the needs of the experiment. Valving in this system may be achieved using gravitational or pressure driven flow, or by applying pressure to the channel either by mechanical means or by using magnets to pinch, i.e., close, the channel (FIG. 15). The cells in the sample wells may be in adherent layers or they may be arranged in a pattern using the devices and methods for coculture provided herein.

In an alternative use of this system, one of the sample wells may contain a co-culture of hepatocytes and fibroblasts which is known to stabilize the phenotype of the hepatocytes (see Bhatia et al. FASEB J. 1999, 13, 1883-1900; Bhatia et al. J. Biomaterials Science, Polymer Ed. 1998, 9, 1137-1160; Bhatia et al, Biotechnology Progress 1998, 14 378-387; Bhatia et al. J. Biomed. Mater. Res. 1997, 34, 189-199; and Bhatia et al. J. Cellular Engineering 1996, 1, 125-135, the entire contents of all of which are hereby incorporated by reference in their entirety herein, specifically coculture methods and materials and patterning configurations for optimal heterotypic cell contact in culture to maintain cell viability and function of several weeks or months, and more specifically such techniques are applied to hepatocyte coculture.) Additional tissue culture techniques which may be used are described by Behnia et al. Tissue Engineering 2000, 6, 467-479, which is hereby incorporated by reference in its entirety herein, specifically coculture methods for optimal cell viability. The co-culture is formed using the membrane patterning methods provided by the present invention in conjunction with a motility assay (FIG. 17). The hepatocyte co-culture that is exposed to a compound metabolizes that compound and generates by-products. The cells in the other well (i.e., a second well connected to a first well by a channel) are exposed to the metabolites of the drug from the hepatocyte co-culture to study important aspects of toxicology, metabolism, and drug-drug interaction; the valve in the channel that connects the two reservoirs (wells) may be opened at different times to test the effect of the exposure of the other cells to the metabolites of the drug. If the cells in the other (second) well are cultured in the presence of another drug that has a known effect on those cells, the system described here may be used to study the drug-drug interaction process, which is very difficult to study in vitro. There are multiple ways in which the effect of the metabolites on the pathways in the other cells may be assessed: for example, fluorescent markers, transient or permanent transfection, and changes in the rate of reaction with known substrates. The many possible configurations of an assay will be apparent to those skilled in the art of assay development. This type of device may be produced in the footprint of standard culture plates such as 24, 96, 384, 1536-well devices. The ability to generate these assays in high throughput allows the preparation of plates in which the interaction of the metabolites of a drug are tested with all the existing cell-based disease models for drug-drug interaction studies.

Microsomes and primary hepatocyte cultures offer the ability to study metabolic activity on compounds, but they suffer from having poorly defined levels of CYP enzymes that do not allow researchers to make accurate predictions of the metabolic profile of the compounds. This invention offers the ability to stabilize hepatocyte cultures while simplifying the study of the metabolites generated by the cells. In addition, the combination of the co-cultured hepatocytes inside a microfluidic system that connects them to cells of another disease model makes it possible to study drug-drug interaction processes in a manner that was not possible before.

The foregoing description and examples detail specific methods which may be employed to practice the present invention. One of skill in the art will readily know and appreciate how to devise modifications and alterations thereto and alternative reliable methods at arriving at the same information by using and/or modifying the disclosure of the present invention using ordinary skill. However, the foregoing description and examples should not be construed as limiting the overall scope of the present invention, but are to be considered as illustrative thereof. It is intended that the present invention be construed as including all such modifications and alterations insofar as they come within the scope of the appended claims or the equivalence thereof. All documents and publications cited herein are expressly incorporated by reference in their entireties into the subject application. Having set forth the example embodiments, the present invention is now claimed as follows.

1-66. (canceled)

67. A test device comprising:
a cell culture support comprising a patterning membrane disposed in the cell culture support and defining a plurality of micro-orifices, wherein each of the plurality of micro-orifices is configured to have immobilized a single cell therein, and wherein said membrane has a first side and a second side;
a delivery device in fluid communication with the first side of the patterning membrane; and
a removal device in fluid communication with the second side of the patterning membrane.

68. The device of claim 67, wherein the device is configured such that fluid is adapted to pass from the first side to the second side of the patterning membrane through the plurality of micro-orifices.

69. (canceled)

70. The device of claim 67, wherein the plurality of micro-orifices are arranged in a predetermined pattern that corresponds to a pitch of a standard microtiter plate.

71. The device of claim 70, wherein the predetermined pattern corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

72. The device of claim 67, wherein each of the plurality of micro-orifices has a diameter from about 10 microns to about 50 microns.

73. The device of claim 67, wherein the membrane is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

74. (canceled)

75. (canceled)

76. (canceled)

77. (canceled)

78. (canceled)

79. (canceled)

80. The device of claim 67, wherein the delivery device is a microfluidic device.

81. The device of claim 67, wherein the delivery device is a pipette.

82. The device of claim 67, wherein the delivery device is a robotic device.

83. The device of claim 67, wherein each of the plurality of micro-orifices define walls, and wherein the device further comprises a surface coating on the walls of at least one of the plurality of micro-orifices.

84. The device of claim 67, further comprising a filter layer disposed on the second side of the patterning membrane.

85. The device of claim 84, wherein the filter layer defines a plurality of micro-pores each having a diameter of about 2 microns to about 5 microns.

86. The device of claim 84, wherein the filter layer is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

87. The device of claim 67, wherein the device has a trans-configuration, the membrane being substantially horizontal in a test orientation of the device.

88. The device of claim 67, wherein the device has a cis-configuration, the membrane being substantially vertical in a test orientation of the device.

89. A device comprising:
a housing defining at least one chamber therein;
a plurality of membranes, each of the membranes defining a plurality of micro-orifices and being configured such that each of the plurality of micro-orifices is adapted to receive a single cell therein, the membranes being disposed in the at least one chamber such that the at least one chamber includes a first region on one side of the membranes, and a second region on another side of the membranes;
a delivery device in fluid communication with the first region of the at least one chamber, the delivery device being adapted to deliver a fluid to the first region; and a removal device in fluid communication with the second region of the at least one chamber, the removal device being adapted to remove a fluid from the second region.

90. The device of claim 89, wherein the housing and the membranes are configured such that fluid is adapted to pass from the first region to the second region through the plurality of micro-orifices.

91. (canceled)

92. The device of claim 89, wherein at least two of the plurality of membranes are substantially parallel relative to each other.

93. The device of claim 92, wherein each of the plurality of membranes are substantially parallel relative to each other.

94. The device of claim 89, wherein at least two of the plurality of membranes are spaced apart relative to each other.

95. The device of claim 94, wherein each of the plurality of membranes are spaced apart relative to each other.

96. The device of claim 89, wherein the plurality of micro-orifices of each of the membranes are arranged in a predetermined pattern that corresponds to a pitch of a standard microtiter plate.

97. The device of claim 96, wherein the predetermined pattern of each of the membranes corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

98. The device of claim 89, wherein each of the plurality of micro-orifices has a diameter from about 10 microns to about 50 microns.

99. The device of claim 89, wherein the membranes are made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

100. The device of claim 89, wherein the at least one chamber comprises a plurality of chambers.

101. The device of claim 100, wherein the plurality of chambers are attached to each other.

102. The device of claim 101, wherein the plurality of attached chambers are arranged in a grid.

103. The device of claim 101, wherein the plurality of attached chambers are arranged as a strip.

104. The device of claim 100, wherein the plurality of chambers define a pitch relative to one another that matches a pitch of a standard microtiter plate.

105. The device of claim 104, wherein the plurality of chambers define a pitch relative to one another that matches of pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

106. The device of claim 89, wherein the delivery device is a microfluidic device.

107. The device of claim 89, wherein the delivery device is a pipette.

108. The device of claim 89, wherein the delivery device is a robotic device.

109. The device of claim 89, wherein each of the plurality of micro-orifices define walls, and wherein the device further comprises a surface coating on the walls of at least one of the plurality of micro-orifices.

110. The device of claim 89, further comprising a filter layer disposed in the second region of the at least one chamber.

111. The device of claim 110, wherein the filter layer defines a plurality of micro-pores each having a diameter of about 2 microns to about 5 microns.

112. The device of claim 110, wherein the filter layer is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

113. The device of claim 89, wherein the device has a trans-configuration, wherein at least one of the plurality of membranes is substantially horizontal in a test orientation of the device.

114. The device of claim 89, wherein the device has a cis-configuration, wherein at least one of the plurality of membranes is substantially vertical in a test orientation of the device.

115. A device comprising:
a housing defining at least one chamber therein;
a means for controlling fluid flow disposed in the at least one chamber and defining a plurality of micro-orifices, the means for controlling fluid flow being configured such that each of the plurality of micro-orifices is adapted to receive a single cell therein, and such that the at least one chamber includes a first region on one side of the means for controlling fluid flow, and a second region on another side of the means for controlling fluid flow;
a fluid delivery means in fluid communication with the first region of the at least one chamber, the fluid delivery means being adapted to deliver a fluid to the first region;
a fluid removal means in fluid communication with the second region of the at least one chamber, the fluid removal means being adapted to remove a fluid from the second region.

116. The device of 115, wherein the housing and the means for controlling fluid flow are configured such that fluid is adapted to pass from the first region to the second region through the plurality of micro-orifices.

117. (canceled)

118. The device of claim 115, wherein the plurality of micro-orifices are arranged in a predetermined pattern that corresponds to a pitch of a standard microtiter plate.

119. The device of claim 118, wherein the predetermined pattern corresponds to a pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

120. The device of claim 115, wherein each of the plurality of micro-orifices has a diameter from about 10 microns to about 50 microns.

121. The device of claim 115, wherein the means for controlling fluid flow is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

122. The device of claim 115, wherein the at least one chamber compris es a plurality of chambers.

123. The device of claim 115, wherein the plurality of chambers are attached to each other.

124. The device of claim 123, wherein the plurality of attached chambers are arranged in a grid.

125. The device of claim 123, wherein the plurality of attached chambers are arranged as a strip.

126. The device of claim 122, wherein the plurality of chambers define a pitch relative to one another that matches a pitch of a standard microtiter plate.

127. The device of claim 122, wherein the plurality of chambers define a pitch relative to one another that matches of pitch of a 6-well microtiter plate, a 12-well microtiter plate, a 24-well microtiter plate, a 96-well microtiter plate, a 384-well microtiter plate, a 1,536-well microtiter plate, and a 9,600-well microtiter plate.

128. The device of claim 115, wherein the fluid delivery means is a microfluidic device.

129. The device of claim 115, wherein the fluid delivery means is a pipette.

130. The device of claim 115, wherein the fluid delivery means is a robotic device.

131. The device of claim 115, wherein each of the plurality of micro-orifices define walls, and wherein the device further comprises a surface coating on walls of at least one of the plurality of micro-orifices.

132. The device of claim 115, further comprising a filter means for controlling fluid flow disposed in the second region of the at least one chamber.

133. The device of claim 132, wherein the filter means for controlling fluid flow defines a plurality of micro-pores each having a diameter of about 2 microns to about 5 microns.

134. The device of claim 132, wherein the filter means for controlling fluid flow is made of a material selected from the group consisting of glass, polymer, co-polymer, urethanes, rubber, molded plastic, polymethylmethacrylate (PMMA), polycarbonate, polytetrafluoroethylene (TEFLON), polyvinylchloride (PVC), polymethylsiloxane (PDMS), and polysulfone.

135. The device of claim 115, wherein the device has a trans-configuration, the means for controlling fluid flow being substantially horizontal in a test orientation of the device.

136. The device of claim 115, wherein the device has a cis-configuration, the means for controlling fluid flow being substantially vertical in a test orientation of the device.

137-202. (canceled)
